(19) United States
(12) Patent Application Publication
(10) Pub. No.: US 2025/0154238 A1
(43) Pub. Date: May 15, 2025

(54) METHODS AND COMPOSITIONS FOR TREATING AND PREVENTING FIBROSIS

(71) Applicant: YALE UNIVERSITY, New Haven, CT (US)

(72) Inventors: Ian ODELL, Guilford, CT (US); Richard FLAVELL, Guilford, CT (US)

(73) Assignee: YALE UNIVERSITY, New Haven, CT (US)

(21) Appl. No.: 18/843,861

(22) PCT Filed: Mar. 3, 2023
(86) PCT No.: PCT/US23/14512

§ 371 Date: Sep. 4, 2024

Related U.S. Application Data
(60) Provisional application No. 63/316,691, filed on Mar. 4, 2022.
(60) Provisional application No. 63/316,704, filed on Mar. 4, 2022.

(51) Int. Cl. C07K 16/22; A61K 39/00; A61P 19/04

(52) U.S. Cl. CPC C07K 16/22; A61P 19/04; A61K 2039/505; C07K 2317/24; C07K 2317/569; C07K 2317/76; C07K 2317/92

(57) ABSTRACT

The present disclosure provides methods and compositions that involve inhibiting an activity of epiregulin in a cell, as well as reversing or preventing a change(s) in a cell, associated with a disease or disorder, e.g., a fibrotic disease or disorder, comprising use of an inhibitors) of epiregulin activity. Isolated antibodies, or antigen-binding fragments thereof, that specifically bind to epiregulin are provided herein as example inhibitors of epiregulin activity. Methods and compositions useful for treating or preventing a fibrotic disease or disorder in a subject, for example, comprising an inhibitor(s) of epiregulin activity, are also disclosed. The disclosure further comprises kits comprising compositions useful in practice of the disclosed methods.

### CROSS-REFERENCE TO RELATED APPLICATIONS

This application claims benefit of Provisional U.S. Application No. 63/316,704, filed Mar. 4, 2022, and Provisional U.S. Application No. 63/316,691, filed Mar. 4, 2022, the contents of each of which are incorporated by reference in their entireties for all purposes.

### STATEMENT REGARDING FEDERALLY SPONSORED RESEARCH

This invention was made with government support under AR007016 awarded by National Institutes of Health. The government has certain rights in the invention.

### FIELD OF THE INVENTION

The present invention relates to methods and compositions that involve inhibiting an activity of epiregulin in a cell, as well as reversing or preventing change(s) in a cell, due to a disease or disorder, e.g., a fibrotic disease or disorder, comprising use of an inhibitor of epiregulin activity. Methods and compositions useful for treating or preventing a fibrotic disease or disorder in a subject, for example, comprising an inhibitor of epiregulin activity, are also disclosed. The invention further provides kits comprising compositions useful in the practice of the invention.

### BACKGROUND

Pathologic fibrosis is a common final outcome of most human chronic inflammatory diseases and has been estimated to underlie almost half of all human deaths worldwide (1). Despite the critical importance of fibrosis for wound healing, there is a major unmet medical need to identify effective antifibrotic therapies; yet the inability to precisely identify the dysregulated molecular circuits that drive fibrosis impedes drug development. Systemic sclerosis (SSc/scleroderma) is the prototypic human fibrotic disease, which most commonly affects the skin, but can also affect the lungs, kidneys, gastrointestinal tract, and heart. There are no US Food and Drug Administration (FDA)-approved therapies for SSc skin fibrosis and only two approved treatments for SSc lung fibrosis (nintedanib (2) and tocilizumab (3)) that merely retard, but do not reverse disease.

Receptor tyrosine kinases (RTKs) such as platelet-derived growth factor receptor alpha (PDGFRα) (4) and fibroblast growth factor receptor 3 (FGFR3) (5) play an essential role in fibrosis because their activation in fibroblasts results in overexpression of extracellular matrix (ECM) gene products. Epidermal growth factor receptor (EGFR) is a member of the ErbB family of RTKs, is primarily expressed by epidermal keratinocytes and is aberrantly activated in solid cancers, such as lung and breast (6). In a recently identified SSc skin disease gene expression signature (the Scleroderma Skin Severity Score/4S), EGFR ligand expression correlated with skin fibrosis severity (7). However, direct EGFR inhibition has shown inconsistent results when tested in different mouse models of fibrosis. EGFR inhibition was reported to prevent skin, liver, and kidney fibrosis (8-10), but exacerbated lung fibrosis (11). These discrepant findings support the notion that hitherto unexplored signaling circuits and regulatory feedback loops, such as have been described between fibroblasts and macrophages in vitro (12), may play a central role in SSc-associated fibrosis.

Multiple studies have utilized single-cell RNA Sequencing (scRNA-Seq) to elucidate the lineages and origins of fibroblast populations in healthy and fibrotic skin (13-17). In recent years increased attention has also been centered on the observation that innate immunity plays an important role in fibrosis (18), with important signals from a unique subset of monocytes (19) and plasmacytoid dendritic cells (20). Skin biopsies from patients with scleroderma show dermal infiltration of CD14⁺ mononuclear cells, plasmacytoid dendritic cells and type 2 innate lymphoid cells (21-23), and fibroblasts clustered in close proximity to these cells are those that overexpress collagen genes in scleroderma (24, 25). These findings suggests that myeloid immune cells drive fibroblast collagen production through as yet undescribed mechanisms.

### SUMMARY OF THE INVENTION

As specified in the Background section above, there is a great need in the art for effective antifibrotic therapies for patients suffering from SSc and other fibrotic diseases and disorders. The present application addresses these and other needs.

In one aspect, the present disclosure provides an isolated antibody, or an antigen-binding fragment thereof, that may specifically bind to epiregulin.

In some embodiments, the isolated antibody or antigen-binding fragment may specifically bind to an epidermal growth factor (EGF)-like domain of epiregulin.

In some embodiments, the epiregulin may be human epiregulin.

In some embodiments, the isolated antibody or antigen-binding fragment may specifically bind to epiregulin with a K_{D} of less than about 1×10⁻⁹ M. In some embodiments, the isolated antibody or antigen-binding fragment may specifically bind to epiregulin with a K_{D} of less than about 1×10⁻¹⁰ M. In some embodiments, the isolated antibody or antigen-binding fragment may specifically bind to epiregulin with a K_{D} of about 3.8×10⁻¹¹ M. In some embodiments, the K_{D} may be determined using a bio-layer interferometry assay.

In some embodiments, the isolated antibody or antigen-binding fragment may neutralize epiregulin.

In some embodiments, the isolated antibody or antigen-binding fragment may inhibit epiregulin's interaction with an ErbB receptor.

In some embodiments, the ErbB receptor may be epidermal growth factor receptor (EGFR).

In some embodiments, the isolated antibody or antigen-binding fragment may inhibit epiregulin-induced proliferation of a fibroblast.

In some embodiments, the isolated antibody or antigen-binding fragment may inhibit epiregulin-induced proliferation of a fibroblast with an IC₅₀ of less than about 100 nM. In some embodiments, the isolated antibody or antigen-binding fragment may inhibit epiregulin-induced proliferation of a fibroblast with an IC₅₀ of less than about 10 nM. In some embodiments, the isolated antibody or antigen-binding fragment may inhibit epiregulin-induced proliferation of a fibroblast with an IC₅₀ of about 1.8 nM.

In some embodiments, the isolated antibody or antigen-binding fragment may not specifically bind to mouse epiregulin.

In some embodiments, the isolated antibody or antigen-binding fragment may not specifically bind to one or more other human EGFR ligands.

In some embodiments, the one or more other EGFR ligands may be transforming growth factor-alpha (TGFA), betacellulin (BTC), heparin-binding EGF-like growth factor (HB-EGF), epigen (EPGN), epidermal growth factor (EGF), and/or amphiregulin (AREG).

In some embodiments of any of the antibodies or antigen-binding fragments disclosed herein, the antibody or antigen-binding fragment may comprise three heavy chain complementarity determining regions (CDRs) (HCDR1, HCDR2 and HCDR3) contained within a heavy chain variable region (VH) which may comprise the amino acid sequence of SEQ ID NO: 1, or a sequence having at least 80% identity thereto; and/or three light chain CDRs (LCDR1, LCDR2 and LCDR3) contained within a light chain variable region (VL) which may comprise the amino acid sequence of SEQ ID NO: 6, or a sequence having at least 80% identity thereto.

In some embodiments, the isolated antibody or antigen-binding fragment may comprise three heavy CDRs (HCDR1, HCDR2 and HCDR3) contained within a VH which may comprise the amino acid sequence of SEQ ID NO: 1; and/or three light chain CDRs (LCDR1, LCDR2 and LCDR3) contained within a VL which may comprise the amino acid sequence of SEQ ID NO: 6.

In some embodiments, the isolated antibody or antigen-binding fragment may comprise an HCDR1 which may comprise the amino acid sequence of SEQ ID NO: 2, an HCDR2 which may comprise the amino acid sequence of SEQ ID NO: 3, and/or an HCDR3 which may comprise the amino acid sequence of SEQ ID NO: 4; and/or a LCDR1 which may comprise the amino acid sequence of SEQ ID NO: 7, a LCDR2 which may comprise the amino acid sequence of SEQ ID NO: 8, and/or a LCDR3 which may comprise the amino acid sequence of SEQ ID NO: 9.

In some embodiments, the isolated antibody or antigen-binding fragment may comprise a VH which may comprise the amino acid sequence of SEQ ID NO: 1, or a sequence having at least 80% identity thereto; and/or a VL which may comprise the amino acid sequence of SEQ ID NO: 6, or a sequence having at least 80% identity thereto.

In some embodiments, the isolated antibody or antigen-binding fragment may comprise a VH which may comprise the amino acid sequence of SEQ ID NO: 1, and/or a VL which may comprise the amino acid sequence of SEQ ID NO: 6.

In some embodiments, the antibody or antigen-binding fragment may be a human antibody, a monoclonal antibody, a humanized antibody, a single chain antibody, a Fab, a Fab′, a F(ab)₂, a Fv, or a scFv.

In some embodiments, the antibody may be a humanized antibody.

In some embodiments, the antibody or antigen-binding fragment may be of IgG1, IgG2, IgG3, or IgG4 isotype.

In another aspect, the present disclosure provides an isolated antibody or antigen-binding fragment thereof that competes for binding to epiregulin with any of the antibodies or antigen-binding fragments disclosed herein.

In another aspect, the present disclosure provides an isolated antibody or antigen-binding fragment thereof that binds to the same epitope as any of the antibodies or antigen-binding fragments disclosed herein.

In another aspect, the present disclosure provides an isolated polynucleotide encoding any of the isolated antibody or antigen-binding fragments disclosed herein.

In some embodiments, the isolated polynucleotide may comprise a VH-encoding nucleotide sequence of SEQ ID NO: 5, or a sequence having at least 80% identity thereto, and/or a VL-encoding nucleotide sequence of SEQ ID NO: 10, or a sequence having at least 80% identity thereto.

In some embodiments, the isolated polynucleotide may comprise a VH-encoding nucleotide sequence of SEQ ID NO: 5, and/or a VL-encoding nucleotide sequence of SEQ ID NO: 10.

In another aspect, the present disclosure provides a vector which may comprise any of the polynucleotides disclosed herein.

In another aspect, the present disclosure provides a host cell expressing any of the isolated antibody or antigen-binding fragment disclosed herein which may comprise any of the polynucleotides disclosed herein or any of the vectors disclosed herein.

In some embodiments, the cell may be a hybridoma.

In some embodiments, the antibody or antigen-binding fragment may be recombinantly produced.

In another aspect, the present disclosure provides a method of producing any of the isolated antibodies or antigen-binding fragments disclosed herein, the method may comprise culturing any of the host cells disclosed herein, and isolating any of the antibodies or antigen-binding fragments disclosed herein.

In another aspect, the present disclosure provides a chimeric antigen receptor (CAR) which may comprise: an extracellular domain which may comprise an antigen-binding moiety that specifically binds to epiregulin; a transmembrane domain; and a cytoplasmic domain which may comprise one or more signaling domains.

In some embodiments, the antigen-binding moiety that specifically binds to epiregulin may comprise any of the antibodies or antigen-binding fragments disclosed herein.

In another aspect, the present disclosure provides an immune cell which may comprise any of the CARs disclosed herein on its cell surface.

In some embodiments, the immune cell may be a T cell or a natural killer (NK) cell.

In another aspect, the present disclosure provides a pharmaceutical composition which may comprise any of the antibodies or antigen-binding fragments thereof disclosed herein, any of the polynucleotides disclosed herein, any of the vectors disclosed herein, or any of the CARs disclosed herein, or any of the immune cells disclosed herein, and a pharmaceutically acceptable carrier or diluent.

In some embodiments, the pharmaceutical composition may further comprise one or more additional therapeutic agents.

In some embodiments, the one or more additional therapeutic agents may be selected from mycophenolate mofetil, nintedanib, tocilizumab, pirfenidone, rituximab, corticosteroid (e.g., prednisone), methotrexate, and cyclophosphamide, or a combination thereof.

In another aspect, the present disclosure provides a kit which may comprise (i) any of the isolated antibodies or antigen-binding fragments disclosed herein, any of the polynucleotides disclosed herein, any of the vectors disclosed herein, or any of the CARs disclosed herein, or any of the immune cells disclosed herein, and/or (ii) packaging for the same.

In one aspect, the present disclosure provides a method of inhibiting an activity of epiregulin in a cell, comprising contacting the cell with an effective amount of an epiregulin inhibitor.

In some embodiments, the present disclosure provides a method of inhibiting an activity of epiregulin in a cell which may comprise contacting the cell with an effective amount of any of the antibodies or antigen-binding fragments disclosed herein, or any of the CARs disclosed herein, any of the immune cells disclosed herein, or any of the other epiregulin inhibitors disclosed herein.

In some embodiments, the activity of epiregulin may be epiregulin's interaction with an ErbB receptor.

In some embodiments, the ErbB receptor may be EGFR receptor.

In some embodiments of any the above-disclosed methods, the cell may be a fibroblast or pericyte.

In some embodiments, the epiregulin inhibitor may inhibit epiregulin-induced proliferation of the fibroblast.

In another aspect, the present disclosure provides a method of reversing or preventing one or more changes in a cell due to fibrosis, comprising contacting the cell with an effective amount of an epiregulin inhibitor.

In some embodiments, the present disclosure provides a method of reversing or preventing one or more changes in a cell due to fibrosis which may comprise contacting the cell with an effective amount of any of the antibodies or antigen-binding fragments disclosed herein, or any of the CARs disclosed herein, or any of the immune cells disclosed herein or any of the other epiregulin inhibitors disclosed herein.

In some embodiments, the one or more changes due to fibrosis may be, for example, without limitation, (1) elevated expression of epiregulin (EREG); (2) elevated expression of collagen type I alpha 1 chain (COL1A1); (3) elevated expression of collagen type IV alpha 1 chain (COL4A1); (4) elevated expression of collagen type VI alpha 1 chain (COL6A1); (5) elevated expression of tenascin-C (TNC); (6) elevated expression of fibronectin extra domain A (FN^{EDA}); (7) elevated expression of monocyte chemoattractant protein11 (MCP-1); (8) elevated expression of tissue inhibitor of metalloproteinase I (TIMP-1), or a combination thereof.

In some embodiments, the cell may be a fibroblast or pericyte.

In some embodiments, the cell may be a human cell.

In another aspect, the present disclosure provides a method of treating or preventing a fibrotic disease or disorder in a subject in need thereof, comprising administering to the subject a therapeutically effective amount of an epiregulin inhibitor.

In some embodiments, the present disclosure provides a method of treating or preventing a fibrotic disease or disorder in a subject in need thereof which may comprise administering to the subject a therapeutically effective amount of any of the antibodies or antigen-binding fragments disclosed herein, any of the polynucleotides disclosed herein, any of the vectors disclosed herein, or any of the CARs disclosed herein, or any of the immune cells disclosed herein, or any of the other epiregulin inhibitors disclosed herein.

In some embodiments, the administration of the epiregulin inhibitor (e.g., antibody or antigen-binding fragment) may result in reversal of the fibrotic disease or disorder.

In some embodiments, the epiregulin inhibitor (e.g., antibody or antigen-binding fragment) may be administered to the subject after the onset of the fibrotic disease or disorder.

In some embodiments, the epiregulin inhibitor (e.g., antibody or antigen-binding fragment) may be administered to the subject before the onset of the fibrotic disease or disorder.

In some embodiments, the fibrotic disease or disorder may be scleroderma, interstitial lung disease, gastrointestinal fibrosis, cardiac fibrosis, skin fibrosis, scleromyxedema, nephrogenic systemic fibrosis (nephrogenic fibrosing dermopathy), chronic graft-vs-host disease, sclerotic graft-vs-host disease. Bronchiolitis obliterans syndrome, keloid scar, or long COVID syndrome, or a combination thereof.

In some embodiments, the scleroderma may be systemic scleroderma.

In some embodiments, the scleroderma may be localized scleroderma (morphea).

In some embodiments, the interstitial lung disease may be idiopathic pulmonary fibrosis.

In some embodiments, the skin fibrosis is associated with systemic sclerosis or sclerotic graft-vs-host disease.

In some embodiments, any of the above-described method of treating or preventing a fibrotic disease or disorder in a subject may further comprise one or more additional therapeutic agents.

In some embodiments, the one or more additional therapeutic agents may be, for example, without limitation, mycophenolate mofetil, nintedanib, tocilizumab, pirfenidone, rituximab, corticosteroid (e.g., prednisone), methotrexate, UVA or UVB phototherapy, extracorporeal photopheresis, stem cell transplant, and cyclophosphamide, or a combination thereof.

In some embodiments of any of the above-described methods of treating or preventing a fibrotic disease or disorder in a subject, the subject may be human.

In any of the above-described embodiments or aspects, the epiregulin inhibitor may be an antibody or an antigen-binding fragment, a small molecule, a decoy receptor, a CAR modified cell, an aptamer, an alternative scaffold, or a combination thereof.

In some embodiments, the antibody or an antigen-binding fragment may be an anti-human epiregulin antibody AF1195 or anti-mouse/human epiregulin antibody Clone #189611.

In some embodiments, the decoy receptor may be a soluble ErbB receptor.

In some embodiments, the decoy receptor may be a soluble EGFR.

In some embodiments, the CAR modified cell may disrupt or kill an epiregulin-expressing dendritic cell.

In some embodiments, the CAR modified cell may be a CAR-T cell.

### BRIEF DESCRIPTION OF THE DRAWINGS

FIGS. 1 and 2 show workflow for immunization of Alloy mice, which generated positive titers to recombinant human epiregulin protein. Three of each Alloy mouse strain were immunized with recombinant epiregulin protein and 35 days later, epiregulin antibody titers were measured. Titers from the individual mixed and B6 mice strains are circled.

FIG. 3 shows hybridoma library generation and cloning from epiregulin-immunized Alloy Mix mice. Supernatant from hybridoma library shows epiregulin-binding antibodies by enzyme-linked immunosorbent assay (ELISA). One line shows results from 1/1000 dilution of immunized mouse serum and another line shows that of undiluted hybridoma library supernatant. Hybridoma cells were singly plated, which yielded 5 wells with supernatant containing epiregulin-binding antibody by ELISA, highlighted and labelled MI-M5.

FIG. 4 shows heavy and light chain sequences of humanized epiregulin neutralizing antibody 1 (hEreg NAb1). Complementarity determining regions 1-3 (CDRs1-3) are shown in bold.

FIG. 5 shows hEreg NAb1 inhibits human epiregulin-induced proliferation of human fibroblasts. Human foreskin fibroblasts (HFFs) were incubated with recombinant human epiregulin (rhEreg) in Dulbecco's Modified Eagle Medium (DMEM) containing 1% fetal bovine serum (FBS), n=3 per rhEreg concentration (left panel). HFF were incubated in media containing rhEreg 100 ng/ml along with commercially available rat anti-human/mouse epiregulin antibody (middle panel) or hEreg NAb1 (right panel), n=4 (middle panel), n=6 (right panel) per antibody concentration. Data points show mean and standard error, which were fit by interpolating from a sigmoidal standard curve. Proliferation was measured by green fluorescence protein (GFP) fluorescence at day 3 using CyQUANT Direct Cell Proliferation Assay (Invitrogen) on Synergy HTX plate reader (Biotek).

FIG. 6 shows hEreg NAb1 inhibits full-length epiregulin protein. HFF were incubated with increasing concentrations of hEreg NAb1 in the absence of exogenous epidermal growth factor receptor (EGFR) ligands.

FIG. 7 shows hEreg NAb1 has low cross-reactivity with mouse epiregulin. HFF were incubated with recombinant mouse epiregulin (rmEreg) for 3 days (left panel). HFF were incubated in media containing rmEreg 100 ng/ml along with rat anti-human/mouse epiregulin antibody (middle panel) or hEreg NAb1 (right panel), n=4 (middle panel), n=6 (right panel) per antibody concentration. Data points show mean and standard error, which were fit by interpolating from a sigmoidal standard curve.

FIG. 8 shows humanized epiregulin antibody does not neutralize other EGFR ligands. HFF were incubated with 5 mg/ml hEreg NAb1 alone and recombinant EGFR ligands at 0.1 ng/ml. 1 ng/ml, and 10 ng/ml for 3 days prior to quantification.

FIGS. 9A-9I show EGFR activation marks pathogenic fibroblasts in SSc skin and lung. (FIGS. 9A-9B) Uniform Manifold Approximation and Projection (UMAP) embedding of scRNA-seq data from five patients with diffuse cutaneous SSc and five healthy controls. (FIG. 9C) Heatmap of significantly upregulated collagen gene expression in SSc fibroblasts (Fib) and pericytes (PC). (FIG. 9D) Gene ontology processes identified by upregulated SSc genes in the fibroblast (Fib) and pericyte (PC) clusters. (FIG. 9E) Expression of EGFR in UMAP embedded data with clustering as in (FIG. 9A). (FIG. 9F) Heatmap of significantly upregulated genes in SSc EGFR-expressing fibroblasts compared to SSc EGFR-negative fibroblasts and healthy control fibroblasts. (FIG. 9G) t-distributed stochastic neighbor embedding (t-SNE) plot of SSc versus healthy fibroblast gene expression. (FIG. 9H) SSc and healthy skin and lung were stained with an antibody against phosphorylated EGFR at Y1068. Images are 40× magnification with 40 μm scale bar. (FIG. 9I) Enumeration of pEGFR+ cells in each condition (n=3 slides each, 10 high power fields (hpf) per slide). Heatmaps in (FIG. 9C and FIG. 9F) are log₂ (fold change) with *=P<0.05, **=P<0.01, ***=P<0.001, adjusted using the Benjamin-Hochberg correction for multiple tests. (FIG. 11) Data are means±SD (ns, not significant. * P <0.05, **** P<0.0001) analyzed with unpaired two-tailed Student's t-test.

FIGS. 10A-10G show epiregulin⁺ dendritic cells accumulate in human skin and lung fibrosis. (FIG. 10A) Sankey diagram of enriched receptor-ligand pairs in SSc skin and at least two lung scRNA-Seq datasets. Ribbon width is proportional to 1/rank of the skin SSc data. (FIG. 10B) Plot of the CellphoneDB ranks (adjusted p-values) of the interaction of EREG. AREG, and HBEGF with EGFR in skin scRNA-Seq data (SSc skin 2 (15)), keloid skin (14), and pulmonary fibrosis studies (SSc lung 1 (41), 2 (42), 3 (33)). Data are shown in the order of HBEGF, AREG, and EREG for each sample. Dotted line shows rank=0.05. (FIG. 10C) Expression of epiregulin in UMAP embedded data with same clusters as in FIG. 9A. (FIG. 10D) Heatmap of dendritic cell marker expression by SSc vs healthy epiregulin-expressing myeloid APC (pos) compared to epiregulin-cells (neg). (FIG. 10E) Photomicrographs of skin dermis and lung from SSc and healthy subject samples stained with an antibody against epiregulin. Arrowheads indicate positive cells. (FIG. 10F) Enumeration of epiregulin⁺ cells in SSc skin dermis and lung (n=3 slides each, 10 high power fields (hpf) per slide). (FIG. 10G) Immunofluorescence of SSc and healthy skin and lung stained against phospho-EGFR (pEGFR) and epiregulin (Ereg). Dashed lines delineate region of fibrotic dermis. Images are 40× magnification with 40 μm scale bar. Data are means±SD (*** P<0.001, **** P<0.0001) analyzed with unpaired two-tailed Student's t-test.

FIGS. 11A-11G show epiregulin has defined expression patterns during mouse skin and lung fibrosis. (FIGS. 11A-11C) B6 mice were injected subcutaneously with 0.2 mg bleomycin (BLM) and skin was stained for hematoxylin and eosin (FIG. 11A) and trichrome (FIG. 11B) 3 weeks later. Epidermis (epi), dermis (dermis) and dermal white adipose tissue (DWAT) are highlighted on histology. (FIG. 11C) Immunofluorescence images from the same skin with CD34 and CD45 antibodies. (FIG. 11D) Hydroxyproline content of the skin at different time points after subcutaneous bleomycin injection (n=3 per group). (FIG. 11E) Heatmap of mean of log₂ (expression fold change) of ECM genes and EGFR ligands at different time points after subcutaneous bleomycin injection compared to PBS controls, n=3 per time point. (FIG. 11F) Bulk RNA sequencing of dendritic cells isolated from fibrotic skin of Mgl2^{DTReGFPpANeo} mice 3 weeks after subcutaneous bleomycin injection compared to PBS controls (n=3 per group). (FIG. 11G) Relative expression of epiregulin at different time points after intratracheal bleomycin administration to B6 mice. Data are means±SD (*P<0.05, ** P<0.01) analyzed with one-way analysis of variance (ANOVA) with Tukey multiple-comparisons test (FIG. 11D. FIG. 11E, and FIG. 11G).

FIGS. 12A-12K show epiregulin inhibition alleviates mouse and human skin fibrosis. (FIGS. 12A-12C) As diagramed in (FIG. 12A), cohorts of B6 and Ereg^{-/-} mice were injected with bleomycin subcutaneously and 35 days later analyzed for skin thickness (FIG. 12B), with representative histology shown in (FIG. 12C). (FIGS. 12D-12K) As diagramed in (FIG. 12D), 21 days after bleomycin injection mice began treatment with epiregulin antibody compared to controls treated with PBS (NT) for two weeks. After euthanasia, skin was analyzed for dermal thickness (FIG. 12E), hydroxyproline (FIG. 12F), gene expression (FIGS. 12G-12H) and histology (FIG. 12I), with hematoxylin and eosin staining shown on the top row, trichrome staining in the middle row, and pEGFR immunohistochemistry (IHC) on the bottom row, n=8 (PBS), 11 (BLM), and 12 (Ereg Ab). (FIG. 12J) Adjacent punch biopsies from the forearm of a patient with diffuse cutaneous SSc were cultured for 9 days in media alone (NT) or with addition of epiregulin neutralizing antibody (Ereg Ab). Inset shows higher magnification of dermal collagen. (FIG. 12K) Skin explant media was analyzed for pro-COL1A1 secretion by enzyme-linked immunoassay (ELISA). Histology and immunohistochemistry (IHC) images are 20× and 40× magnification, respectively. Data are means±SD (ns, not significant. * P<0.05, ** P<0.01, *** P<0.001, **** P<0.0001) analyzed with one-way analysis of variance (ANOVA) with Tukey multiple-comparisons test (FIG. 12B and FIGS. 12E-12H) and paired two-tailed Student's t-test (FIG. 12K).

FIGS. 13A-13I show epiregulin inhibition reduces mouse and human lung fibrosis. (FIGS. 13A-13E) As diagramed in (FIG. 13A), 10 days after intratracheal bleomycin mice began treatment with epiregulin antibody for two weeks prior to euthanasia. Lungs were analyzed for histology (FIG. 13B), modified Ashcroft score (FIG. 13C), hydroxyproline (FIG. 13D) and epiregulin gene expression (FIG. 13E), n=6 (PBS), 11 (BLM), and 7 (Ereg Ab). Histology images in (FIG. 13B) are 10× magnification with 200 μm scale bars and IHC images are 40× magnification with 40 μm scale bars. (FIGS. 13F-13I) Fresh explanted lung tissue from a deceased patient donor with familial idiopathic pulmonary fibrosis was processed for histologic staining, which showed fibroblastic foci formation and hyperplasia of alveolar type II epithelial cells, indicated by arrows (top panel hematoxylin and eosin (H&E), bottom panel trichrome with 50 μm scale bar). The same tissue was cut into cubes and cultured for 10 days in the presence of the multikinase inhibitor nintedanib 0.1 μM (Nin), the Alk5 inhibitor A-1544033 (IN-1130) 10 μM (Alk5i), epiregulin antibody 2.5 μg/ml (Ereg Ab) or non-treated vehicle control (NT). (FIGS. 13G-13I) Relative expression of indicated genes, n=4 per group. (FIG. 13I) Protein secretion of indicated genes measured by ELISA, n=8 per group. ELISA samples with poor signal and qPCR outliers identified by Grubbs's test with alpha-0.05 were excluded. Data are means±SD (ns, not significant. * P<0.05, ** P<0.01, **** P<0.0001) analyzed with one-way analysis of variance (ANOVA) with Tukey multiple-comparisons test (FIGS. 13C-13E) and Dunnett's multiple-comparisons test (FIGS. 13G-13I).

FIGS. 14A-14I show type I interferon induces EGFR-NOTCH circuit between EREG⁺ DC and fibroblasts. (FIG. 14A) Expression fold change of epiregulin when THP-1 monocytes were incubated with each indicated cytokine. (FIGS. 14B-14D) Epiregulin expression fold change from freshly isolated peripheral blood CD14⁺ monocytes (FIG. 14B) and CD1c⁺ dendritic cell precursors (FIG. 14C) or cultured human bone marrow-derived dendritic cells (BMDCs) (FIG. 14D) after incubation with IFNα2. (FIG. 14E) Expression fold change of NOTCH ligands, receptors, and target genes by human foreskin fibroblasts (HFFs) incubated with recombinant epiregulin (rEreg, n=5). (FIG. 14F) Epiregulin relative expression by BMDC primed with IFNα2 prior to exposure to NOTCH ligand DLL4 (n=3-4 per time point in each group). Statistics compare each group±DLL4. (FIG. 14G) Relative expression of EGFR ligands by HFF (n=3). Genes with fewer than three points were below detectable level. (FIG. 14H) Changes in extracellular matrix (ECM) gene expression when HFF were incubated with media alone (NT) or epiregulin neutralizing antibody (Ereg Ab). FN^{EDA} refers to the extra domain A-containing isoform of fibronectin (n=5). (FIG. 14I) Model of epiregulin-NOTCH circuit between monocyte-derived DC3 and fibroblasts. Data are means±SD (ns, not significant. * P<0.05, ** P<0.01, *** P<0.001, **** P<0.0001) analyzed with unpaired two-tailed Student's t-test (FIGS. 14A-14F and FIG. 14H) and one-way analysis of variance (ANOVA) with Tukey multiple-comparisons test (FIG. 14G).

FIGS. 15A-15F show inhibition of type I interferon-EGFR-NOTCH axis prevents fibrosis in vivo. As diagramed in (FIG. 15A), B6 mice were injected with bleomycin subcutaneously, then at 2 weeks injected intraperitoneally with Ifnar1-blocking antibody (Ifnar1 Ab), isotype control antibody (iso) or PBS (NT). At 3 weeks, skin was analyzed for histology (FIG. 15B), dermal skin thickness (FIG. 15C), hydroxyproline (FIG. 15D), and gene expression (FIGS. 15E-15F). Data are aggregated from two separate experiments, with no significant differences found between NT and iso control groups, so they were combined for purpose of clarity. Data are means±SD (ns, not significant. * P<0.05, ** P<0.01, *** P<0.001, **** P<0.0001) analyzed with one-way analysis of variance (ANOVA) with Tukey multiple-comparisons test (FIGS. 15C-15F).

FIGS. 16A-16D show cell cluster signature genes and immunostains of SSc skin. (FIGS. 16A-16B) Major cell clusters and signature genes. Heatmaps of log (fold change) of gene expression of top 10 upregulated genes per cluster in aggregated scRNA-Seq data of healthy controls and SSc samples. (FIG. 16C) Healthy and SSc skin and lung were stained with Rabbit IgG isotype control antibody to compare the signal shown in FIG. 9H for anti-human pEGFR. (FIG. 16D) Immunostaining of SSc and healthy skin with pEGFR and the mesenchymal marker vimentin (20× magnification. 100 μm scale bar).

FIGS. 17A-17C show healthy and SSc skin and lung low power images. Healthy and SSc skin and lung were stained for histology by hematoxylin and eosin (H&E) (FIG. 17A), pEGFR Tyr-1068 (FIG. 17B), and epiregulin (FIG. 17C). Dashed boxes label the location of the high-power images of the skin shown in FIG. 9 and FIG. 10. Slides were imaged with a Keyence BZ-X800 microscope at 10× magnification and stitched together using their software. Scale bars are 500 μm.

FIGS. 18A-18D show EREG DC marker expression and immunophenotyping. (FIG. 18A) Diagram of enriched receptor-ligand growth factor pairs in SSc skin. (FIG. 18B) Relative expression of epidermal growth factor (EGF), factor-alpha (TGFA), betacellulin (BTC), EREG, amphiregulin (AREG), heparin binding epidermal growth factor (EGF) like growth factor (HBEGF), and epigen (EPGN) from whole tissue of healthy and SSc frozen skin (n=3 per group). (FIG. 18C) Peripheral blood mononuclear cells (PBMCs) isolated from healthy volunteers were stained for epiregulin along with adaptive and myeloid cell markers and analyzed by FACS. Gating shows live CD45⁺ cells, followed by epiregulin⁺ lin⁻ (CD3/CD20/CD66b/NKp46) cells to exclude T cells. B cells, granulocytes, and NK cells. Data is representative from 2 healthy volunteers. (FIG. 18D) Quantification of DC subsets among epiregulin⁺ cells with cDC2 including all CD1c⁺ cells and cDC1 by CD141⁺ staining. Data are means 1 SD (ns, not significant. * P<0.05, **** P<0.0001) analyzed with unpaired two-tailed Student's t-test (FIG. 18B) and one-way analysis of variance (ANOVA) with Tukey multiple-comparisons test (FIG. 18D).

FIGS. 19A-19C show Ereg^{-/-} mice develop skin fibrosis similar to wild type. Diagramed in (FIG. 19A), cohorts of B6 and Ereg^{-/-} mice were injected with bleomycin subcutaneously and 21 days later analyzed for hydroxyproline (FIG. 19B) and histology (FIG. 19C).

FIG. 20 shows NOTCH ligand nephroblastoma overexpressed (NOV) induces epiregulin expression in BMDC. Epiregulin relative expression by BMDC treated with IFNα2 prior to exposure to NOTCH ligand NOV (n=3-4 per time point in each group).

FIGS. 21A-21C show that hEreg NAb1 reduces fibrosis markers in sclerotic GvHD skin explants. Paired skin biopsies from right arm and left abdomen of two patients with sGvHD were incubated with 2.5 mg/ml hEreg NAb1 or isotype control IgG1 antibody for 10 days. (FIG. 21A) Photos of patient fibrotic skin. (FIGS. 21B-21C) Media was changed every 48 hours and the level of fibrosis protein markers in the supernatant was quantified by ELISA (Abcam kits ab213831, ab210966, ab219046, ab187394, ab179886).

FIGS. 22A-22B show the analysis of EREG expression in SSc compared to healthy controls and compared to modified Rodnan Skin Score (mRSS). The results show and increased number of EREG+ cells in SSc skin compared to healthy controls (FIG. 22A) and that EREG expression showed a significant positive correlation with disease severity by modified Rodnan Skin Score (mRSS) (FIG. 22B).

### DETAILED DESCRIPTION

Systemic sclerosis (SSc/scleroderma) is an autoimmune disease that causes skin and internal organ fibrosis. The molecular signals that drive persistent fibrosis are poorly understood. The present disclosure was driven by the hypothesis that an immune-mesenchymal signaling circuit underlies SSc-related skin and lung fibrosis, and that targeting a specific ligand may prevent RTK activation of pathologic fibroblasts, yielding a more efficacious and better-tolerated therapeutic approach than the current limited options. Through single-cell RNA sequencing (ssRNA-Seq) analysis of skin and lung tissues from patients with diffuse cutaneous SSc, epidermal growth factor receptor (EGFR) activation was identified as a marker of pathogenic fibroblasts in both organs. Examination of ligand-receptor enrichment identified dendritic cell-derived epiregulin as a central driver of fibroblast EGFR activation. In mouse models and patient explants, epiregulin was essential for the persistence of skin and lung fibrosis, which could be abrogated by epiregulin genetic deficiency or a neutralizing antibody. Mechanistically, epiregulin expression marks an inducible state of DC3 mediated by type I interferon, which drives a multicellular circuit in which EGFR induces activation of NOTCH signaling and excess extracellular matrix production. The scientific findings disclosed herein reveal epiregulin as a crucial signal that maintains skin and lung fibrosis in SSc and other fibrotic diseases.

Accordingly, the disclosure in part provides methods and compositions that involve inhibiting an activity of epiregulin in a cell, as well as reversing or preventing a change(s) in a cell associated with a disease or disorder, e.g., a fibrotic disease or disorder, comprising use of an inhibitor(s) of epiregulin activity inhibit an activity of epiregulin in a cell, as well as reversing or preventing a change(s) in a cell associated with a disease or disorder, e.g., a fibrotic disease or disorder, for example, comprising an inhibitor(s) of epiregulin activity, are also disclosed. Methods and compositions useful for treating or preventing a fibrotic disease or disorder in a subject, for example, comprising an antibody, inhibitor(s) of epiregulin activity, and antigen-binding fragment disclosed herein, are also disclosed.

### Definitions

Unless specifically indicated otherwise, all technical and scientific terms used herein have the same meaning as commonly understood by those of ordinary skill in the art to which this application belongs. In addition, any method or material similar or equivalent to a method or material described herein can be used in the practice of the present application. For purposes of the present application, the following terms are defined.

It is understood that embodiments of the application described terms of “comprising” herein include “consisting of” and/or “consisting essentially of” embodiments.

The term “antibody” refers to all isotypes of immunoglobulins (e.g., IgG, IgA, IgE, IgM, IgD, and IgY) including various monomeric, polymeric and chimeric forms, unless otherwise specified. Specifically encompassed by the term “antibody” are polyclonal antibodies, monoclonal antibodies (mAbs), and antibody-like polypeptides, such as chimeric antibodies and humanized antibodies. Immunoglobulin molecules can be of any class (e.g., IgG1, IgG2, IgG3, IgG4, IgM1, IgM2, IgA1 and IgA2) or subclass.

The term “antigen-binding fragment” refers to any proteinaceous structure that may exhibit binding affinity for a particular antigen. Antigen-binding fragments include those produced by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more complementarity determining regions (CDRs) of an antibody known to bind a particular antigen. Examples of suitable antigen-binding fragments include, but not limited to, single-chain molecules such as Fab, F(ab)2, Fc, Fabc, Fv molecules, scFv, and disulfide-linked Fvs (sdFv), intrabodies, diabodies, minibodies, linear antibodies, single domain antibodies such as sdAb (either VL or VH), camelid nanobodies (VHH domains), multi-specific antibodies formed from antibody fragments, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CH1 domains, or a monovalent antibody as described in WO2007059782 (which is incorporated herein by reference in its entirety), bivalent fragments comprising two Fab fragments linked by a disulfide bridge at the hinge region, a Fd fragment consisting essentially of the VH and CH1 domains, a dAb fragment, or an isolated CDR, and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. The phrase “an antibody or antigen-binding fragment thereof” may be used to denote that a given antigen-binding fragment incorporates one or more amino acid segments of the antibody referred to in the phrase.

The term “epitope” refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of nonlinear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The terms “polypeptide” or “peptide” are used herein to encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “percent (%) sequence identity” or “homology” with respect to the polypeptide and nucleotide sequences described herein is defined as the percentage of amino acid or nucleic acid residues in a candidate sequence that are identical with the amino acid or nucleic acid residues in the reference sequence being compared after aligning the sequences. In some cases, conservative substitutions are considered as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32 (5): 1792-1797, 2004; Edgar. R. C., BMC Bioinformatics 5 (1): 113, 2004, each of which are incorporated herein by reference in their entirety for all purposes).

The term “chimeric antigen receptor” or “CAR” as used herein refers to a cell-surface receptor comprising an extracellular antigen-binding domain, a transmembrane domain, and a cytoplasmic domain comprising a lymphocyte activation domain and optionally a co-stimulatory signaling domain(s), all in a combination that is not found together in nature on a single. This includes, without limitation, receptors wherein the extracellular domain and the cytoplasmic domain are not found together in nature on a single receptor protein. The chimeric antigen receptors of the present disclosure can be used with lymphocytes such as T-cells and natural killer (NK) cells.

The terms “T cell” and “T lymphocyte” are interchangeable and used synonymously herein. Non-limiting examples of T cells include naive T lymphocytes, thymocytes, mature T lymphocytes, immature T lymphocytes, activated T lymphocytes, or resting T lymphocytes. A T cell may be a T helper (Th) cell, e.g., a T helper 1 (Th1) or a T helper 2 (Th2) cell. The T cell may be a cytotoxic T cell (CTL; CD8+ T cell), a helper T cell (HTL; CD4+ T cell) CD4+T cell, CD4+CD8+ T cell, a tumor infiltrating cytotoxic T cell (TIL; CD8+ T cell), or any other subclass of T cells. Additional exemplary populations of T cells include memory T cells and naive T cells. Further included are “NKT cells”, which comprise a specialized population of T cells that express a semi-invariant αβ T-cell receptor, as well as various molecular markers that are commonly associated with natural killer cells (NK cells), e.g., NK1.1. NKT cells include and NK1.1⁻ and NK1.1⁺, as well as, CD4⁻, CD4⁺, CD8⁻ and CD8⁺ cells. The T cell receptor (TCR) on NKT cells is distinct insofar as it recognizes glycolipid antigens presented by the Major Histocompatibility Complex I (MHC-I)-like molecule CD Id. Also included are “Gamma-delta T cells (γδ T cells)” which may refer to a specialized subset of T cells possessing a distinct TCR on their surface, and unlike most T cells in which the TCR is comprised of two glycoprotein chains called α- and β-TCR chains, the TCR in γδ T cells is comprised of a γ-chain and a δ-chain. γδ T cells can contribute to immunosurveillance and immunoregulation, and may be a key source of IL-17 and induce a strong CD8+ cytotoxic T cell response. Also included are “regulatory T cells” or “Tregs”, which refer to T cells that suppress an abnormal or excessive immune response and contribute to immune tolerance. Tregs are commonly transcription factor Foxp3-positive CD4+ T cells and can also comprise transcription factor Foxp3-negative regulatory T cells that are IL-10-producing CD4+ T cells.

As used herein, the term “host cell” can be any type of cell, e.g., a primary cell, a cell in culture, or a cell from a cell line. In specific embodiments, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

As use herein, the terms “specifically binds,” “binds specifically.” “specifically recognizing,” and “is specific for” or derivatives thereof when used in the context of antibodies, or antibody fragments, means the antibody or antibody fragment forms a complex with an antigen (e.g., epiregulin) that is relatively stable under physiologic conditions. In certain embodiments, specific binding is determinative of the presence of the target in the presence of a heterogeneous population of molecules, including biological molecules (e.g., cell surface receptors). For example, an antibody that specifically recognizes a target (which can be an epitope) is an antibody that binds this target with greater affinity, avidity, more readily, and/or with greater duration than its bindings to other molecules. In some embodiments, the extent of binding of an antibody to an unrelated molecule is less than about 10% of the binding of the antibody to the target as measured, e.g., by a radioimmunoassay (RIA). In some embodiments, an antibody that specifically binds a target has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA, RIA, ECL, IRMA, EIA, BIACORE™ bio-layer interferometry assays, and peptide scans.

As used herein, the term “inhibit” means to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be, for example, a 10%, 20%, 30%, 40%, 50%, 60%, 70%, 80%, 90%, 100%, or any amount of reduction in between as compared to native or control levels.

As used herein. “treatment” or “treating” is an approach for obtaining beneficial or desired results including clinical results. For purposes of this application, beneficial or desired clinical results include, but are not limited to, one or more of the following: alleviating one or more symptoms resulting from the disease or disorder, diminishing the extent of the disease or disorder, stabilizing the disease or disorder (e.g., preventing or delaying the worsening of the disease or disorder), preventing or delaying the spread of the disease or disorder, preventing or delaying the recurrence of the disease or disorder, delay or slowing the progression of the disease or disorder, ameliorating the disease or disorder state, providing a remission (partial or total) of the disease or disorder, decreasing the dose of one or more other medications required to treat the disease or disorder, increasing the quality of life, and/or prolonging survival. Also encompassed by “treatment” is a reversal of one or more pathological consequences of the disease or disorder. As used herein, the term “reversal” or “reverse” in the context of a pathological consequence(s) of a disease or disorder may refer to the total reversal of a pathological consequence(s) of the disease or disorder (e.g., complete restoration to the non-diseased state), and to the partial reversal of the pathological consequence(s) (e.g., a reduction by any amount in the severity of the pathological consequence(s)). The methods of the application contemplate any one or more of these aspects of treatment. The benefit to a subject to be treated is either statistically significant or at least perceptible to the patient or to the physician.

The term “fibrosis” is well known in the art and is used herein to refer to the formation or development or accumulation of excess extracellular matrix in an organ or tissue as a reparative or reactive process, as opposed to a formation of healthy tissue as a normal constituent of an organ or tissue.

The term “effective amount” used herein refers to an amount of an agent or composition sufficient to treat a specified state, disorder, condition, or disease such as ameliorate, palliate, lessen, and/or delay one or more of its symptoms (e.g., clinical or sub-clinical symptoms). For therapeutic use, beneficial or desired results include, e.g., decreasing one or more symptoms resulting from the disease (biochemical, histologic and/or behavioral), including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients. An effective amount can be administered in one or more administrations. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration or other state/federal government or listed in the U.S. Pharmacopeia or other generally recognized pharmacopeia for use in mammals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin, incorporated by reference in its entirety for all purposes.

The terms “subject,” “individual,” and “patient” are used interchangeably herein to refer to a mammal, including, but not limited to, human, bovine, horse, feline, canine, rodent, or primate. In some embodiments, the subject is a human. In a preferred embodiment, the subject is a human.

Reference to “about” a value or parameter herein includes (and describes) variations that are directed to that value or parameter per se. For example, description referring to “about X” includes description of “X”. In certain embodiments, a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “about X-Y” used herein has the same meaning as “about X to about Y.”

As used herein and in the appended claims, the singular forms “a,” “an,” “or,” and “the” include plural referents unless the context clearly dictates otherwise. Thus, for example, a reference to “a method” includes one or more methods, and/or steps of the type described herein and/or which will become apparent to those persons skilled in the art upon reading this disclosure. As is apparent to one skilled in the art, a subject assessed, selected for, and/or receiving treatment is a subject in need of such activities.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor. New York; Ausubel et al, eds. (2005) Current Protocols in Molecular Biology. John Wiley and Sons, Inc.: Hoboken. NJ; Bonifacino et al, eds. (2005) Current Protocols in Cell Biology. John Wiley and Sons. Inc.: Hoboken, NJ; Coligan et al, eds. (2005) Current Protocols in Immunology, John Wiley and Sons, Inc.: Hoboken, NJ; Coico et al, eds. (2005) Current Protocols in Microbiology, John Wiley and Sons, Inc.: Hoboken, NJ; Coligan et al, eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437, each of which is incorporated by reference in their entirety for all purposes.

The terms and expressions which have been employed are used as terms of description and not of limitation, and use of such terms and expressions do not exclude any equivalents of the features shown and described or portions thereof, and various modifications are possible within the scope of the technology claimed.

### Epiregulin Inhibitors

Inhibiting epiregulin activity can be accomplished using any method known to the skilled artisan. Examples of methods to inhibit epiregulin activity include, but are not limited to decreasing expression of an endogenous epiregulin gene, decreasing expression of epiregulin mRNA, and inhibiting activity of epiregulin protein. An epiregulin inhibitor may therefore be a compound or composition that decreases expression of an epiregulin gene, a compound or composition that decreases epiregulin mRNA half-life, stability and/or expression, or a compound or composition that inhibits epiregulin protein function. Non-limiting examples of an epiregulin inhibitor that may be used in accordance with the present disclosure include, without limitation, an antibody or an antigen-binding fragment, a small molecule, a decoy receptor (e.g., a soluble receptor), a CAR modified cell (e.g., a CAR-T cell), an aptamer, an alternative scaffold, a polypeptide, a nucleic acid, an siRNA, a ribozyme, an antisense molecule, a peptidomimetic, or any combination thereof.

Epiregulin inhibition may be accomplished either directly or indirectly. For example, epiregulin may be directly inhibited by compounds or compositions that directly interact with epiregulin protein, such as antibodies or soluble epiregulin receptors. Alternatively, epiregulin may be inhibited indirectly by compounds or compositions that inhibit epiregulin receptors, epiregulin downstream effectors, or upstream regulators which up-regulate epiregulin expression.

Decreasing expression of an endogenous epiregulin gene includes providing a specific inhibitor of epiregulin gene expression. Decreasing expression of epiregulin mRNA or epiregulin protein includes decreasing the half-life or stability of epiregulin mRNA or decreasing expression of epiregulin mRNA. Methods of decreasing expression of epiregulin include, but are not limited to, methods that use an siRNA, a microRNA, an antibody, a soluble receptor, an antisense nucleic acid, a ribozyme, an expression vector encoding a transdominant negative mutant, a peptide, a small molecule, other specific inhibitors of epiregulin gene, mRNA, and protein expression, and combinations thereof.

### Antibodies and Antigen-Binding Fragments

In one embodiment, the epiregulin inhibitor is an antibody or an antigen-binding fragment. It will be appreciated by one skilled in the art that an antibody comprises any immunoglobulin molecule, whether derived from natural sources or from recombinant sources, which is able to specifically bind to an epitope present on a target molecule. In the present disclosure, the target molecule may be epiregulin, an epiregulin receptor, an epiregulin downstream effector, or fragments thereof. In one aspect of the disclosure, epiregulin is directly inhibited by an antibody or an antigen-binding fragment that specifically binds to an epitope on epiregulin. In another aspect, epiregulin is indirectly inhibited by an antibody or an antigen-binding fragment that specifically binds to an epitope on an epiregulin receptor. In yet another aspect, the effects of epiregulin are blocked by an antibody or an antigen-binding fragment that specifically binds to an epitope on a downstream effector such as extracellular matrix (ECM) proteins, proteases, anti-proteases, transcription factors, fibrogenetic cytokines, apoptosis regulators.

In some embodiments, described herein are isolated antibody, or an antigen-binding fragment thereof, that specifically binds to epiregulin. In some embodiments, the epiregulin is human epiregulin. In some embodiments, the isolated antibody or antigen-binding fragment specifically described herein binds to an epidermal growth factor (EGF)-like domain of epiregulin.

In some embodiments, an isolated antibody or antigen-binding fragment described herein specifically binds to epiregulin with high affinity, for example, a K_{D} of less than about 1×10⁻⁸ M, such as but not limited to, about 1-9.9 (or any range or value therein, such as 1, 2, 3, 4, 5, 6, 7, 8, or 9×10⁻⁹ M, 10⁻¹⁰ M, 10⁻¹¹ M, 10⁻¹² M, 10⁻¹³ M, 10⁻¹⁴ M, 10⁻¹⁵ M or any range or value therein, as determined by, for example, bio-layer interferometry assay, surface plasmon resonance, or the Kinexa method, as practiced by those of skill in the art. In some embodiments, the K_{D} is equal to or less than 1×10⁻⁹ M. In some embodiments, the K_{D} is equal to or less than 1×10⁻¹⁰ M. In some embodiments, the K_{D} is about 1× 10⁻¹¹, 2×10⁻¹¹. 3×10⁻¹¹, 4×10⁻¹¹, 5×10⁻¹¹, 6×10⁻¹¹, 7×10⁻¹¹, 8×10⁻¹¹, 9×10⁻¹¹, 1×10⁻¹⁰, 2×10⁻¹⁰, 3×10⁻¹⁰, 4×10⁻¹⁰, 5×10⁻¹⁰, 6×10⁻¹⁰, 7×10⁻¹⁰, 8×10⁻¹⁰, or 9× 10⁻¹⁰ M. One example K_{D} is equal to about 3.8×10⁻¹¹ M.

Methods of testing antibodies for the ability to bind to the target peptide or any portion thereof are known in the art and include any antibody-antigen binding assay, such as, for example, bio-layer interferometry assay, radioimmunoassay (RIA), Western blot, enzyme-linked immunosorbent assay (ELISA), immunoprecipitation, and competitive inhibition assays. In some embodiments, K_{D} values described herein are determined using a bio-layer interferometry assay.

In some embodiments, an isolated antibody or antigen-binding fragment described herein is an epiregulin neutralizing antibody or antigen-binding fragment. In some embodiments, the isolated antibody or antigen-binding fragment described herein inhibits an activity of epiregulin. In some embodiments, the isolated antibody or antigen-binding fragment described herein inhibits epiregulin's interaction with an ErbB receptor. In some embodiments, the ErbB receptor is epidermal growth factor receptor (EGFR), human epidermal growth factor receptor 2 (HER2), HER3, and/or HER4. In some embodiments, the ErbB receptor is EGFR.

In some embodiments, the isolated antibody or antigen-binding fragment described herein inhibits epiregulin-induced proliferation of a fibroblast. In some embodiments, the isolated antibody or antigen-binding fragment described herein inhibits epiregulin-induced proliferation of a fibroblast with an IC₅₀ of less than about 100 nM, such as but not limited to, about 90 nM, 80 nM. 70 nM, 60 nM, 50 nM, 40 nM, 30 nM, 20 nM, 10 nM, 9 nM, 8 nM, 7 nM, 6 nM, 5 nM, 4 nM, 3 nM, 2.5 nM, 2 nM, 1.5 nM, 1 nM, 0.5 nM, 0.2 nM, or 0.1 nM. In some embodiments, the isolated antibody or antigen-binding fragment described herein inhibits epiregulin-induced proliferation of a fibroblast with an IC₅₀ of less than about 10 nM. One example IC₅₀ is equal to or less than about 10 nM. One example IC₅₀ is equal to about 1.8 nM.

In some embodiments, the isolated antibody or antigen-binding fragment described herein does not specifically bind to mouse epiregulin.

In some embodiments, the isolated antibody or antigen-binding fragment described herein does not specifically bind to one or more other human EGFR ligands. In some embodiments, the one or more other EGFR ligands are transforming growth factor-alpha (TGFA), betacellulin (BTC), heparin-binding EGF-like growth factor (HB-EGF), epigen (EPGN), epidermal growth factor (EGF), and/or amphiregulin (AREG).

In some embodiments, the isolated antibody or antigen-binding fragment described herein comprises three heavy chain CDRs (HCDR1, HCDR2 and HCDR3) contained within a heavy chain variable region (VH) comprising the amino acid sequence of SEQ ID NO: 1, or a sequence having at least 70%, 75%, 80%, 85%, 90%, 91%, 92%, 93%, 94%, 95%, 96%, 97%, 98%, or 99% identity thereto.

In some embodiments, the isolated antibody or antigen-binding fragment described herein comprises three heavy chain CDRs (HCDR1, HCDR2 and HCDR3) contained within a heavy chain variable region (VH) encoded by the nucleotide sequence of SEQ ID NO: 5, or a sequence having at least 70%, 75%, 80%, 85%, 90%, 91%, 92%, 93%, 94%, 95%, 96%, 97%, 98%, or 99% identity thereto.

In some embodiments, the isolated antibody or antigen-binding fragment described herein comprises an HCDR1 comprising the amino acid sequence of GGSISSSGYY (SEQ ID NO: 2), an HCDR2 comprising the amino acid sequence of FYYSGNT (SEQ ID NO: 3), and/or an HCDR3 comprising the amino acid sequence of ARHPFNWNDHYHYMDV (SEQ ID NO: 4).

Amino acid sequence of hEreg NAb1 heavy chain variable region (VH), HCDR sequences are bolded and underlined

(SEQ ID NO: 1)
HLQLQESGPGLVKSSETLSLTCSVSGGSISSSGYYWGWIRQPPGKGLEW
IGSFYYSGNTYYNPSLKSRVTISADTSKSQYSLKLSSVTAADTAVYYCA
RHPFNWNDHYHYMDVWGNGTTVTVSS
Nucleotide sequence encoding hEreg NAb1 heavy
chain variable region (VH)
(SEQ ID NO: 5)
CACCTGCAACTGCAGGAGTCGGGCCCAGGACTGGTGAAGTCTTCGGAGA
CCCTGTCCCTCACCTGCTCTGTCTCTGGTGGCTCCATCAGCAGTAGTGG
TTACTACTGGGGCTGGATCCGCCAGCCCCCAGGGAAGGGCCTGGAGTGG
ATTGGGAGTTTCTATTATAGTGGGAACACCTACTACAACCCGTCCCTCA
AGAGTCGAGTCACCATATCCGCAGACACGTCCAAGAGCCAGTACTCCCT
GAAGCTGAGTTCTGTGACCGCCGCAGACACGGCTGTGTATTATTGTGCG
AGACATCCGTTCAACTGGAACGACCACTATCACTACATGGACGTCTGGG
GCAACGGGACCACGGTCACCGTCTCCTCA

In some embodiments, the isolated antibody or antigen-binding fragment described herein comprises three light chain CDRs (LCDR1, LCDR2 and LCDR3) contained within a light chain variable region (VL) comprising the amino acid sequence of SEQ ID NO: 6, or a sequence having at least 70%, 75%, 80%, 85%, 90%, 91%, 92%, 93%, 94%, 95%, 96%, 97%, 98%, or 99% identity thereto.

In some embodiments, the isolated antibody or antigen-binding fragment described herein comprises three light chain CDRs (LCDR1, LCDR2 and LCDR3) contained within a light chain variable region (VL) encoded by the nucleotide sequence of SEQ ID NO: 10, or a sequence having at least 70%, 75%, 80%, 85%, 90%, 91%, 92%, 93%, 94%, 95%, 96%, 97%, 98%, or 99% identity thereto.

In some embodiments, the isolated antibody or antigen-binding fragment described herein comprises a LCDR1 comprising the amino acid sequence of QSISNY (SEQ ID NO: 7), a LCDR2 comprising the amino acid sequence of AAS (SEQ ID NO: 8), and/or a LCDR3 comprising the amino acid sequence of QQSYITSIT (SEQ ID NO: 9).

Amino acid sequence of hEreg NAb1 light chain variable region (VL), LCDR sequences are bolded and underlined

(SEQ ID NO: 6)
DIQMTQSPSSLSASVGDSVTITCRASQSISNYLNWYQKKPGKAPKVLIY
AASSLQSGVPSRFSGSGSGTDFTLTISSLQPEDFATYYCQQSYITSITF
GQGTRLEIK
Nucleotide sequence encoding hEreg NAb1 light
chain variable region (VL)
(SEQ ID NO: 10)
GACATCCAGATGACCCAGTCTCCATCCTCCCTGTCTGCATCTGTGGGAG
ACAGCGTCACCATCACTTGCCGGGCAAGTCAGAGCATTAGCAACTATTT
AAATTGGTATCAGAAGAAACCAGGGAAAGCCCCTAAGGTCCTGATCTAT
GCTGCATCCAGTTTGCAAAGTGGAGTCCCATCAAGGTTCAGTGGCAGTG
GATCTGGAACAGATTTCACTCTCACCATCAGCAGTCTGCAACCTGAAGA
TTTTGCAACTTACTACTGTCAACAGAGTTACATAACCTCGATCACCTTC
GGCCAAGGGACACGACTGGAGATTAAA

In some embodiments, the isolated antibody or antigen-binding fragment described herein comprises three heavy CDRs (HCDR1, HCDR2 and HCDR3) contained within a VH comprising the amino acid sequence of SEQ ID NO: 1, or a sequence having at least 70%, 75%, 80%, 85%, 90%, 91%, 92%, 93%, 94%, 95%, 96%, 97%, 98%, or 99% identity thereto; and/or three light chain CDRs (LCDR1, LCDR2 and LCDR3) contained within a VL comprising the amino acid sequence of SEQ ID NO: 6, or a sequence having at least 70%, 75%, 80%, 85%, 90%, 91%, 92%, 93%, 94%, 95%, 96%, 97%, 98%, or 99% identity thereto.

In some embodiments, the isolated antibody or antigen-binding fragment described herein comprises three heavy CDRs (HCDR1, HCDR2 and HCDR3) contained within a VH comprising the amino acid sequence of SEQ ID NO: 1, or a sequence having at least 70%, 75%, 80%, 85%, 90%, 91%, 92%, 93%, 94%, 95%, 96%, 97%, 98%, or 99% identity thereto; and three light chain CDRs (LCDR1, LCDR2 and LCDR3) contained within a VL comprising the amino acid sequence of SEQ ID NO: 6, or a sequence having at least 70%, 75%, 80%, 85%, 90%, 91%, 92%, 93%, 94%, 95%, 96%, 97%, 98%, or 99% identity thereto.

In some embodiments, the isolated antibody or antigen-binding fragment described herein comprises three heavy CDRs (HCDR1, HCDR2 and HCDR3) contained within a VH comprising the amino acid sequence of SEQ ID NO: 1; and/or three light chain CDRs (LCDR1, LCDR2 and LCDR3) contained within a VL comprising the amino acid sequence of SEQ ID NO: 6.

In some embodiments, the isolated antibody or antigen-binding fragment described herein comprises three heavy CDRs (HCDR1, HCDR2 and HCDR3) contained within a VH comprising the amino acid sequence of SEQ ID NO: 1; and three light chain CDRs (LCDR1, LCDR2 and LCDR3) contained within a VL comprising the amino acid sequence of SEQ ID NO: 6.

In some embodiments, the isolated antibody or antigen-binding fragment described herein comprises an HCDR1 comprising the amino acid sequence of SEQ ID NO: 2, an HCDR2 comprising the amino acid sequence of SEQ ID NO: 3, and/or an HCDR3 comprising the amino acid sequence of SEQ ID NO: 4; and/or a LCDR1 comprising the amino acid sequence of SEQ ID NO: 7, a LCDR2 comprising the amino acid sequence of SEQ ID NO: 8, and/or a LCDR3 comprising the amino acid sequence of SEQ ID NO: 9.

In some embodiments, the isolated antibody or antigen-binding fragment described herein comprises an HCDR1 comprising the amino acid sequence of SEQ ID NO: 2, an HCDR2 comprising the amino acid sequence of SEQ ID NO: 3, and an HCDR3 comprising the amino acid sequence of SEQ ID NO: 4; and a LCDR1 comprising the amino acid sequence of SEQ ID NO: 7, a LCDR2 comprising the amino acid sequence of SEQ ID NO: 8, and a LCDR3 comprising the amino acid sequence of SEQ ID NO: 9.

In some embodiments, the isolated antibody or antigen-binding fragment described herein comprises a VH comprising the amino acid sequence of SEQ ID NO: 1, or a sequence having at least 70%, 75%, 80%, 85%, 90%, 91%, 92%, 93%, 94%, 95%, 96%, 97%, 98%, or 99% identity thereto; and/or a VL comprising the amino acid sequence of SEQ ID NO: 6, or a sequence having at least 70%, 75%, 80%, 85%, 90%, 91%, 92%, 93%, 94%, 95%, 96%, 97%, 98%, or 99% identity thereto.

In some embodiments, the isolated antibody or antigen-binding fragment described herein comprises a VH comprising the amino acid sequence of SEQ ID NO: 1, or a sequence having at least 70%, 75%, 80%, 85%, 90%, 91%, 92%, 93%, 94%, 95%, 96%, 97%, 98%, or 99% identity thereto; and a VL comprising the amino acid sequence of SEQ ID NO: 6, or a sequence having at least 70%, 75%, 80%, 85%, 90%, 91%, 92%, 93%, 94%, 95%, 96%, 97%, 98%, or 99% identity thereto.

In some embodiments, the isolated antibody or antigen-binding fragment described herein comprises a VH comprising the amino acid sequence of SEQ ID NO: 1, and/or a VL comprising the amino acid sequence of SEQ ID NO: 6.

In some embodiments, the isolated antibody or antigen-binding fragment described herein comprises a VH comprising the amino acid sequence of SEQ ID NO: 1, and a VL comprising the amino acid sequence of SEQ ID NO: 6.

In some embodiments, the isolated antibody or antigen-binding fragment described herein is a human antibody, a monoclonal antibody, a humanized antibody, a single chain antibody, a Fab, a Fab′, a F(ab′)2, a Fv, or a scFv.

A skilled artisan will understand that the exact definitional CDR boundaries and lengths are subject to different classification and numbering systems, CDRs may therefore be referred to by Kabat, Chothia, contact or any other boundary definitions. Despite differing boundaries, each of these systems has some degree of overlap in what constitutes the so called “hypervariable regions” within the variable sequences, CDR definitions according to these systems may therefore differ in length and boundary areas with respect to the adjacent framework region. See for example Kabat et al., NIH Publication No. 91-3242 (1991); Chothia et al., J. Mol. Biol. 196:901 (1987); and MacCallum et al., J. Mol. Biol. 262:732 (1996)), each of which is hereby incorporated by reference in its entirety.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three-dimensional structures, despite high amino acid sequence variability in most parts of the loops (Chothia et al., J. Mol. Biol. 196:901 (1987); Chothia et al., “Conformations of Immunoglobulin Hypervariable Regions,” Nature. 342:877 (1989); Martin and Thornton, J. Mol. Biol. 263:800 (1996), each of which is incorporated by reference in its entirety). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

Also disclosed herein are isolated antibodies or antigen-binding fragments thereof that compete for binding to epiregulin with the anti-epiregulin antibody or antigen-binding fragment described herein. The term “competes” or “cross-competes”, as used herein, means an antibody or antigen-binding fragment thereof binds to an antigen and inhibits or blocks the binding of another antibody or antigen-binding fragment thereof. The term also includes competition between two antibodies in both orientations (wherein a first antibody that binds and blocks binding of the second antibody and vice-versa). In some embodiments, a competing antibody and an anti-epiregulin antibody described herein may bind to the same epitope. Alternatively, a competing antibody and an anti-epiregulin antibody described herein may bind to different, but overlapping epitopes such that binding of one inhibits or blocks the binding of the second antibody, e.g., via steric hindrance. Cross-competition between antibodies may be measured by methods known in the art, for example, by a real-time, label-free bio-layer interferometry assay. Cross-competition between two antibodies may be expressed as the binding of the second antibody that is less than the background signal due to self-self binding (wherein first and second antibodies is the same antibody). Cross-competition between two antibodies may be expressed, for example, as % binding of the second antibody that is less than the baseline self-self background binding (wherein first and second antibodies is the same antibody).

Also disclosed herein are isolated antibodies or antigen-binding fragments thereof that bind to the same epitope as the anti-epiregulin antibody or antigen-binding fragment described herein.

Without wishing to be bound by theory, when the epiregulin inhibitor used in the compositions and methods described herein is a polyclonal antibody, the antibody may be generated by inoculating a suitable animal with a peptide comprising epiregulin, an epiregulin receptor, an epiregulin downstream effector, or fragments thereof. These polypeptides, or fragments thereof, may be obtained by any method known in the art, including chemical synthesis and biological synthesis. Antibodies produced in the inoculated animal which specifically bind to epiregulin, an epiregulin receptor, an epiregulin downstream effector, or fragments thereof, are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1988, In: Antibodies, A Laboratory Manual, Cold Spring Harbor, N.Y.).

Monoclonal antibodies directed against epiregulin, an epiregulin receptor, an epiregulin downstream effector, or fragment thereof, may be prepared using any well-known monoclonal antibody preparation procedures, such as those described, for example, in Harlow et al. (1988, In: Antibodies. A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Monoclonal antibodies directed against an antigen may be generated from mice immunized with the antigen using standard procedures known in the art. Nucleic acid encoding the monoclonal antibody obtained using such procedures may be cloned and sequenced using technology which is available in the art, and is described, for example, in Wright et al. (1992, Critical Rev. in Immunol. 12 (3, 4): 125-168) and the references cited therein.

When the antibody used in the methods of the disclosure is a biologically active antibody fragment or a synthetic antibody which may target an epiregulin, an epiregulin receptor, an epiregulin downstream effector, or fragments thereof, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology, which is available in the art, and described, for example, in Wright et al. (1992, Critical Rev. in Immunol. 12 (3, 4): 125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

A non-limiting example of an antibody or an antigen-binding fragment which may be used in accordance with the disclosure comprises an anti-human epiregulin antibody AF1195 which can be purchased from R&D Biosciences. Another non-limiting example of an antibody or an antigen-binding fragment which may be used in accordance with the disclosure comprises anti-mouse/human epiregulin antibody Clone #189611 which can be purchased from R&D Biosciences (MAB1068).

In some embodiments, the isolated antibody or antigen-binding fragment described herein is a humanized antibody. The present disclosure also includes use of humanized antibodies specifically reactive with an epitope present on the target molecule (e.g., epiregulin). Humanized forms of non-human (e.g., murine) antibodies include chimeric immunoglobulins, immunoglobulin chains or fragments thereof which contain minimal sequence derived from non-human immunoglobulin. Humanized antibodies include human immunoglobulins (recipient antibody) in which residues from a CDR of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also contain residues that are found neither in the recipient antibody nor in the imported CDR or framework sequences. In general, the humanized antibody will contain substantially all of at least one, and typically two, variable domains, in which all or substantially all of the CDR regions correspond to those of a non-human immunoglobulin and all or substantially all of the framework regions are those of a human immunoglobulin consensus sequence. A humanized antibody can optimally contain at least a portion of an immunoglobulin constant region (Fc), typically that of a human immunoglobulin.

Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed by substituting rodent CDRs or CDR sequences for the corresponding sequences of a human antibody. Accordingly, a humanized form of a non-human antibody (or a fragment thereof) is a chimeric antibody or fragment, wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework for the humanized antibody. Another method uses a particular framework derived from the consensus sequence of all human antibodies of a particular subgroup of light or heavy chains. The same framework may be used for several different humanized antibodies.

It is further important that antibodies be humanized with retention of high affinity for the antigen and other favorable biological properties. To achieve this goal, humanized antibodies are preferably prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework residues can be selected and combined from the consensus and import sequence so that the desired antibody characteristic, such as increased affinity for the target antigen(s), is achieved. In general, the CDR residues are directly and most substantially involved in influencing antigen binding.

The antibodies or antigen-binding fragments described herein include variants having single or multiple amino acid substitutions, deletions, or additions that retain the biological properties (e.g., binding affinity or immune effector activity) of the described antibodies or antigen-binding fragments.

These variants may include: (i) variants in which one or more amino acid residues are substituted with conservative or nonconservative amino acids. (ii) variants in which one or more amino acids are added to or deleted from the polypeptide, (iii) variants in which one or more amino acids include a substituent group, and (iv) variants in which the described antibody or antigen-binding fragment is fused or conjugated with another peptide or polypeptide (e.g., a fusion partner, a protein tag) or other chemical moiety, that may confer useful properties to the antibody or antigen-binding fragment, such as, for example, an epitope for an antibody, a polyhistidine sequence, a biotin moiety and the like. Antibodies or antigen-binding fragments described herein may include variants in which amino acid residues from one species are substituted for the corresponding residue in another species, either at the conserved or nonconserved positions. In other embodiments, amino acid residues at nonconserved positions are substituted with conservative or nonconservative residues. Amino acid substitutions are generally based on the relative similarity of the amino acid side-chain substituents, for example, their hydrophobicity, hydrophilicity, charge, size, and the like.

Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group I (Ala, Ser, Thr, Pro, Gly); Group II (Asp, Asn, Glu, Gln); Group III (His, Arg, Lys); Group IV (Met, Leu, Ile, Val, Cys); Group V (Phe, Thy, Trp).

Accordingly, embodiments of the antibody or antigen-binding fragment can include variants having about 50%, 60%, 70%, 80%, 90%, 91%, 92%, 93%, 94%, 95%, 96%, 97%, 98%, 99%, or more sequence identity to the described antibody or antigen-binding fragment.

The antibodies or antigen-binding fragments described herein may of any one of various antibody isotypes, such as IgM, IgD, IgG, IgA and IgE. In some embodiments, the antibody isotype is IgG1, IgG2, IgG3, or IgG4 isotype. In some embodiments, the antibody isotype is IgA1 or IgA2. Antibody or antigen-binding fragment thereof specificity is largely determined by the amino acid sequence, and arrangement, of the CDRs. Therefore, the CDRs of one isotype may be transferred to another isotype without altering antigen specificity. Alternatively, techniques have been established to cause hybridomas to switch from producing one antibody isotype to another (isotype switching) without altering antigen specificity. Accordingly, such antibody isotypes are within the scope of the described antibodies or antigen-binding fragments.

One of skill in the art will further appreciate that the present disclosure encompasses the use of antibodies derived from camelid species. That is, the present disclosure includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1988, Antibodies: A Laboratory Manual, Cold Spring Harbor, N.Y.).

V_{H} proteins isolated from other sources, such as animals with heavy chain disease (Seligmann et al., 1979, Immunological Rev. 48:145-167, incorporated herein by reference in its entirety), are also useful in the compositions and methods of the disclosure. The present disclosure further comprises variable heavy chain immunoglobulins produced from mice and other mammals, as detailed in Ward et al. (1989, Nature 341:544-546, incorporated herein by reference in its entirety). Briefly, V_{H} genes are isolated from mouse splenic preparations and expressed in E, coli. The present disclosure encompasses the use of such heavy chain immunoglobulins in the compositions and methods detailed herein.

Antibodies useful as epiregulin inhibitors in the disclosure may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody, cDNA copies of the mRNA are produced using reverse transcriptase, cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the disclosure should not be construed to be limited solely to the generation of phage encoding Fab antibodies. Rather, phage which encode single chain antibodies (scFv/phage antibody libraries) are also included in the disclosure. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The disclosure should also be construed to include synthetic phage display libraries in which the heavy and light chain variable regions may be synthesized such that they include nearly all possible specificities (Barbas, 1995, Nature Medicine 1:837-839; de Kruif et al., 1995. J. Mol. Biol. 248:97-105).

Also disclosed are isolated polynucleotides that encode the antibodies or antigen-binding fragments described herein that specifically bind to epiregulin. The isolated polynucleotides capable of encoding the variable domain segments provided herein may be included on the same, or different, vectors to produce antibodies or antigen-binding fragments.

In some embodiments, isolated polynucleotides encoding an antibody or antigen-binding fragment described herein comprise a VH-encoding nucleotide sequence of SEQ ID NO: 5, or a sequence having at least 70%, 75%, 80%, 85%, 90%, 91%, 92%, 93%, 94%, 95%, 96%, 97%, 98%, or 99% identity thereto.

In some embodiments, isolated polynucleotides encoding an antibody or antigen-binding fragment described herein comprise a VL-encoding nucleotide sequence of SEQ ID NO: 10, or a sequence having at least 70%, 75%, 80%, 85%, 90%, 91%, 92%, 93%, 94%, 95%, 96%, 97%, 98%, or 99% identity thereto.

In some embodiments, isolated polynucleotides encoding an antibody or antigen-binding fragment described herein comprise a VH-encoding nucleotide sequence of SEQ ID NO: 5, or a sequence having at least 70%, 75%, 80%, 85%, 90%, 91%, 92%, 93%, 94%, 95%, 96%, 97%, 98%, or 99% identity thereto; and/or a VL-encoding nucleotide sequence of SEQ ID NO: 10, or a sequence having at least 70%, 75%, 80%, 85%, 90%, 91%, 92%, 93%, 94%, 95%, 96%, 97%, 98%, or 99% identity thereto.

In some embodiments, isolated polynucleotides encoding an antibody or antigen-binding fragment described herein comprise a VH-encoding nucleotide sequence of SEQ ID NO: 5, or a sequence having at least 70%, 75%, 80%, 85%, 90%, 91%, 92%, 93%, 94%, 95%, 96%, 97%, 98%, or 99% identity thereto; and a VL-encoding nucleotide sequence of SEQ ID NO: 10, or a sequence having at least 70%, 75%, 80%, 85%, 90%, 91%, 92%, 93%, 94%, 95%, 96%, 97%, 98%, or 99% identity thereto.

In some embodiments, isolated polynucleotides encoding an antibody or antigen-binding fragment described herein comprise a VH-encoding nucleotide sequence of SEQ ID NO: 5; and/or a VL-encoding nucleotide sequence of SEQ ID NO: 10.

In some embodiments, isolated polynucleotides encoding an antibody or antigen-binding fragment described herein comprise a VH-encoding nucleotide sequence of SEQ ID NO: 5; and a VL-encoding nucleotide sequence of SEQ ID NO: 10.

Also disclosed are vectors comprising a polynucleotide encoding an antibody or antigen-binding fragment described herein.

Also disclosed are host cells expressing the recombinant antibody or antigen-binding fragment described herein. Such host cells may comprise a polynucleotide or a vector described above. In some embodiments, the host cell is a hybridoma. In some embodiments, the antibody or antigen-binding fragment is recombinantly produced.

Antibodies of the disclosure can be produced in a host cell transfectoma (a type of hybridoma) using, for example, a combination of recombinant DNA techniques and gene transfection methods as is well known in the art (e.g., Morrison, S. (1985) Science 229:1202).

For example, to express the antibodies, or antibody fragments thereof, polynucleotides encoding partial or full-length light and heavy chains, can be obtained by standard molecular biology techniques (e.g., PCR amplification or cDNA cloning using a hybridoma that expresses the antibody of interest) and the polynucleotides can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the described antibodies can be used to create full-length antibody genes of any antibody isotype by inserting them into expression vectors already encoding heavy chain constant and light chain constant regions of the desired isotype such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the disclosure carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, CA (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus. (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRD promoter system, which contains sequences from the SV40 early promoter and the long terminal repeat of human T cell leukemia virus type 1 (Takebe, Y, et al. (1988) Mol. Cell. Biol. 8:466-472).

In addition to the antibody chain genes and regulatory sequences, the recombinant expression vectors of the disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the disclosure in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A, and Wood, C. R. (1985) Immunology Today 6:12-13).

Suitable mammalian host cells for expressing the recombinant antibodies of the disclosure include Chinese Hamster Ovary (CHO cells) (including dhfr-CHO cells, described in Urlaub and Chasin, (1980) Proc. Natl. Acad, Sci. USA 77:4216-4220, used with a DHFR selectable marker, e.g., as described in R. J. Kaufman and P. A. Sharp (1982) Mol. Biol. 759:601-621), NSO myeloma cells, COS cells and SP2 cells. In particular, for use with NSO myeloma cells, another suitable expression system is the GS gene expression system disclosed in WO 87/04462. WO 89/01036 and EP 0338841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Once expressed, whole antibodies, dimers derived therefrom, individual light and heavy chains, antibody fragments, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Once purified, the antibodies may then be used to practice the method of the disclosure, or to prepare a pharmaceutical composition useful in practicing the method of the disclosure.

The antibodies or antigen-binding fragments of the present disclosure can be assayed for immunospecific binding by any method known in the art. The immunoassays which can be used include but are not limited to competitive and non-competitive assay systems using techniques such as western blots, radioimmunoassays. ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g. Current Protocols in Molecular Biology. (Ausubel et al., eds.). Greene Publishing Associates and Wiley-Interscience, New York (2002)). Exemplary immunoassays are described briefly below (but are not intended to be in any way limiting).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 14 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. Those of ordinary skill in the art will be knowledgeable as to the parameters that can be modified to increase the binding of the antibody to an antigen and decrease the background (e.g., pre-clearing the cell lysate with sepharose beads), upon consideration of the present disclosure. Additional immunoprecipitation protocols are presented Current Protocols in Molecular Biology, (Ausubel et al., eds.), Greene Publishing Associates and Wiley-Interscience. New York (2002).

Western blot analysis generally comprises preparing protein samples, electrophoresis of the protein samples in a polyacrylamide gel (e.g., about 8-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with about 3% BSA or non-fat milk), washing the membrane in washing buffer (e.g., PBS-TWEEN 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. Those of ordinary skill in the art will be knowledgeable as to the parameters that can be modified to increase the signal detected and to reduce the background noise, upon consideration of the present disclosure. Additional western blot protocols are presented in Current Protocols in Molecular Biology, (Ausubel et al., eds.), Greene Publishing Associates and Wiley-Interscience. New York (2002).

Enzyme-linked immunoassay (ELISAs) comprise preparing antigen, coating the well of a 96-well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. When performing an ELISA, the antibody of interest does not need to be conjugated to a detectable compound; instead, a second antibody (which recognizes the antibody of interest) conjugated to a detectable compound can be added to the well. Further, instead of coating the well with the antigen, the antibody may be coated to the well. In this case, a second antibody conjugated to a detectable compound can be added following the addition of the antigen of interest to the coated well. One of ordinary skill in the art will be knowledgeable as to the parameters that can be modified to increase the signal detected as well as other variations of ELISA protocols known in the art. For further discussion regarding ELISA protocols see, e.g., Current Protocols in Molecular Biology, (Ausubel et al., eds.). Greene Publishing Associates and Wiley-Interscience, New York (2002).

The binding affinity of an antibody to an antigen and the off-rate of an antibody-antigen interaction can be determined by competitive binding assays One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

### Decoy Receptors

In another embodiment of the disclosure, a decoy receptor such as, but not limited to, a soluble ErbB receptor (or a soluble fragment thereof), that binds to epiregulin is contemplated as an inhibitor of epiregulin activity. This agent can be used to reduce or prevent the binding of epiregulin to a cell-bound epiregulin receptor (e.g., EGFR) and thereby act as an antagonist of epiregulin activity. Such decoy receptors have been used to bind cytokines or other ligands to regulate their function (Thomson, (1998) Cytokine Handbook, Academic Press).

In some embodiments, a decoy receptor described herein is a soluble ErbB receptor, e.g., a soluble EGFR, a soluble ErbB-2, a soluble ErbB-3, a soluble ErbB-4, or a soluble fragment thereof. In some embodiments, a decoy receptor described herein is a soluble EGFR, or a soluble fragment thereof.

Without wishing to be bound by theory, decoy receptors may recognize certain molecules, e.g., growth factors (e.g., epiregulin), with high affinity and specificity, but are structurally incapable of signaling or presenting the agonist to signaling receptor complexes. They act as a molecular trap for the agonist and for signaling receptor components. The interleukin-1 type II receptor (IL-1RII) was the first identified pure decoy. Decoy receptors have subsequently been identified for members of, e.g., the tumor necrosis factor receptor and IL-1R families. Moreover, silent nonsignaling receptors could act as decoys. Therefore, the use of decoy receptors is a general strategy to regulate the action of various signaling molecules.

A decoy receptor may occur in solution, or outside of a membrane. Decoy receptors may occur because the segment of the molecule which spans or associates with the membrane is absent. This segment is commonly referred to in the art as the transmembrane domain of the receptor. Thus, in some embodiments of the disclosure, a soluble receptor includes a fragment or a variant of a membrane bound receptor which does not have the transmembrane domain. Preferably, the fragment contains at least 6, e.g., 10, 15, 20, 25, 30, 40, 50, 60, or 70 amino acids, provided it retains its desired activity (e.g., binding to epiregulin).

In other embodiments of the disclosure, the structure of the segment that associates with the membrane is modified (e.g., DNA sequence polymorphism or mutation in the gene) so the receptor is not tethered to the membrane, or the receptor is inserted, but is not retained within the membrane. Thus, a decoy receptor, in contrast to the corresponding membrane bound form, differs in one or more segments of the gene or receptor protein that are important to its association with the membrane.

The present disclosure encompasses cDNA encoding a decoy receptor (e.g., a soluble EGFR) which is isolated from decoy receptor-producing cells or is recombinantly engineered from decoy receptor-encoding DNA.

In some embodiments, decoy receptors (e.g., soluble ErbB receptors) described herein can bind to epiregulin without eliciting undesired downstream effects including, but not limited to skin rash, alopecia, pulmonary toxicity (e.g., pneumonitis or fibrosis), neuropathy, or gastrointestingal disturbance.

Any epiregulin receptor identified may serve as the basis for the generation of a decoy receptor of the present disclosure.

Any of a variety of procedures may be used to molecularly clone a decoy receptor (e.g., a soluble EGFR) described herein.

For example, suitable cDNA libraries may be prepared from cells or cell lines which have soluble receptor (e.g., a soluble EGFR) activity. The selection of cells or cell lines for use in preparing a cDNA library to isolate a soluble receptor (e.g., a soluble EGFR) cDNA may be done by first measuring receptor activity using a receptor binding assay.

Preparation of cDNA Libraries can be performed by standard techniques well known in the art. Well known cDNA library construction techniques can be found for example, in Maniatis, T., Fritsch, E. F., Sambrook, J., Molecular Cloning: A Laboratory Manual (Cold Spring Harbor Laboratory, Cold Spring Harbor, N.Y., 1982).

DNA encoding a decoy receptor may also be isolated from a suitable genomic DNA library. Construction of genomic DNA libraries can be performed by standard techniques well known in the art. Well known genomic DNA library construction techniques can be found in Maniatis, T., Fritsch. E. F., Sambrook, J, in Molecular Cloning: A Laboratory Manuel (Cold Spring Harbor Laboratory, Cold Spring Harbor, N.Y., 1982).

Decoy receptor molecules may also be obtained by recombinantly engineering them from DNA encoding the partial or complete amino acid sequence of the decoy receptor (e.g., soluble EGFR). Using recombinant DNA techniques, DNA molecules are constructed which encode at least a portion of the decoy receptor (e.g., soluble EGFR) capable of binding epiregulin without stimulating, pathological collagen deposition, and alveolar remodeling. Standard recombinant DNA techniques are used such as those found in Maniatis, et al., supra.

For purposes of illustration, DNA encoding a decoy receptor, for example, soluble EGFR, is utilized. Using the receptor DNA sequence, a DNA molecule is constructed which encodes the extracellular domain of the receptor, or the epiregulin binding domain only. Restriction endonuclease cleavage sites are identified within the receptor DNA and can be utilized directly to excise the extracellular-encoding portion. In addition, PCR techniques well known in the art may be utilized to produce the desired portion of DNA. Other cloning techniques may be utilized to produce decoy receptor molecules in a manner analogous to those described above.

The cloned decoy receptor (e.g., soluble EGFR) cDNA obtained through the methods described above may be recombinantly expressed by molecular cloning into an expression vector containing a suitable promoter and other appropriate transcription regulatory elements and transferred into prokaryotic or eukaryotic host cells to produce recombinant decoy receptor (e.g., soluble EGFR). Techniques for such manipulations are fully described in Maniatis, T, et al., supra, and are well known in the art.

### Chimeric Antigen Receptor (CAR)-Modified Cells

Chimeric antigen receptors (CARs) are hybrid molecules comprising an antigen-binding domain, commonly a single-chain variable fragment (scFv), followed by a linker, a transmembrane domain, and any of various signaling domains involved in lymphocyte activation. First generation CARs comprise a signaling domain comprising CD3-zeta alone which is requisite for a first signal (“signal 1”) of T cell activation. Second and third generation CARs additionally possess one or more co-stimulatory signaling domains (e.g., 4-1BB and/or CD28), respectively, to provide a second signal (“signal 2”).

In some embodiments of the disclosure, the inhibitor of epiregulin activity is a CAR-modified cell for example, without limitation, a CAR-modified T cell (CAR-T cell) or a CAR-modified natural killer (NK) cell (CAR-NK cell). In some embodiments, the CAR-modified cell (e.g., CAR-T cell) disrupts or kills and epiregulin-expressing cell such as, but not limited to, an epiregulin-expressing cell disclosed herein, e.g., an epiregulin-expressing dendritic cell.

Cells expressing the CARs of the present disclosure are also provided. Such cells may be immune cells, for example, but not limited to, T cells (CAR-T cells) or natural killer (NK) cells (CAR-NK cells). In some embodiments, the CAR-modified cell (e.g. CAR-T cell) disrupts or kills and epiregulin-expressing cell such as, but not limited to, an epiregulin-expressing cell disclosed herein, e.g., an epiregulin-expressing dendritic cell.

In some embodiments, CARs of the present disclosure comprise: a) an extracellular domain comprising an antigen-binding moiety that specifically binds to epiregulin; b) a transmembrane domain; and c) an cytoplasmic domain comprising one or more signaling domains. In some embodiments, the antigen-binding moiety that specifically binds to epiregulin comprises an antibody or antigen-binding fragment described herein.

CARs that may be useful in the practice of the methods disclosed herein may comprise an anti-epiregulin binding domain, a cytoplasmic domain, and a transmembrane domain. The cytoplasmic domain may comprise one or more signaling domains. The transmembrane domain is commonly located between the cytoplasmic domain and the anti-epiregulin binding domain.

The anti-epiregulin binding domain may comprise, a receptor, an antigen-binding polypeptide, or a natural ligand for a target cell antigen or receptor. The anti-epiregulin binding domain may comprise an antigen-binding polypeptide. Illustrative examples of antigen-binding polypeptides are antibodies and antibody fragments. As a non-limiting example, the antigen-binding polypeptide may be a single chain variable fragment (scFv), a rabbit antibody, a human antibody, a murine antibody, a humanized antibody, a shark antibody variable domain, a humanized version of a shark antibody variable domain, a camelized antibody variable domain, a camelid antibody variable domain, a humanized version of a camelid antibody variable domain, a single domain antibody variable domain, and a nanobody.

The transmembrane domain may be derived from CD28, CD45, CD4, DAP10, CD3-zeta, CD3-epsilon, CD5, CD7, CD9, DAP12, CD8, CD8a, CD137, CD4, CD16, CD22, CD80, CD86, CD134 (OX-40), CD33, CD37, CD40, CD64, or CD154.

The signaling domain may be derived from Fc epsilon receptor I gamma chain (FCER1G), FcR beta, CD226, CD66d, CD79A, CD79B, DAP10, DAP12 CD3-delta, CD3-epsilon, CD3-gamma, or CD3-zeta.

The cytoplasmic domain may comprise one or more co-stimulatory signaling domains. In some embodiments, the co-stimulatory signaling domain is derived from FcR beta, Fc epsilon receptor I gamma chain (FCER1G), DAP12, DAP10, CD3-delta, CD3-epsilon, CD3-gamma, CD3-zeta, CD79A, CD79B, CD226, or CD66d.

The cytoplasmic domain may comprise more than one signaling domain. For example, the cytoplasmic domain may comprise two signaling domains.

In some embodiments, the CAR further comprises one or more additional polypeptide sequences. Exemplary additional polypeptide sequences include, but are not limited to, signal sequences, epitope tags, and polypeptides that produce a detectable signal. In some embodiments, the antigen-binding domain may comprise a linker.

In some embodiments, the CAR includes a hinge domain localized between the anti-epiregulin binding domain and the transmembrane domain. The hinge domain may be an immunoglobulin hinge region.

In some embodiments, the CAR includes a leader sequence.

### Aptamers

In some embodiments of the disclosure, the inhibitor of epiregulin activity is an aptamer. Without wishing to be bound by theory, an aptamer may be a synthetic molecule, typically either polynucleotide- or peptide-based molecule, that is capable of binding specifically to another molecule. A polynucleotidal aptamer may be, without limitation, a DNA or RNA molecule, usually comprising several strands of nucleic acids, that adopt highly specific three-dimensional conformation designed to have appropriate binding affinities and specificities towards specific target molecules such as, but not limited to, peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. In some embodiments, peptide aptamers may be identified and isolated from combinatorial libraries, using methods such as the yeast two-hybrid system.

### Alternative Scaffold

Alternative scaffolds to immunoglobulin domains that exhibit similar functional characteristics, such as high-affinity and specific binding of target biomolecules, may also be used as an inhibitor of epiregulin activity of the present disclosure. Such scaffolds have been shown to result in molecules with improved characteristics, such as reduced immunogenicity or enhanced stability. Non-limiting examples of alternative scaffolds that may be useful in the practice of the methods disclosed herein include, without limitation, engineered, fibronectin-derived, 10th fibronectin type III (10Fn3) domain (e.g., monobodies. AdNectins™, or AdNexins™); engineered, ankyrin repeat motif containing polypeptide (e.g., DARPins™) lipocalin (e.g., anticalins); engineered, low-density-lipoprotein-receptor-derived. A domain (LDLR-A) (e.g., Avimers™); engineered, Protein-A-derived, 7, domain (Affibodies™) CTLD; (e.g., Tetranectin); engineered, tenascin-derived, tenascin type III domain (e.g., Centyrin™); engineered, gamma-B crystallin-derived scaffold or engineered, ubiquitin-derived scaffold (e.g., Affilins); thioredoxin (e.g., peptide aptamer); KALBITOR®; the β-sandwich (e.g., iMab); engineered, protease inhibitor-derived, Kunitz domain (e.g., EETI-II/AGRP, BPTI/LACI-D1/ITI-D2); C-type lectin-like domain scaffolds; Sac7d-derived polypeptides (e.g., Nanoffitins R or affitins); engineered, Fyn-derived, SH2 domain (e.g., Fynomers®); engineered antibody mimics; miniproteins; and any genetically manipulated counterparts of the foregoing that retains its binding functionality Skerra. Current Opin, in Biotech., 2007 18: 295-304; Binz H et al., Nat Biolechnol 23: (Worn A, Pluckthun A, J Mol Biol 305:989-1010 (2001); Xu L et al., Chem Biol 9:933-42 (2002); 1257-68 (2005); Wikman M et al., Protein Eng Des Sel 17:455-62 (2004); Byla P et al., J Biol Chem 285:12096 (2010); Zoller F et al., Molecules 16:2467-85 (2011), Hey T et al., Trends Biotechnol 23:514-522 (2005); Koide A, Koide S, Methods Mol Biol 352:95-109 (2007); Holliger P, Hudson P, Nat Biotechnol 23:1126-36 (2005); Gill D. Damle N, Curr Opin Biotech 17:653-8 (2006); each of which is incorporated by reference in its entirety for all purposes).

### Methods of Inhibiting Epiregulin

In certain aspects, the present disclosure provides a method of inhibiting an activity of epiregulin in a cell, the method comprising contacting the cell with an effective amount of an epiregulin inhibitor. In some embodiments, the activity of epiregulin is epiregulin's interaction with an ErbB receptor. In some embodiments the cell is a fibroblast or pericyte. In some embodiments, the cell is a human cell.

In some embodiments, the present disclosure provides a method of inhibiting an activity of epiregulin in a cell, the method comprising contacting the cell with an effective amount of an antibody or antigen-binding fragment of the present disclosure.

In some embodiments, the ErbB receptor is an epidermal growth factor receptor (EGFR) receptor which may also be named ErbB-1. In some embodiments, the ErbB receptor is ErbB-2 (e.g., HER2 or neu). In some embodiments, the ErbB receptor is ErbB-3 (HER3). In some embodiments, the ErbB receptor is ErbB-4 (HER4).

In some embodiments, the activity of epiregulin is reduced by at least about 5%, 10%, 20%, 30%, 40%, 50%, 60%, 70%, 80%, 90%, or more. In some embodiments, the activity of epiregulin is reduced by about 5%-20%, 10%-30%, 20%-40%, 30%-50%, 40%-60%, 50%-70%, 60%-80%, 70%-90%, 80%-95%, or more. In some embodiments, the activity of epiregulin is reduced by about 40%, 41%, 42%, 43%, 44%, 45%, 46%, 47%, 48%, 49%, 50%, 51%, 52%, 53%, 54%, 55%, 56%, 57%, 58%, 59%, 60%, or more. In some embodiments, the activity of epiregulin is reduced by about 50%.

In some embodiments, the activity of epiregulin is epiregulin's interaction with an ErbB receptor and epiregulin's interaction with the ErbB receptor is reduced by about 5%, 10%, 20%, 30%, 40%, 50%, 60%, 70%, 80%, 90%, or more. In some embodiments, the activity of epiregulin is epiregulin's interaction with an ERbB receptor and epiregulin's interaction with the ErbB receptor is reduced by about 5%-20%, 10%-30%, 20%-40%, 30%-50%, 40%-60%, 50%-70%, 60%-80%, 70%-90%, 80%-95%, or more. In some embodiments, the activity of epiregulin is epiregulin's interaction with an ErbB receptor and epiregulin's interaction with the ErbB receptor is reduced by about 40%, 41%, 42%, 43%, 44%, 45%, 46%, 47%, 48%, 49%, 50%, 51%, 52%, 53%, 54%, 55%, 56%, 57%, 58%, 59%, 60%, or more. In some embodiments, the activity of epiregulin is epiregulin's interaction with an ErbB receptor and epiregulin's interaction with the ErbB receptor is reduced by about 50%.

In some embodiments, the antibody or antigen-binding fragment of the present disclosure inhibits epiregulin-induced proliferation of the fibroblast. In some embodiments, the described antibody or antigen-binding fragment inhibits epiregulin-induced proliferation of the fibroblast by about 5%, 10%, 20%, 30%, 40%, 50%, 60%, 70%, 80%, 90%, or more. In some embodiments, the antibody or antigen-binding fragment of the present disclosure inhibits epiregulin-induced proliferation of the fibroblast by at least about 5%-20%, 10%-30%, 20%-40%, 30%-50%, 40%-60%, 50%-70%, 60%-80%, 70%-90%, 80%-95%, or more. In some embodiments, the epiregulin inhibitor inhibits epiregulin-induced proliferation of the fibroblast by about 40%, 41%, 42%, 43%, 44%, 45%, 46%, 47%, 48%, 49%, 50%, 51%, 52%, 53%, 54%, 55%, 56%, 57%, 58%, 59%, 60%, or more. In some embodiments, the epiregulin inhibitor inhibits epiregulin-induced proliferation of the fibroblast by about 50%.

### Methods of Treatment

In one aspect, the present disclosure comprises a method of reversing or preventing one or more changes in a cell associated with fibrosis, the method comprising contacting the cell with an effective amount of an epiregulin inhibitor (e.g., antibody or antigen-binding fragment) of the present disclosure.

In some embodiments, the one or more changes associated with fibrosis may be, for example, without limitation, elevated expression of fibronectin I (FN1), elevated expression of epiregulin (EREG); elevated expression of collagen type I alpha 1 chain (COL1A1); elevated expression of collagen type IV alpha 1 chain (COL4A1); elevated expression of collagen type VI alpha I chain (COL6A1); elevated expression of tenascin-C (TNC); elevated expression of fibronectin extra domain A isoform (FN^{EDA}); elevated expression of monocyte chemoattractant protein-1 (MCP-1); elevated expression of tissue inhibitor of metalloproteinase-1 (TIMP-1), or a combination thereof.

In some embodiments, the reversal of the one or more changes associated with fibrosis is a total reversal. In some embodiments, the reversal of the one or more changes associated with fibrosis is a partial reversal.

In some embodiments, the epiregulin inhibitor (e.g., antibody or antigen-binding fragment) of the present disclosure is administered in an amount sufficient to reverse the one or more changes associated with a fibrosis by at least about 10%, 20%, 30%, 40%, 50%, 60%. 70%, 80%, 90%, or 100% post administration of the epiregulin inhibitor (e.g., antibody or antigen-binding fragment). In some embodiments, the one or more changes associated with a fibrosis may be reversed by an amount greater than 100% post administration of the epiregulin inhibitor (e.g., antibody or antigen-binding fragment) of the present disclosure such that the amount of the change results in an amount that is below that which occurs in the absence of the disease or disorder, i.e., control levels. In some embodiments, the one or more changes associated with a fibrosis may be reversed by at least about 110%. 120%, 130%, 140%, 150%, 160%, 170%, 180% or 200% post administration of the epiregulin inhibitor (e.g., antibody or antigen-binding fragment).

In some embodiments, the epiregulin inhibitor (e.g., antibody or antigen-binding fragment) of the present disclosure is administered in an amount sufficient to reverse elevated expression of fibronectin I (FN1) by at least about 10%, 20%, 30%, 40%, 50%, 60%, 70%, 80%, 90%, or 100% post administration of the epiregulin inhibitor (e.g., antibody or antigen-binding fragment). In some embodiments, the epiregulin inhibitor (e.g., antibody or antigen-binding fragment) of the present disclosure is administered in an amount sufficient to reverse elevated expression of fibronectin I (FN1) by at least about 110%. 120%, 130%, 140%, 150%, 160%, 170%, 180%, 200%, 300%, 400% or 500% post administration of the epiregulin inhibitor (e.g., antibody or antigen-binding fragment).

In some embodiments, the epiregulin inhibitor (e.g., antibody or antigen-binding fragment) of the present disclosure is administered in an amount sufficient to reverse elevated expression of EREG by at least about 10%, 20%, 30%, 40%, 50%, 60%, 70%, 80%, 90%, or 100% post administration of the epiregulin inhibitor (e.g., antibody or antigen-binding fragment). In some embodiments, the epiregulin inhibitor (e.g., antibody or antigen-binding fragment) of the present disclosure is administered in an amount sufficient to reverse elevated expression of EREG by at least about 110%. 120%. 130%. 140%, 150%, 160%, 170%. 180%, 200%, 300%, 400% or 500% post administration of the epiregulin inhibitor (e.g, antibody or antigen-binding fragment).

In some embodiments, the epiregulin inhibitor (e.g., antibody or antigen-binding fragment) of the present disclosure is administered in an amount sufficient to reverse elevated expression of COL1A1 by at least about 10%, 20%, 30%, 40%, 50%, 60%, 70%, 80%, 90%, or 100% post administration of the epiregulin inhibitor (e.g., antibody or antigen-binding fragment). In some embodiments, the epiregulin inhibitor (e.g., antibody or antigen-binding fragment) of the present disclosure is administered in an amount sufficient to reverse elevated expression of COL1A1 by at least about 110%, 120%, 130%, 140%, 150%. 160%, 170%, 180%, 200%, 300%, 400% or 500% post administration of the epiregulin inhibitor (e.g., antibody or antigen-binding fragment).

In some embodiments, the epiregulin inhibitor (e.g., antibody or antigen-binding fragment) of the present disclosure is administered in an amount sufficient to reverse elevated expression of COL4A1 by at least about 10%, 20%, 30%, 40%, 50%, 60%, 70%, 80%, 90%, or 100% post administration of the epiregulin inhibitor (e.g., antibody or antigen-binding fragment). In some embodiments, the epiregulin inhibitor (e.g., antibody or antigen-binding fragment) of the present disclosure is administered in an amount sufficient to reverse elevated expression of COL4A1 by at least about 110%, 120%, 130%, 140%, 150%. 160%, 170%, 180%, 200%, 300%, 400% or 500% post administration of the epiregulin inhibitor (e.g., antibody or antigen-binding fragment).

In some embodiments, the epiregulin inhibitor (e.g., antibody or antigen-binding fragment) of the present disclosure is administered in an amount sufficient to reverse elevated expression of COL6A1 by at least about 10%, 20%, 30%, 40%, 50%, 60%, 70%, 80%, 90%, or 100% post administration of the epiregulin inhibitor (e.g., antibody or antigen-binding fragment). In some embodiments, the epiregulin inhibitor (e.g., antibody or antigen-binding fragment) of the present disclosure is administered in an amount sufficient to reverse elevated expression of COL6A1 by at least about 110%, 120%, 130%, 140%, 150%. 160%, 170%, 180%, 200%, 300%, 400% or 500% post administration of the epiregulin inhibitor (e.g., antibody or antigen-binding fragment).

In some embodiments, the epiregulin inhibitor (e.g., antibody or antigen-binding fragment) of the present disclosure is administered in an amount sufficient to reverse elevated expression of TNC by at least about 10%, 20%, 30%, 40%, 50%, 60%, 70%, 80%, 90%, or 100% post administration of the epiregulin inhibitor (e.g., antibody or antigen-binding fragment). In some embodiments, the epiregulin inhibitor (e.g., antibody or antigen-binding fragment) of the present disclosure is administered in an amount sufficient to reverse elevated expression of TNC by at least about 110%, 120%, 130%, 140%, 150%, 160%, 170%, 180%, 200%, 300%, 400% or 500% post administration of the epiregulin inhibitor (e.g., antibody or antigen-binding fragment).

In some embodiments, the epiregulin inhibitor (e.g., antibody or antigen-binding fragment) is administered in an amount sufficient to reverse elevated expression of FN^{EDA} by at least about 10%, 20%, 30%, 40%, 50%, 60%, 70%, 80%, 90%, or 100% post administration of the antibody or antigen-binding fragment. In some embodiments, the epiregulin inhibitor (e.g., antibody or antigen-binding fragment) of the present disclosure is administered in an amount sufficient to reverse elevated expression of FN^{EDA} by at least about 110%, 120%, 130%, 140%, 150%, 160%, 180%, 200%, 300%, 400% or 500% post administration of the epiregulin inhibitor (e.g., antibody or antigen-binding fragment).

In some embodiments, the epiregulin inhibitor (e.g., antibody or antigen-binding fragment) of the present disclosure is administered in an amount sufficient to reverse elevated expression of MCP-1 by at least about 10%, 20%, 30%, 40%, 50%, 60%, 70%, 80%, 90%, or 100% post administration of the epiregulin inhibitor (e.g., antibody or antigen-binding fragment). In some embodiments, the epiregulin inhibitor (e.g., antibody or antigen-binding fragment) of the present disclosure is in an amount sufficient to reverse elevated expression of MCP-1 by at least about 110%, 120%, 130%, 140%, 150%, 160%, 170%, 180% or 200% post administration of the epiregulin inhibitor (e.g., antibody or antigen-binding fragment).

In some embodiments, the epiregulin inhibitor (e.g., antibody or antigen-binding fragment) of the present disclosure is administered in an amount sufficient to reverse elevated expression of TIMP-1 by at least about 10%, 20%, 30%, 40%, 50%, 60%, 70%, 80%, 90%, or 100% post administration of the epiregulin inhibitor (e.g., antibody or antigen-binding fragment). In some embodiments, the epiregulin inhibitor (e.g., antibody or antigen-binding fragment) of the present disclosure is administered in an amount sufficient to reverse elevated expression of TIMP-1 by at least about 110%, 120%, 130%, 140%, 150%, 160%, 170%, 180% or 200% post administration of the epiregulin inhibitor (e.g., antibody or antigen-binding fragment).

In some embodiments, the cell is a fibroblast or pericyte.

In some embodiments, the cell is a human cell.

In one aspect, the present disclosure comprises a method of treating or preventing a fibrotic disease and/or disorder in a subject (e.g., a human subject) in need thereof, the method comprising administering a therapeutically effective amount of a described epiregulin inhibitor (e.g., antibody or antigen-binding fragment) to the subject. In some embodiments, the administration of the described epiregulin inhibitor (e.g., antibody or antigen-binding fragment) results in the reversal of the fibrotic disease and/or disorder. In some embodiments, the reversal of the fibrotic disease and/or disorder is a total reversal of the fibrotic disease and/or disorder. In some embodiments, the reversal is a partial reversal of the fibrotic disease and/or disorder.

In some embodiments, the described epiregulin inhibitor (e.g., antibody or antigen-binding fragment) is administered in an amount sufficient to reverse the fibrotic disease and/or disorder by at least about 10%, 20%, 30%, 40%, 50%, 60%, 70%, 80%, 90%, or 100% post administration of the described antibody or antigen-binding fragment.

### Fibrotic Diseases and Disorders

The disclosure may be practiced in any subject diagnosed with, or at risk of developing, fibrosis. Fibrosis is associated with many diseases and disorders. The subject may be diagnosed with, or at risk for developing, for example, interstitial lung disease including idiopathic pulmonary fibrosis, scleroderma (or sclerosis) including both systemic scleroderma (affecting internal organs) and localized scleroderma (skin only) which may also be referred to as morphea, gastrointestinal fibrosis, cardiac fibrosis, radiation-induced pulmonary fibrosis, bleomycin lung, sarcoidosis, silicosis, familial pulmonary fibrosis, an autoimmune disease or any disorder wherein one or more fibroproliferative matrix molecule deposition, enhanced pathological collagen accumulation, apoptosis and alveolar septal rupture with honeycombing occurs. The subject may be identified as having fibrosis or being at risk for developing fibrosis because of exposure to asbestos, ground stone and metal dust, or because of the administration of a medication, such as bleomycin, busulfon, pheytoin, and nitro furantoin, which are risk factors for developing fibrosis. Preferably, the subject is a mammal and more preferably, a human.

Other illustrative examples of fibrotic diseases include, without limitation, scleromyxedema, nephrogenic systemic fibrosis (also called nephrogenic fibrosing dermopathy), and long COVID syndrome.

It is also contemplated that the compositions and methods of the disclosure may be used in the treatment of organ fibrosis secondary to allogenic organ transplant, e.g., graft transplant fibrosis or graft-versus-host disease including, without limitation, chronic graft-versus-host disease, sclerotic graft-vs-host disease, and sclerotic graft-vs-host disease skin fibrosis. Non-limiting examples include renal transplant fibrosis, heart transplant fibrosis, liver transplant fibrosis, etc. In some embodiments, the compositions and methods of the disclosure may be used to treat, e.g., Bronchiolitis obliterans syndrome which can be due to lung transplant rejection.

In some embodiments, the method of treating or preventing a fibrotic disease or disorder disclosed herein may comprise treatment or prevention of a fibrotic disease or disorder such as, but not limited to, scleroderma, interstitial lung disease, gastrointestinal fibrosis, cardiac fibrosis, skin fibrosis (e.g., systemic sclerosis skin fibrosis, or sclerotic graft-vs-host disease skin fibrosis), scleromyxedema, nephrogenic systemic fibrosis (nephrogenic fibrosing dermopathy), chronic graft-versus-host disease, sclerotic graft-vs-host disease, Bronchiolitis obliterans syndrome, keloid scar, a fibrosis (e.g., lung fibrosis) associated with or due to COVID-19 (e.g, SARS-COV-2 infections) including, for example, long COVID syndrome, or a combination thereof. In some embodiments, the scleroderma is system systemic scleroderma, in some embodiments, the scleroderma is localized scleroderma (morphea). In some embodiments, the interstitial lung disease is idiopathic pulmonary fibrosis.

In some embodiments, the fibrosis is pulmonary fibrosis. As used herein, the term “pulmonary fibrosis” or “fibrotic lung disease” or “fibroid lung disease” or “scarring of the lung” refers to a group of diseases characterized by the formation or development of excess fibrous connective tissue (fibrosis) in the lungs. Symptoms of pulmonary fibrosis are mainly: shortness of breath, particularly with exertion; chronic dry, hacking coughing; fatigue and weakness; chest discomfort; and loss of appetite and rapid weight loss. Pulmonary fibrosis may be a secondary effect of other diseases, most of them being classified as interstitial lung diseases, such as autoimmune disorders, viral infections or other microscopic injuries to the lung. Pulmonary fibrosis can also appear without any known cause (“idiopathic”). Idiopathic pulmonary fibrosis is a diagnosis of exclusion of a characteristic set of histologic/pathologic features known as usual interstitial pneumonia (UIP).

Diseases and conditions that may cause pulmonary fibrosis as a secondary effect include: inhalation of environmental and occupational pollutants (asbestosis, silicosis and gas exposure); hypersensitivity pneumonitis, most often resulting from inhaling dust contaminated with bacterial, fungal, or animal products; cigarette smoking; connective tissue diseases such as rheumatoid arthritis. SLE; scleroderma, sarcoidosis and Wegener's granulomatosis; infections; medications such as amiodarone, bleomycin (pingyangmycin), busulfan, methotrexate, apomorphine and nitrofurantoin; and radiation therapy to the chest.

In some embodiments, the compositions and methods of the disclosure may be used in the treatment or prevention of idiopathic pulmonary fibrosis (IPF). IPF is a devastating chronic lung disease with yet unknown etiology. IPF leads to death in 3.5-4 years from initial diagnosis in more than 50% of the patients, irrespective of treatment (Travis, et al., 2013. Am. J. Resp. Crit. Care Med. 188:733-748). Despite extensive research efforts, its pathogenesis is still elusive and controversial (Selman, et al., 2001, Ann. Int. Med. 134:136-151; Selman, et al., 2008, PLOS Med. 5: e62).

With a gradually increasing worldwide incidence and no proven therapies other than lung transplantations, IPF treatment represents a major challenge for both pharmaceutical industries and chest physicians. To date, all available treatment agents have been delivered systemically, either orally or subcutaneously. In addition to their limited therapeutic efficacy, use of the majority of these agents has been associated with side effects, ranging from major side effects (such as immune suppression and subsequent infections, acute exacerbations of disease and excessive bleeding) to minor side effects (including gastrointestinal complications, such as diarrhea and nausea) that significantly affect patient quality of life. So far, none of the agents tried, had any significant effect on patient survival.

In some embodiments, the compositions and methods of the disclosure may be used in the treatment or prevention of scleroderma. Scleroderma is a chronic connective tissue disease generally classified as one of the autoimmune rheumatic diseases. Patients with scleroderma can have specific antibodies (ANA, anticentromere, or antitopoisomerase) in their blood that suggest autoimmunity. Symptoms can generally include thickened skin that can involve scarring, blood vessel problems, varying degrees of inflammation and pain, and is associated with an overactive immune system.

Scleroderma can be classified in terms of the degree and location of the skin and organ involvement. Accordingly, scleroderma has been categorized into two major groups, localized scleroderma and systemic sclerosis, which can be further subdivided into either diffuse or limited forms based on the location and extent of skin involvement. Localized scleroderma skin changes are in isolated areas, either as morphea patches or linear scleroderma. Morphea is scleroderma that is localized to a patchy area of the skin that becomes hardened and slightly pigmented. Sometimes morphea can cause multiple lesions in the skin. Morphea is not associated with disease elsewhere within the body, only in the involved skin areas. Linear scleroderma is scleroderma that is localized usually to a lower extremity, frequently presenting as a strip of hardening skin down the leg of a child. Linear scleroderma in children can stunt bone growth of the affected limb. Sometimes linear scleroderma is associated with a “satellite” area of a patch of localized scleroderma skin, such as on the abdomen.

The widespread type of scleroderma involves internal organs in addition to the skin. This type, called systemic sclerosis, is subcategorized by the extent of skin involvement as either diffuse or limited. The diffuse form of scleroderma (diffuse systemic sclerosis) involves symmetric thickening of skin of the extremities, face, and trunk (chest, back, abdomen, or flanks) that can rapidly progress to hardening after an early inflammatory phase. Organ disease can occur early on and be serious and significantly decrease life expectancy. Organs affected include the esophagus, bowels, and scarring (fibrosis) of the lungs, heart, and kidneys. High blood pressure can be troublesome and can lead to kidney failure (renal crisis).

The limited form of scleroderma tends to have far less skin involvement with skin thickening confined to the skin of the fingers, hands, and face. The skin changes and other features of disease tend to occur more slowly than in the diffuse form. Because characteristic clinical features can occur in patients with the limited form of scleroderma, this form has taken another name that is composed of the first initials of the common components. Thus, this form is also called the “CREST” variant (subset thereof, e.g., CRST, REST, or ST) of scleroderma. CREST syndrome represents the following features: Calcinosis (the formation of tiny deposits of calcium in the skin), Raynaud's phenomenon (the spasm of the tiny arterial vessels supplying blood to the fingers, toes, nose, tongue, or ears), Esophagus disease (characterized by poorly functioning muscle of the lower two-thirds of the esophagus). Sclerodactyly (localized thickening and tightness of the skin of the fingers or toes), and Telangiectasias (tiny red areas, frequently on the face, hands, and in the mouth behind the lips).

Some subjects have scleroderma and one or more other connective tissue diseases, such as rheumatoid arthritis, systemic lupus erythematosus, and polymyositis Features of scleroderma along with features of polymyositis, systemic lupus erythematosus, and certain abnormal blood tests, can lead to a diagnosis of mixed connective tissue disease (MCTD).

### Combination Therapy

In certain embodiments, the described epiregulin inhibitors (e.g., antibodies or antigen-binding fragments) of the disclosure are useful in the methods of the disclosure in combination with at least one additional therapeutic agent useful for treating or preventing fibrotic disease and/or disorder. This additional therapeutic agent may comprise therapeutic agents identified herein or another therapeutic agent, e.g., commercially available therapeutic agent, known to treat, prevent or reduce the symptoms of fibrotic lung disease.

Non-limiting examples of additional therapeutic agents contemplated for use in accordance with the disclosure include mycophenolate mofetil, nintedanib, tocilizumab, pirfenidone, rituximab, prednisone or another corticosteroid medication, methotrexate, UVA or UVB phototherapy, extracorporeal photopheresis, stem cell transplant, and cyclophosphamide.

A synergistic effect may be calculated, for example, using suitable methods such as, for example, the Sigmoid-Eₘₐₓ equation (Holford & Scheiner, 19981, Clin. Pharmacokinet. 6:429-453), the equation of Loewe additivity (Loewe & Muischnek, 1926. Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Dosing, Formulation, Administration

The dose of the described epiregulin inhibitor (e.g., antibody or antigen-binding fragment) administered to a subject (such as a human) may vary with the particular composition, the method of administration, and the particular kind and stage of disease or disorder (such as a fibrotic disease or disorder) being treated. The amount should be sufficient to produce a desirable response, such as a therapeutic response against the disease or disorder (such as a fibrotic disease or disorder). In some embodiments, the amount of the composition (e.g., the described epiregulin inhibitor (e.g., antibody or antigen-binding fragment)) is a therapeutically effective amount.

In some embodiments, the amount of the composition is an amount sufficient to promote normalization and/or improvement of dermal thickness. In some embodiments, the amount of the composition is an amount sufficient to promote normalization and/or improvement of lung fibrosis. In some embodiments, the amount of the composition is an amount sufficient to promote normalization and/or improvement of alveolar septae. In some embodiments, the amount of the composition is an amount sufficient to reduce or prevent fibrotic masses.

In some embodiments, the amount of the composition is an amount sufficient to reverse or prevent one or more changes in a cell associated with fibrosis. As a nonlimiting example, the changes associated with fibrosis may include elevated FN1; elevated expression of EREG; elevated expression of COL1A1; elevated expression of COL4A1; elevated expression of COL6A1; elevated expression of TNC; elevated expression of FN^{EDA}, elevated expression of MCP-1; elevated expression of TIMP-1, or a combination thereof.

In some embodiments, the amount of the composition is an amount sufficient to produce a decrease in FN1 by at least about 10%, 20%, 30%, 40%, 50%, 60%, 70%, 80%, 90%, or 100% post administration of the composition. In some embodiments, the composition is in an amount sufficient to reverse elevated expression of fibronectin I (FN1) by at least about 110%, 120%, 130%, 140%, 150%, 160%, 170%, 180%, 200%, 300%, 400% or 500% post administration of the composition.

In some embodiments, the amount of the composition is an amount sufficient to produce a decrease in EREG by at least about 10%, 20%, 30%, 40%, 50%, 60%, 70%, 80%. 90%, or 100% post administration of the composition. In some embodiments, the composition is in an amount sufficient to reverse elevated expression of EREG by at least about 110%, 120%, 130%, 140%, 150%, 160%, 170%, 180%, 200%, 300%, 400% or 500% post administration of the composition.

In some embodiments, the amount of the composition is an amount sufficient to produce a decrease in COL1A1 by at least about 10%, 20%, 30%, 40%, 50%, 60%, 70%, 80%, 90%, or 100% post administration of the composition. In some embodiments, the composition is in an amount sufficient to reverse elevated expression of COL1A1 by at least about 110%, 120%, 130%, 140%, 150%, 160%, 170%, 180%, 200%, 300%, 400% or 500% post administration of the composition.

In some embodiments, the amount of the composition is an amount sufficient to produce a decrease in COL4A1 by at least about 10%, 20%, 30%, 40%, 50%, 60%, 70%, 80%, 90%, or 100% post administration of the composition. In some embodiments, the composition is in an amount sufficient to reverse elevated expression of COL4A1 by at least about 110%, 120%, 130%, 140%, 150%, 160%, 170%, 180%, 200%, 300%, 400% or 500% post administration of the composition.

In some embodiments, the amount of the composition is an amount sufficient to produce a decrease in COL6A1 by at least about 10%, 20%, 30%, 40%, 50%, 60%, 70%, 80%, 90%, or 100% post administration of the composition. In some embodiments, the composition is in an amount sufficient to reverse elevated expression of COL6A1 by at least about 110%, 120%, 130%, 140%, 150%, 160%, 170%, 180%, 200%, 300%, 400% or 500% post administration of the composition.

In some embodiments, the amount of the composition is an amount sufficient to produce a decrease in TNC by at least about 10%, 20%, 30%, 40%, 50%, 60%, 70%, 80%, 90%, or 100% post administration of the composition. In some embodiments, the composition is in an amount sufficient to reverse elevated expression of TNC by at least about 110%, 120%, 130%, 140%, 150%, 160%, 170%, 180%, 200%, 300%, 400% or 500% post administration of the composition.

In some embodiments, the amount of the composition is an amount sufficient to produce a decrease in FN^{EDA} by at least about 10%, 20%, 30%, 40%, 50%, 60%, 70%, 80%, 90%, or 100% post administration of the composition. In some embodiments, the composition is in an amount sufficient to reverse elevated expression of FN^{EDA} by at least about 110%, 120%, 130%, 140%, 150%, 160%, 170%, 180%, 200%, 300%, 400% or 500% post administration of the composition.

In some embodiments, the amount of the composition is an amount sufficient to produce a decrease in MCP-1 by at least about 10%, 20%, 30%, 40%, 50%, 60%, 70%, 80%, 90%, or 100% post administration of the composition. In some embodiments, the composition is in an amount sufficient to reverse elevated expression of MCP-1 by at least about 110%, 120%, 130%, 140%, 150%, 160%, 170%, 180%, 200%, 300%, 400% or 500% post administration of the composition.

In some embodiments, the amount of the composition is an amount sufficient to produce a decrease in TIMP-1 by at least about 10%, 20%, 30%, 40%, 50%, 60%, 70%, 80%, 90%, or 100% post administration of the composition.

In addition to hydroxyproline assay of collagen, assays to measure any of the above changes in, e.g., FN1 and TNC expression and/or quantity include, include, but are not limited to, quantitative polymerase chain reaction (qPCR), microarray, RNA sequencing (RNA-Seq), single-cell RNA-Seq (scRNA-Seq), enzyme-linked immunoassay (ELISA), mass spectrometry, and Western blot.

Any of the described epiregulin inhibitors (e.g., antibodies or antigen-binding fragments) described herein can be present in a composition such as a formulation that includes other agents, excipients, or stabilizers.

In some embodiments, the composition further comprises a target agent or a carrier that promotes the delivery of the described epiregulin inhibitor (e.g., antibody or antigen-binding fragment) to a fibrotic tissue or a tissue associated with a fibrotic disease and/or disorder. Exemplary carriers include liposomes, micelles, nanodisperse albumin and its modifications, polymer nanoparticles, dendrimers, inorganic nanoparticles of different compositions.

In some embodiments, the composition is suitable for administration to a human. In some embodiments, the composition is suitable for administration to a mammal such as, in the veterinary context, domestic pets and agricultural animals.

In some embodiments, the composition is administered to a subject (e.g, a human subject), after the onset of the fibrotic disease and/or disorder. In some embodiments, the composition is administered 1 day, 2 days, 3 days, 4 days, 5 days, 6 days, or 7 days or more days after the onset of the fibrotic disorder. In some embodiments, the composition is administered 1 week, 2 weeks, 3 weeks, 4 weeks, 5 weeks, 6 weeks, 7 weeks, 8 weeks, 9 weeks, 10 weeks, 20 week, 30 weeks, 40 weeks, 50 weeks, or more after the onset of the fibrotic disease or disorder. In some embodiments, the composition is administered 1 week, 2 years, 3 years, 4 years, 5 years, 6 years. 7 years, 8 years, 9 years, 10 years, 20 week, 30 years, 40 years, 50 years, or more after the onset of the fibrotic disease or disorder.

In some embodiments, the composition is administered to a subject (e.g, a human subject), prior to the onset of the fibrotic disease and/or disorder. In some embodiments, the composition is administered 1 day, 2 days, 3 days, 4 days, 5 days, 6 days, or 7 days or more prior to the onset of the fibrotic disorder. In some embodiments, the composition is administered 1 week, 2 weeks, 3 weeks, 4 weeks, 5 weeks, 6 weeks, 7 weeks, 8 weeks, 9 weeks, 10 weeks, 20 week, 30 weeks. 40 weeks, 50 weeks, or more prior to the onset of the fibrotic disease or disorder. In some embodiments, the composition is administered 1 week, 2 years, 3 years, 4 years, 5 years, 6 years. 7 years, 8 years, 9 years, 10 years, 20 week, 30 years, 40 years, 50 years, or more prior to the onset of the fibrotic disease or disorder.

There are a wide variety of suitable formulations of the composition comprising a described epiregulin inhibitor (e.g., antibody or antigen-binding fragment) disclosed herein. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl and propylhydroxy benzoates, talc, magnesium stearate, and mineral oil. In some embodiments, the composition comprising the described epiregulin inhibitor (e.g., antibody or antigen-binding fragment) with a carrier as discussed herein is present in a dry formulation (such as lyophilized composition). The formulations can additionally include lubricating agents, wetting agents, emulsifying and suspending agents, preserving agents, sweetening agents or flavoring agents.

In some embodiments, the compositions are formulated to be administered by any route which results in a therapeutically effective outcome. These include but are not limited to administered intravenously, intraarterially, intraperitoneally, intravesicularly, subcutaneously, intrathecally, intrapulmonarily, intramuscularly, intratracheally, intraocularly, transdermally, orally, or by inhalation.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is advisable to include isotonic agents, for example, sugars, sodium chloride, or polyalcohols such as mannitol and sorbitol, in the composition.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder contemplated in the disclosure.

Formulations may be employed in admixtures with conventional excipients, i.e., pharmaceutically acceptable organic or inorganic carrier substances suitable for any suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., analgesic agents.

Suitable compositions and dosage forms include, for example, dispersions, suspensions, solutions, syrups, granules, beads, powders, pellets, liquid sprays for nasal or oral administration, dry powder or aerosolized formulations for inhalation, and the like. Powdered and granular formulations of a pharmaceutical preparation of the disclosure may be prepared using known methods. Such formulations may be administered directly to a subject, used, for example, to form a material that is suitable to administration to a subject. Each of these formulations may further comprise one or more of dispersing or wetting agent, a suspending agent, and a preservative. Additional excipients, such as fillers and sweetening, flavoring, or coloring agents, may also be included in these formulations.

Pharmaceutical compositions of the disclosure may also be formulated to provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. It should be understood that the formulations and compositions that would be useful in the present disclosure are not limited to the particular formulations and compositions that are described herein.

### Kits

Kits provided herein include one or more containers comprising the described epiregulin inhibitor or a pharmaceutical composition comprising the described epiregulin inhibitor described herein and/or other agent(s), and in some embodiments, further comprise instructions for use in accordance with any of the methods described herein. The kit may further comprise a description of selection of subject suitable for treatment. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

In some embodiments, the kit comprises a) a composition comprising an anti-epiregulin antibody and/or antigen-binding fragment described herein, or a pharmaceutically acceptable salt thereof and a pharmaceutically acceptable carrier; and optionally b) instructions for administering the described antibody or antigen-binding fragment for treatment of a disease or disorder.

In some embodiments, the kit comprises a) a composition comprising an epiregulin inhibitor comprising a decoy receptor such as, but not limited to, a soluble ErbB receptor, e.g., a soluble EGFR, or a pharmaceutically acceptable salt thereof and a pharmaceutically acceptable carrier; and optionally b) instructions for administering the epiregulin inhibitor for treatment of a disease or disorder.

In some embodiments, the kit comprises a) a composition comprising CAR-modified cells (e.g., CAR-T cells) described herein, or a pharmaceutically acceptable salt thereof and a pharmaceutically acceptable carrier; and optionally b) instructions for administering CAR-modified cells (e.g., CAR-T cells) for treatment of a disease or disorder.

In some embodiments, the kit comprises a) a composition comprising an epiregulin inhibitor comprising an aptamer, or a pharmaceutically acceptable salt thereof and a pharmaceutically acceptable carrier; and optionally b) instructions for administering the epiregulin inhibitor for treatment of a disease or disorder.

In some embodiments, the kit comprises a) a composition comprising an epiregulin inhibitor comprising an alternative scaffold, or a pharmaceutically acceptable salt thereof and a pharmaceutically acceptable carrier; and optionally b) instructions for administering the epiregulin inhibitor for treatment of a disease or disorder.

The kits of the disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

In some embodiments, the kits comprise one or more components that facilitate delivery of the described epiregulin inhibitor (e.g., antibody or antigen-binding fragment), or a composition comprising the agent, and/or additional therapeutic agents to the subject. In some embodiments, the kit comprises, e.g., syringes and needles suitable for delivery of cells to the subject, and the like. In such embodiments, the described epiregulin inhibitor (e.g., antibody or antigen-binding fragment), or a composition comprising the agent may be contained in the kit in a bag, or in one or more vials. In some embodiments, the kit comprises components that facilitate intravenous or intra-arterial delivery of the described epiregulin inhibitor (e.g., antibody or antigen-binding fragment), or a composition comprising the agent to the subject. In some embodiments, the described epiregulin inhibitor (e.g., antibody or antigen-binding fragment), or a composition comprising the agent may be contained, e.g., within a bottle or bag (for example, a blood bag or similar bag able to contain up to about 1.5 L solution comprising the cells), and the kit further comprises tubing and needles suitable for the delivery of the described epiregulin inhibitor (e.g., antibody or antigen-binding fragment), or a composition comprising the agent to the subject.

The instructions relating to the use of the compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the described epiregulin inhibitor (e.g., antibody or antigen-binding fragment) as disclosed herein to provide effective treatment of a subject for an extended period, such as any of 1 day, 2 days, 3 days, 4 days, 5 days, 6 days. 7 days, 8 days, 9 days, 10 days, 11 days. 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the pharmaceutical compositions and instructions for use and packaged in quantities sufficient for storage and use in pharmacies, for example, hospital pharmacies and compounding pharmacies.

### EXAMPLES

The following examples are provided to further describe some of the embodiments disclosed herein. The examples are intended to illustrate, not to limit, the disclosed embodiments.

### Example 1. Generation of Human Epiregulin Neutralizing Antibody

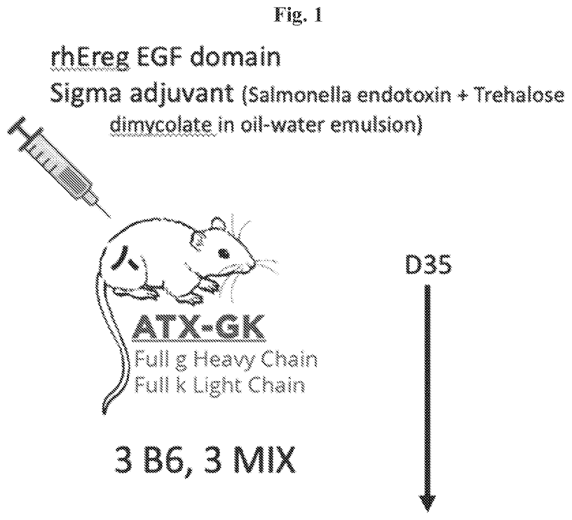

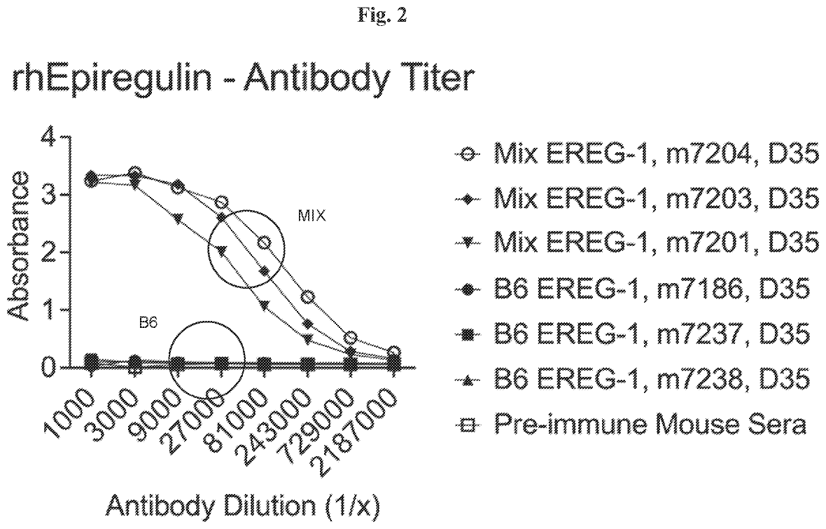

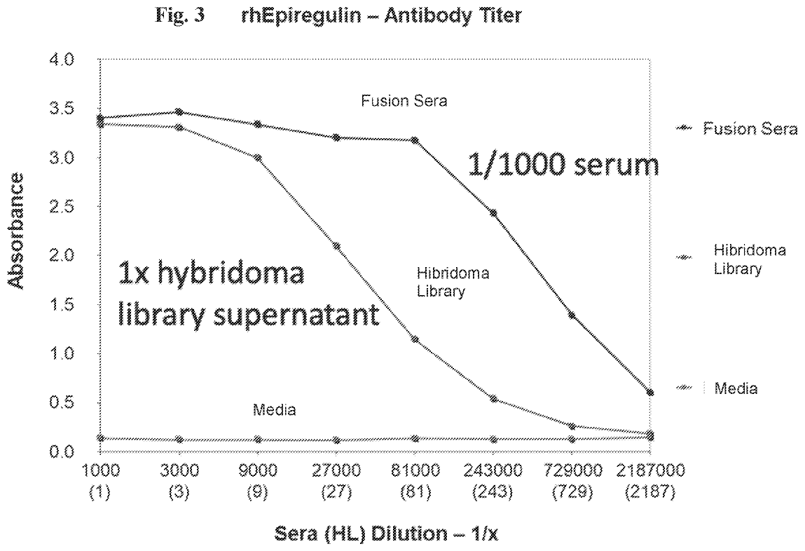

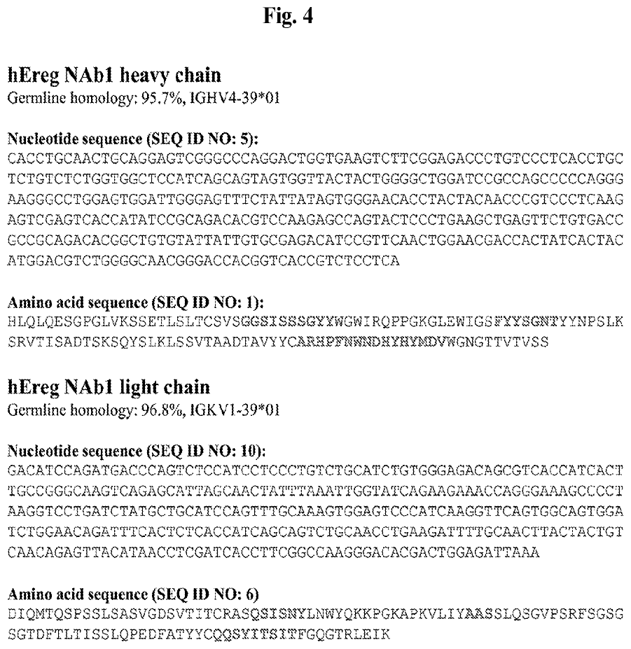

The present work sought to generate humanized epiregulin neutralizing antibodies for therapeutic treatment of scleroderma (systemic sclerosis/SSc) associated fibrosis of the skin and lung, as well as other fibrotic diseases such as idiopathic pulmonary fibrosis, chronic graft-vs-host disease, and pulmonary fibrosis complicating COVID19 infection. To accomplish the goals proposed of the present work, projects were set up to generate humanized epiregulin antibodies using Alloy mice, which can generate fully human antibody variable regions. Alloy mice (ATX GK-BL6 and ATX GK MIX) were used. To use as immunogen, a bulk order of recombinant human epiregulin containing the epidermal growth factor (EGF) domain was purchased from R&D Biosciences (Cat #1195-EP). Three of each Alloy mouse strain were immunized with recombinant epiregulin protein (diagramed in FIG. 1). Day 35 after immunization, epiregulin antibody titers were measured. Mixed, but not the B6, background of Alloy mice produced positive epiregulin titers (FIG. 2). B cells from the spleens of the mixed mice were pooled and used to generate a hybridoma library. From the hybridoma library, cells were singly plated yielding 5 wells whose supernatant contained epiregulin-binding antibody by ELISA (FIG. 3 and Table 1). The variable regions of the heavy and light chains of these clones were sequenced, from which 2 unique sequences were found. As discussed below, the unique clone that represented wells M3, M4 and M5 was found to be neutralizing against human epiregulin protein. The sequence of the antibody generated by M3-5 is shown in FIG. 4, and the antibody clone is referred to herein as humanized epiregulin neutralizing antibody 1 (hEreg NAb1).

TABLE 1
rhEpiregulin - Antibody Titer

| T.C./ELISA Well | O.D. | |
|---|---|---|
| 2B12 | 0.32 | |
| 5H07 | 0.60 | M1 |
| 5H08 | 0.47 | |
| 6A11 | 0.32 | |
| 6B09 | 0.66 | M2 |
| 6F09 | 0.27 | |
| 6F11 | 0.34 | |
| 12B03 | 3.29 | M3 |
| 12E08 | 3.43 | M4 |
| 20D08 | 0.22 | |
| 25H09 | 0.27 | |
| 25H10 | 0.31 | |
| 30F01 | 2.57 | M5 |

### Example 2, hEreg NAb1 Neutralizes Recombinant and Full-Length Epiregulin Protein

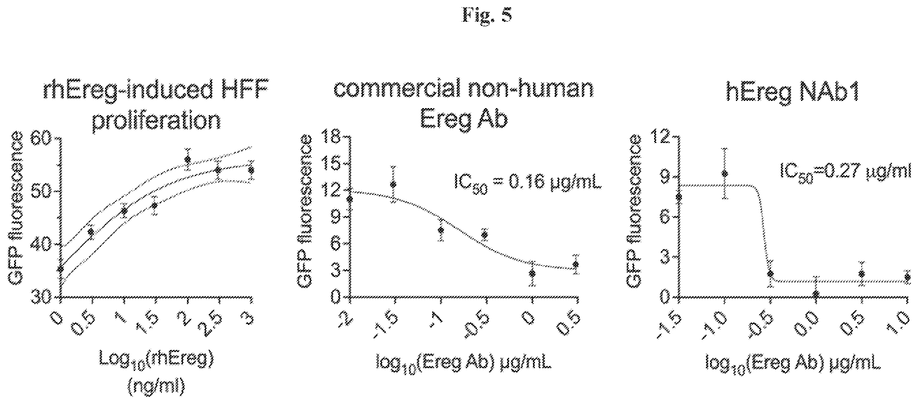

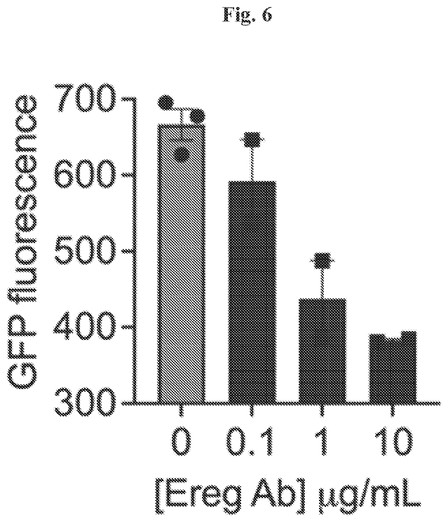

To gauge their ability to inhibit epiregulin, whether the two unique antibodies produced by the hybridoma clones could inhibit recombinant epiregulin protein in vitro was tested. Characteristic of other EGFR ligands, recombinant human epiregulin (same as used for Alloy mouse immunization) induced proliferation of cultured human foreskin fibroblasts (HFF, ATCC #SCRC-1041) (FIG. 5, left panel). Maximal proliferation occurred at 100 ng/ml, so this concentration was used in subsequent inhibition experiments. The ability of hEreg NAb1 and a commercially available rat anti-human/mouse neutralizing epiregulin antibody (R&D Systems MAB1068) to inhibit epiregulin-induced HFF proliferation was then compared. Like the commercial neutralizing antibody, hEreg NAb1 inhibited HFF proliferation in a concentration-dependent manner (FIG. 5, middle panel and right panel). The IC₅₀s of the commercial antibody and hEreg NAb1 were similar, with 0.16 μg/ml for the commercial antibody and between 0.1-0.3 μg/ml for the humanized antibody. Cultured HFF expressed endogenous full-length epiregulin, but little to no other EGFR ligands. If HFF depend on the expression of epiregulin for growth and survival, inhibition of the endogenously produced protein should reduce their growth. HFF were therefore incubated with increasing concentrations of hEreg NAb1 and decreased growth was observed, which was attributed to inhibition of the full-length epiregulin protein by hEreg NAb1 (FIG. 6). Thus, hEreg NAb1 inhibits both the recombinant and full-length human epiregulin proteins.

### Example 3, hEreg NAb1 does not Cross-React with Mouse Epiregulin

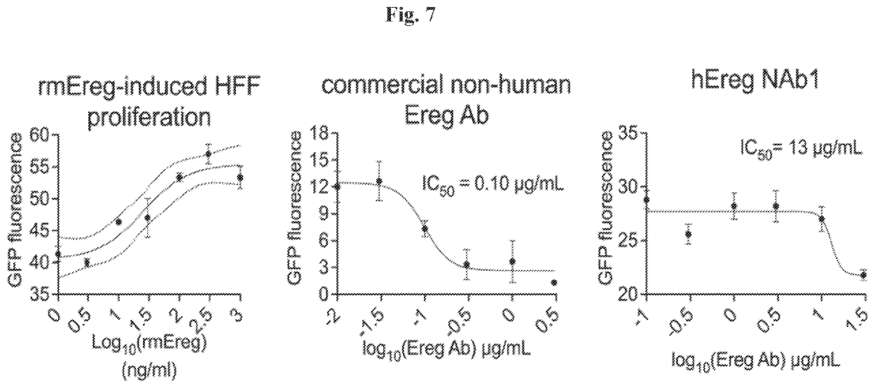

In order to validate the humanized antibody in mouse models of fibrosis, it was necessary to establish whether it cross-reacted with the mouse epiregulin homologue. To setup the assay, if recombinant mouse epiregulin could induce HFF proliferation was tested first, which it did similar to the human protein (FIG. 7, left panel). The same commercially available epiregulin antibody used above was also inhibited HFF proliferation by mouse epiregulin (FIG. 7, middle panel). In contrast, hEreg NAb1 required >100-fold higher concentration to inhibit HFF proliferation by mouse epiregulin protein (FIG. 7, right panel). These results show that hEreg NAb1 has little cross-reactivity for the mouse epiregulin protein. These findings are likely due to raising the humanized antibody in mice, which would negatively select out B cells that produce a self-reactive antibody.

### Example 4, hEreg NAb1 Shows No Cross-Reactivity with Other Human EGFR Ligands

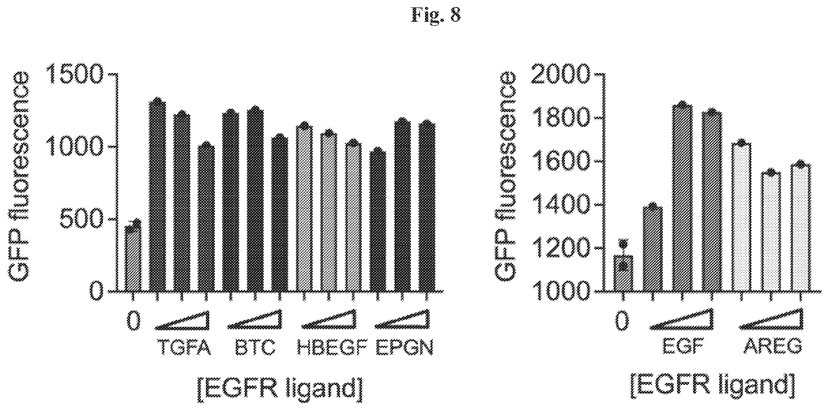

Next it was considered whether hEreg NAb1 could inhibit other EGFR ligands (commercially available from R&D Systems). HFF was incubated with 5 mg/ml of hEreg NAb1 alone, which as discussed above, inhibits epiregulin-driven growth of HFF. Whether addition of the other six EGFR ligands could rescue HFF growth through their activation of EGFR was tested. Compared to the hEreg NAb1 alone control, the lowest concentration tested of each EGFR ligand, 0.1 ng/ml, was sufficient to rescue HFF growth (FIG. 8). For EGF, 0.1 ng/ml showed intermediate rescue, and complete rescue by 1 ng/ml. In other experiments, this stock of recombinant EGF did not stimulate HFF proliferation below 1 ng/ml, so the intermediate rescue more likely reflects its relative affinity rather than cross-reactivity by the epiregulin antibody. Therefore, hEreg NAb1 does not cross-react with other EGFR ligands.

### Example 5. Humanized Epiregulin Antibody has Affinity Similar to FDA-Approved Biologic Medications

Octet Bio-Layer Interferometry was used to assess the kinetics of humanized epiregulin antibody binding to immobilized recombinant human epiregulin protein, hEreg NAb1 was found to have a K_{D} of 38×10⁻¹² along with an IC₅₀ of 1.8 nM calculated from HFF proliferation assays (Table 2). These are similar measurements to currently FDA-approved monoclonal antibody therapies for treatment of psoriasis.

TABLE 2
Kinetic measurements of hEreg NAb1 by Octet
Bio-Layer Interferometry and comparison to
FDA-approved monoclonal antibody medications

| Description | KD (×10⁻¹²) | IC₅₀ (nM) |
|---|---|---|
| hEreg NAb1 | 38 | 1.8 |
| Tocilizumab | 2500* | 0.09⁸⁰ |
| Adalimumab* | 58-87 | 0.1-1 |
| Rizankizumab* | 29 | 0.024 |

*data from European Medicines Agency

### Example 6. Use of hEreg NAbs for Treatment of Fibrotic Diseases

Enrichment of epiregulin was identified in human scleroderma skin and lung fibrotic tissue. In mouse models, rat anti-human/mouse epiregulin antibody was used as a surrogate therapeutic and it reversed both skin and lung fibrosis, hEreg NAb1 can be used to treat scleroderma-associated fibrosis of skin, lung and other tissues. Epiregulin enrichment occurs in other fibrotic tissues, such as idiopathic pulmonary fibrosis, keloids and scars, so hEreg Nabs may be of therapeutic potential for these diseases as well. Furthermore, lung fibrosis due to COVID19 has similar single cell RNA expression profile to idiopathic pulmonary fibrosis. Thus, hEreg NAbs may be broadly applicable for treatment of human fibrotic diseases in multiple tissues, also including chronic graft-vs-host disease, sclerotic graft-vs-host disease, sclerotic graft-vs-host disease skin fibrois, and others.

### Example 7, hEreg NAb1 Reduces Skin Fibrosis Markers

As described in the Odell et al, epiregulin Science Immunology manuscript, enrichment of epiregulin was identified in human scleroderma skin and lung fibrotic tissue. In bleomycin induced skin and lung fibrosis mouse models, a commercially available rat anti-human/mouse epiregulin antibody (R&D Biosciences MAB1068) reversed both skin and lung fibrosis. This antibody was tested for its ability to reduce skin fibrosis in human disease. This antibody was incubated with skin explants from a patient with SSc as well as a lung explants from a deceased donor with IPF compared to non-treated control. It was found that epiregulin inhibition with this surrogate antibody decreased expression of fibrosis protein markers in from both SSc fibrotic skin and IPF fibrotic lung.

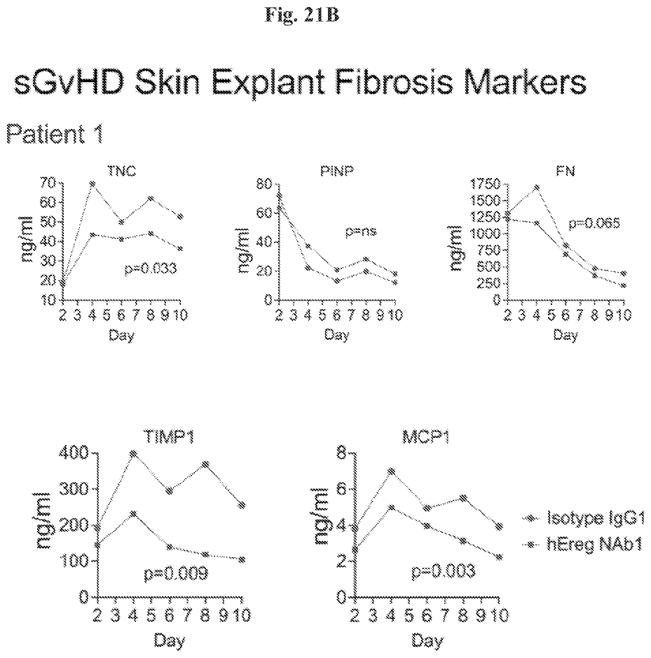

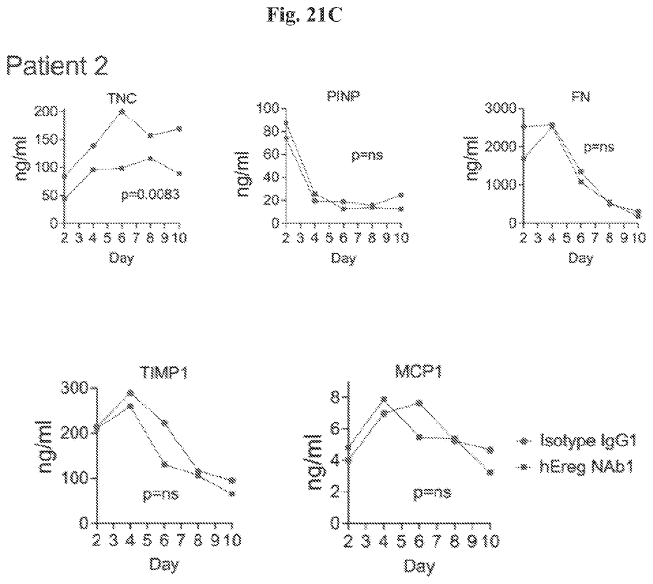

It was then investigated whether hEreg NAb1 could reduce fibrosis similar to the epiregulin neutralizing rat antibody. To investigate whether hEreg NAb1 reduces fibrosis protein markers, hEreg NAb1 was tested on skin biopsies from two patients with the fibrotic form of graft-vs-host disease (sGvHD). Patient I was a 51-year-old male 3 years post stem cell transplant from matched unrelated donor for treatment of myelodysplastic syndrome not on any immunosuppressive therapies. Patient 2 was a 76-year-old male 3 years post stem cell transplant from matched unrelated donor for treatment of acute myelogenous leukemia not on any immunosuppressive therapies. Paired 4 mm skin biopsies from the most affected skin of the right arm of patient 1 and left abdomen of patient 2 were obtained (FIG. 21A). Skin explants were incubated with 2.5 mg/ml hEreg NAb1 or isotype control IgG1 antibody for 10 days. Media was changed every 48 hours and the level of fibrosis protein markers in the supernatant was quantified by ELISA, hEreg NAb1-treated skin explant from patient 1 showed significantly reduced expression of TNC, TIMP1, and MCP-1 compared to IgG1 isotype control antibody (FIG. 21B-21C). From patient 2, hEreg NAb1-treated skin also showed significant reduction in TNC expression, but not TIMP 1 or MCP-1, hEreg NAb1 did not reduce expression of pro-collagen I alpha 1/pro-collagen I N-terminal propeptide (PINP) or fibronectin (FN) in skin explants from either patient. These results demonstrate that hEreg NAb1 can directly reduce expression of multiple fibrosis markers by human fibrotic skin. Because TNC was the most consistently reduced fibrosis marker gene, it may provide a good biomarker for hEreg NAb1 in patients.

### Example 8, hEreg NAb1 Indications

hEreg NAb1 has the strongest preclinical data for treatment of sGvHD, SSc, and IPF because hEreg NAb1 was validated for its ability to reduce sGvHD skin fibrosis and used a surrogate rat epiregulin neutralizing antibody in SSc skin fibrosis and IPF lung fibrosis, hEreg NAb1 has therapeutic potential for additional fibrotic diseases such as keloid scars. Furthermore, lung fibrosis due to COVID19 has similar single cell RNA expression profile to idiopathic pulmonary fibrosis. Thus, hEreg NAbs may be broadly applicable for treatment of human fibrotic diseases in multiple tissues.

### Example 9. EGFR Activation Marks Pathogenic Fibroblasts in SSc Skin and Lung

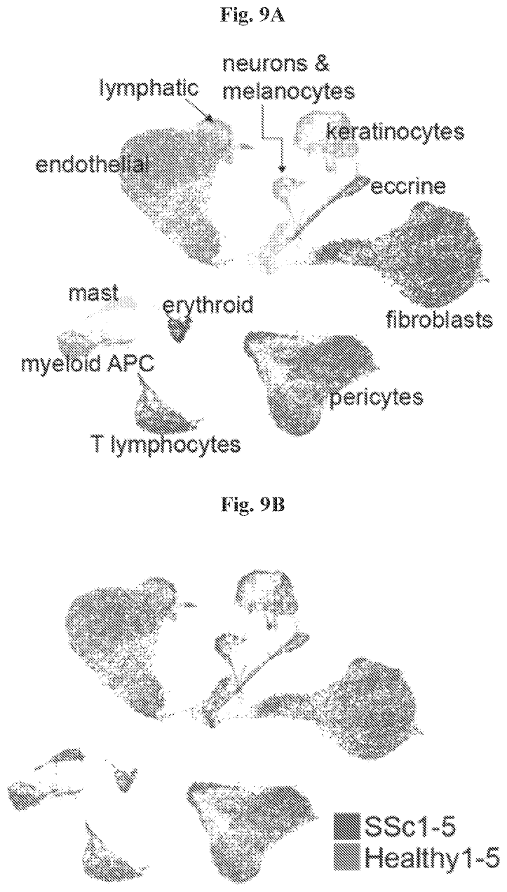

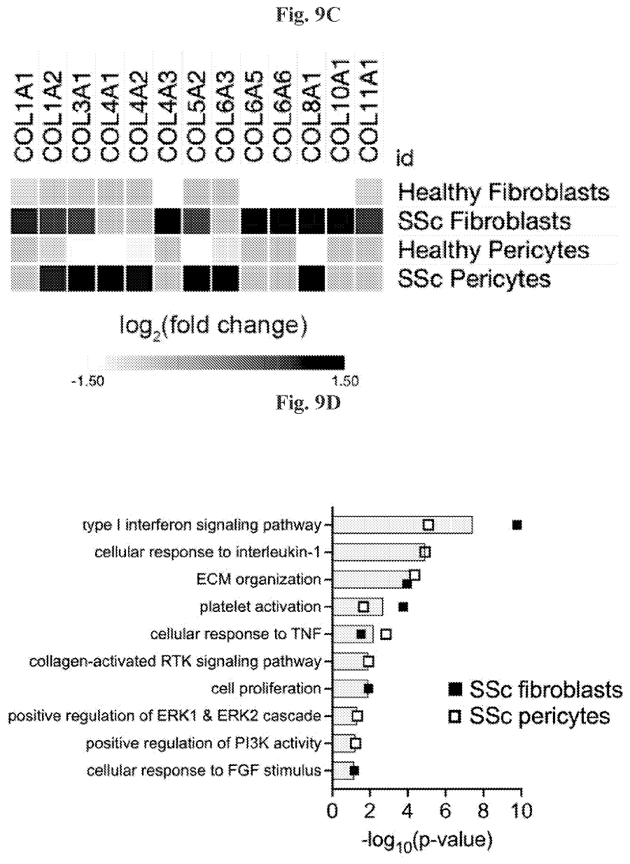

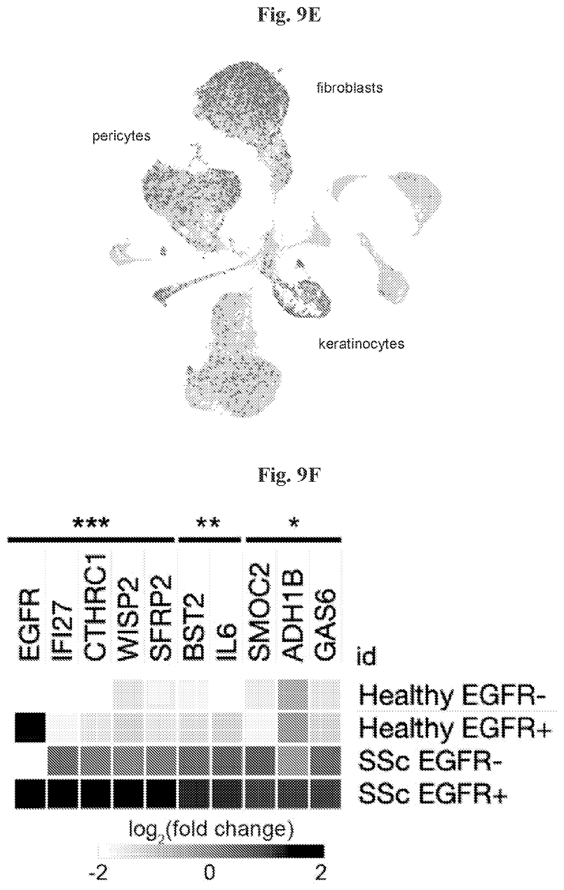

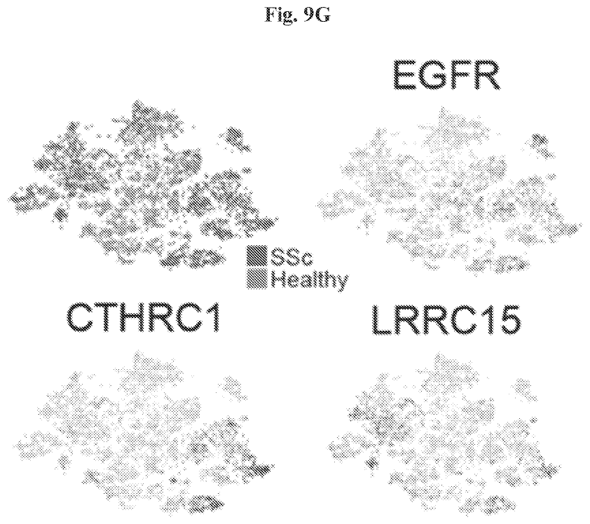

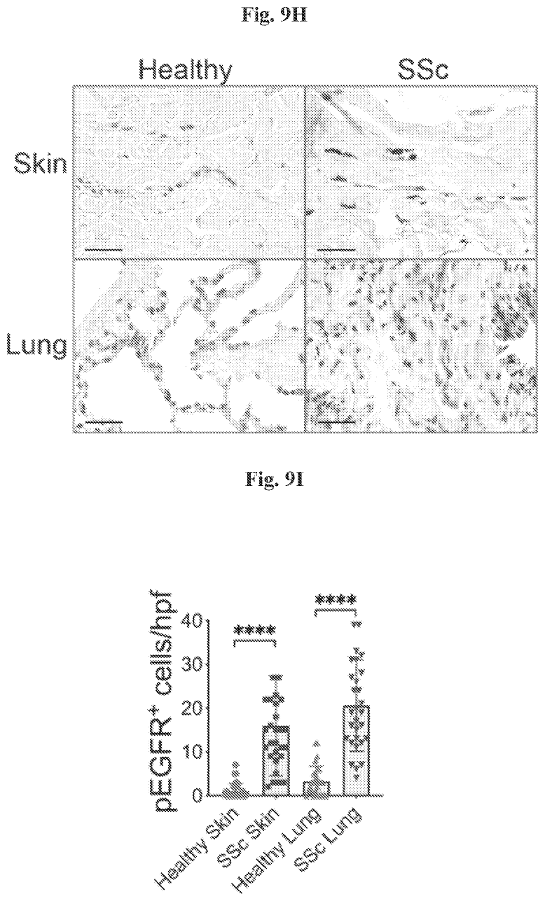

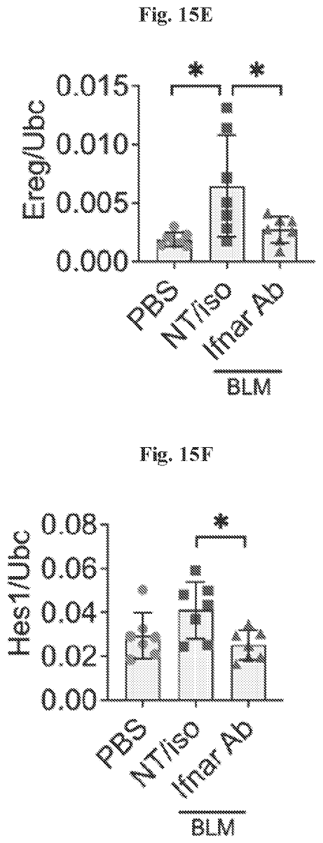

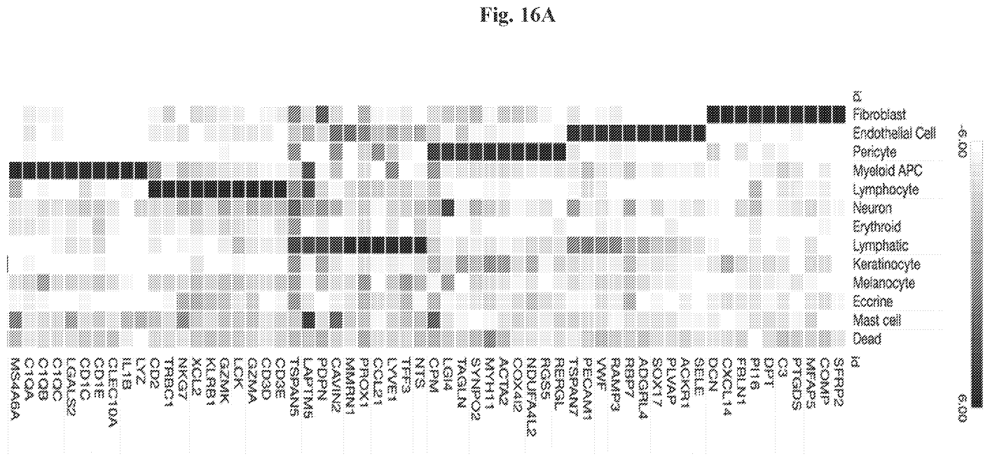

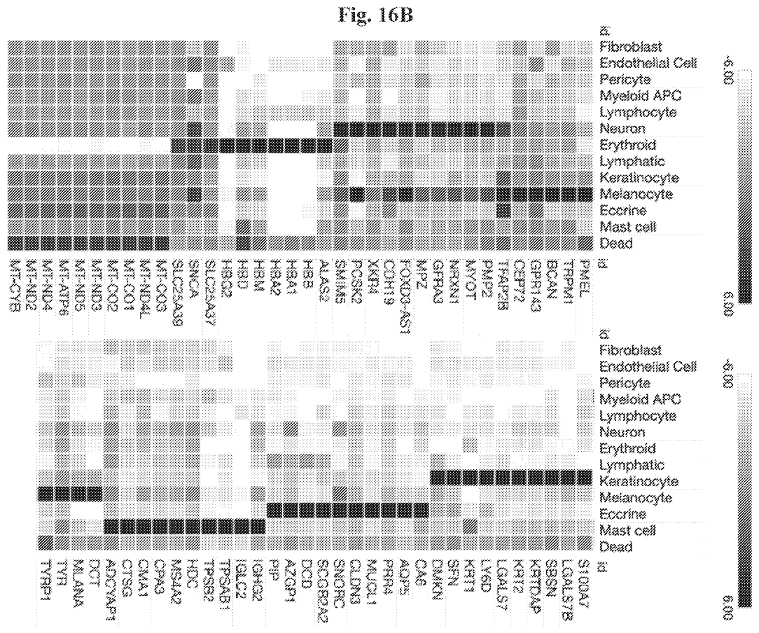

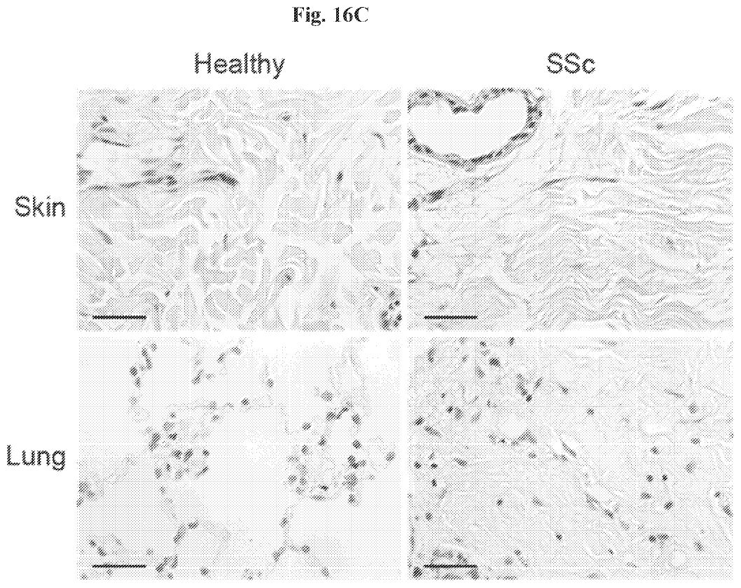

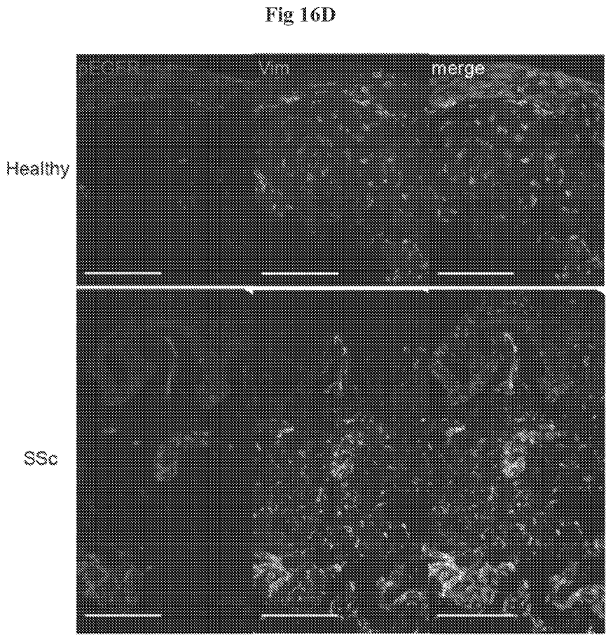

To understand the cellular signaling that occurs in SSc skin, biopsies of fibrotic skin were obtained from five patients with diffuse cutaneous SSc and five healthy controls for single-cell RNA sequencing (scRNA-Seq). Clinical characteristics of the study participants including age, sex, affected organs, comorbid conditions and immunomodulatory therapies at the time of biopsy are listed in Table 3. Immediately after skin biopsy, the tissue was digested, all live cells were sorted, and the 10× Chromium Single Cell Controller was used to create barcoded single-cell cDNA libraries. Uniform Manifold Approximation and Projection (UMAP) embedding of the single cell cDNA identified 12 major cell clusters (FIG. 9A) each defined by a set of signature genes (FIG. 16). Cells from individual SSc samples localized in similar regions of the fibroblast, pericyte and endothelial clusters (FIG. 9B), suggesting similar gene expression profile in the cells from fibrotic skin compared to those from healthy skin. SSc dermal fibroblasts showed increased expression of multiple extracellular matrix (ECM) genes, including types I and III collagens (FIG. 9C). Similarly. SSc pericytes, marked by their expression of regulator of G protein signaling 5 (RGS5) (26), showed increased expression of collagen I, III, IV, and VI transcripts. Together, these data show that both fibroblasts and pericytes produced excessive collagen in SSc skin.

TABLE 3
Select clinical characteristics of the patients in this study

| | | | | | Past | | | Immuno- |
|---|---|---|---|---|---|---|---|---|
| | Age | Sex | | Duration | Medical | Other Organ | Auto- | modulating |
| Sample | (years) | (M/F) | Ethnicity | (year) | History | Involvement | Abs | Medications |
| SSc1 | 60 | M | W | 0.33 | | Renal | RNA Pol | None |
| | | | | | | | III | |
| SSc2 | 61 | M | W | 22 | | None | Negative | ECP |
| | | | | | | | serologies | |
| SSc3 | 39 | M | W | 4 | | Interstitial | ANA | Bosentan |
| | | | | | | lung disease | 1:2560 | ECP |
| | | | | | | esophageal | | |
| | | | | | | dysmotility | | |
| SSc4 | 57 | F | W | 2 | IBS | Inflammatory | ANA | Myco- |
| | | | | | | arthritis | 1:320 | phenolate |
| | | | | | | | RNA Pol | mofetil |
| | | | | | | | III | phototherapy |
| | | | | | | | CCP | prednisone |
| | | | | | | | | hydroxy- |
| | | | | | | | | chloroquine |
| SSc5 | 47 | F | W | 10 | | Interstitial | ANA | Myco- |
| | | | | | | lung disease | 1:320 | phenolate |
| | | | | | | pulmonary | SSA | mofetil |
| | | | | | | arterial | RNP | hydroxy- |
| | | | | | | hypertension | | chloroquine |
| | | | | | | | | sildenafil |
| SSc6 | 54 | F | W | 0.75 | | Interstitial | ANA | Myco- |
| | | | | | | lung disease | 1:320 | phenolate |
| | | | | | | GERD | RNA Pol | mofetil |
| | | | | | | | III | |
| Normal1 | 24 | F | W | | None | | | |
| Normal2 | 35 | M | W | | Dermo- | | | |
| | | | | | graphism | | | |
| Normal3 | 54 | M | W | | None | | | |
| Normal4 | 52 | M | W | | BCC | | | |
| Normal5 | 38 | M | W | | None | | | |

Abbreviations:
W, white;
M, male;
F, female;
GERD, gastroesophageal reflux disease;
ECP, extracorporeal photopheresis;
BCC, basal cell carcinoma;
IBS, inflammatory bowel disease;
Abs, Antibodies;
ANA, antinuclear antibodies;
CCP, cyclic citrullinated peptide;
RNA Pol III, RNA polymerase III

To identify signaling pathways that drive higher ECM production in SSc, the differential gene expression of each cell type in SSc compared to healthy controls was calculated and functional enrichment analysis using DAVID (27) of upregulated genes with log 2 (fold change)>0.58 and p-value<0.05 was performed. As shown in FIG. 9D (full list displayed in Table 4), enriched gene ontology terms in SSc fibroblasts and pericytes versus healthy controls included results such as ECM organization and type I interferon signaling (28, 29). However, in addition to these results, multiple pathways associated with receptor tyrosine kinase (RTK) signal transduction (e.g., cell proliferation, response to fibroblast growth factor (FGF) stimulus, collagen-activated RTK signaling, positive regulation of extracellular signal-regulated kinase 1 and 2 (ERK1 and ERK2) cascade, and positive regulation of phosphoinositide 3-kinase (PI3K) activity) were found. Based on previous studies, which showed that inhibition of EGFR could prevent fibrosis (8-10), and the finding herein that multiple EGFR ligands are overexpressed in SSc skin (see also, e.g., Example 10), the scRNA-Seq data were interrogated to determine the cell types that most highly express EGFR, revealing that EGFR is expressed by fibroblasts and pericytes (FIG. 9E). Thus, these data suggest that EGFR is activated in SSc fibroblasts and pericytes and may regulate their ECM production. Table 4 shows healthy and SSc skin and lung enriched in receptor-ligand interactions. In particular, enriched receptor ligand interactions among the SSc skin data and at least 2/3 SSc lung datasets were compared to their healthy controls. Interactions shown in italic text are enriched in both skin and lung, whereas the remaining enriched interactions are dissimilar between skin and lung.

TABLE 4
Healthy and SSc skin and lung enriched receptor-ligand interactions

| SSc ILD2/3 | rank | SSc skin | rank | | rank | | rank |
|---|---|---|---|---|---|---|---|
| PDGFR | 0.004 | EREG_{—}EGFR | 0.012 | L1CAM_a5b1 | 0.024 | MIF_TNFRSF10D | 0.041 |
| complex | | | | complex | | | |
| PDGFD | | | | | | | |
| IGF2_{—}IFG1R | 0.004 | PDGFR | 0.024 | EPHB4_EFNB1 | 0.024 | COL5A2_a11b1 | 0.047 |
| | | complex_{—}PDGFD | | | | complex | |
| PDGFRB_{—}PDGFD | 0.009 | BMPR1A_{—} | 0.024 | ACVR_1B2A | 0.024 | COL1A2_a11b1 | 0.047 |
| | | BMPR2_{—}BMP4 | | receptor_INHBA | | complex | |
| BMPR1A_{—} | 0.009 | IGF1_{—}IGF1R | 0.024 | COL9A2_a11b1 | 0.024 | FN1_a3b1 | 0.047 |
| BMPR2_{—}BMP4 | | | | complex | | complex | |
| IGF1_{—}IGF1R | 0.018 | FLT1 | 0.036 | EPHB3_EFNB1 | 0.024 | PDGFA_{—} | 0.047 |
| | | complex_{—}PGF | | | | PDGFRA | |
| EREG_{—}EGFR | 0.018 | EGFR_{—}HBEGF | 0.036 | COL9A3_a11b1 | 0.024 | COL6A1_a11b1 | 0.047 |
| | | | | complex | | complex | |
| EGFR_{—}AREG | 0.027 | CCL2_{—}CCR10 | 0.036 | COL1A1_a10b1 | 0.024 | COL1A1_a11b1 | 0.047 |
| | | | | complex | | complex | |
| EGFR_{—}HBEGF | 0.036 | IGF2_{—}IGF1R | 0.036 | TNFSF10_RIPK1 | 0.024 | COL12A1_a11b1 | 0.047 |
| | | | | | | complex | |
| FLT1 | 0.036 | PDGFRB_{—}PDGFD | 0.041 | CADM3_CADM1 | 0.024 | NRP1_VEGFB | 0.047 |
| complex_{—}PDF | | | | | | | |
| JAG1_{—}NOTCH4 | 0.044 | JAG1_{—}NOTCH4 | 0.041 | EPHA4_EFNB1 | 0.024 | TNF_PTPRS | 0.047 |
| CCL2_{—}CCR10 | 0.044 | EGFR_{—}AREG | 0.047 | COL6A1_a10b1 | 0.024 | COL16A1_a211b1 | 0.047 |
| | | | | complex | | complex | |
| BMPR1A_BMPR2_{—} | 0.003 | COL28A1_a10b1 | 0.006 | OSMR_OSM | 0.024 | PROS1_TYRO3 | 0.047 |
| BMP6 | | complex | | | | | |
| BMPR1B_BMPR2_{—} | 0.003 | COL17A1_a10b1 | 0.006 | COL16A1_a10b1 | 0.024 | | |
| BMP6 | | complex | | complex | | | |
| BMR1B_AVR2A_{—} | 0.003 | EFNB2_EPHB3 | 0.006 | NRP1_SEMA3A | 0.024 | | |
| BMP6 | | | | | | | |
| BMPR1B_BMPR2_{—} | 0.003 | CD27_CD70 | 0.006 | COL8A1_a11b1 | 0.024 | | |
| BMPR1A | | | | complex | | | |
| BMR1A_ACR2A_{—} | 0.004 | EFNB2_EPHB4 | 0.006 | NOTCH1_NOV | 0.024 | | |
| BMP5 | | | | | | | |
| AGT_AGTR1 | 0.004 | KDR_VEGFC | 0.006 | SELL_CD34 | 0.024 | | |
| BMPR1A_BMPR2_{—} | 0.004 | L1CAM_L1CAM | 0.006 | SELP_CD34 | 0.024 | | |
| BMP5 | | | | | | | |
| BMPR1A_BMPR2_{—} | 0.004 | CTLA4_CD86 | 0.006 | COL1A2_a10b1 | 0.024 | | |
| BMP2 | | | | complex | | | |
| NOTCH3_JAG2 | 0.009 | COL4A5_a10b1 | 0.006 | CCL21_ACKR4 | 0.024 | | |
| | | complex | | | | | |
| BMR1A_ACR2A_{—} | 0.009 | ICOSLG_ICOS | 0.006 | COL6A6_a11b1 | 0.024 | | |
| BMP4 | | | | complex | | | |
| COL13A1_2b1 | 0.009 | BMP7_SLAMF1 | 0.006 | COL12A1_a10b1 | 0.024 | | |
| complex | | | | complex | | | |
| DLL4_NOTCH3 | 0.009 | COL17A1_a11b1 | 0.012 | NAMPT_P2RY6 | 0.03 | | |
| | | complex | | | | | |
| JAG1_NOTCH3 | 0.012 | FLT1 | 0.012 | PTN_PTPRZ1 | 0.03 | | |
| | | complex_VEGFB | | | | | |
| FGFR2_XPR1 | 0.013 | COL8A1_a10b1 | 0.012 | CXCL8_ACKR1 | 0.03 | | |
| | | complex | | | | | |
| PVR_CD226 | 0.013 | TNF_RIPK1 | 0.012 | COL18A1_a10b1 | 0.03 | | |
| | | | | complex | | | |
| TNC_a9b1 | 0.013 | COL4A5_a11b1 | 0.012 | COL21A1_a10b1 | 0.03 | | |
| complex | | complex | | complex | | | |
| COL5A3_a2b1 | 0.018 | SELL_SELPLG | 0.012 | COL15A1_a10b1 | 0.03 | | |
| complex | | | | complex | | | |
| CCR10_CCL7 | 0.018 | ACVR_1B2A | 0.012 | COL14A1_a10b1 | 0.03 | | |
| | | receptor_GDF11 | | complex | | | |
| NOTCH4_JAG2 | 0.018 | COL9A3_a10b1 | 0.012 | ANGPT2_TEK | 0.03 | | |
| | | complex | | | | | |
| NOTCH1_JAG2 | 0.018 | COL28A1_a11b1 | 0.012 | AXL_IL15RA | 0.03 | | |
| | | complex | | | | | |
| COL21A1_a2b1 | 0.018 | COL9A2_a10b1 | 0.012 | COL4A1_a10b1 | 0.03 | | |
| complex | | complex | | complex | | | |
| COL5A3_a10b1 | 0.018 | ACKR1_CCL17 | 0.012 | HLA-F_{—} | 0.03 | | |
| complex | | | | LILRB1 | | | |
| COL8A2_a2b1 | 0.018 | COL6A6_a10b1 | 0.012 | COL4A2_a10b1 | 0.03 | | |
| complex | | complex | | complex | | | |
| VCAM1_a9b1 | 0.018 | CCR10_CCL27 | 0.012 | COL6A2_a10b1 | 0.03 | | |
| complex | | | | complex | | | |
| EGFR_GRN | 0.022 | CSPG4_a3b1 | 0.012 | CMKLR1_RARRES2 | 0.03 | | |
| | | complex | | | | | |
| COL5A3_a1b1 | 0.027 | CD70_GPRC5B | 0.012 | COL6A3_a11b1 | 0.036 | | |
| complex | | | | complex | | | |
| PLXNB1_SEMA4D | 0.027 | CADM3_EPB41L1 | 0.012 | TYRO3_GAS6 | 0.036 | | |
| COL8A2_a1b1 | 0.027 | CADM3_CADM4 | 0.012 | COL28A1_a1b1 | 0.036 | | |
| complex | | | | complex | | | |
| TGFB3_TGFBR3 | 0.027 | PODXL_SELL | 0.012 | COL4A5-a1b1 | 0.036 | | |
| | | | | complex | | | |
| CD8 | 0.027 | EPHB6_EFNB2 | 0.018 | COL17A1_a1b1 | 0.036 | | |
| receptor_LCK | | | | complex | | | |
| THY1_aXb2 | 0.028 | COL5A2_a10b1 | 0.018 | IGF1_a6b4 | 0.036 | | |
| complex | | complex | | complex | | | |
| COL15A1_a10b1 | 0.036 | COL3A1_a10b1 | 0.018 | IGF2_IGF2R | 0.036 | | |
| complex | | complex | | | | | |
| THY1_aMb2 | 0.037 | NOTCH2_DLL4 | 0.018 | COL3A1_a11b1 | 0.036 | | |
| complex | | | | complex | | | |
| TNF_DAG1 | 0.044 | ACVR_1B2A | 0.018 | FN1_a10b1 | 0.036 | | |
| | | receptor_INHBB | | complex | | | |
| NOTCH1_JAG1 | 0.044 | COL5A3_a10b1 | 0.018 | COL5A1_a11b1 | 0.036 | | |
| | | complex | | complex | | | |
| LAMC1_a2b1 | 0.044 | COL5A1_a10b1 | 0.018 | FLT1_PGF | 0.036 | | |
| complex | | complex | | | | | |
| NRP2_SEMA3C | 0.046 | LIFR_OSM | 0.018 | CXADR_FAM3C | 0.041 | | |
| COL15A1_a1b1 | 0.049 | FLT3_FLT3LG | 0.018 | MDK_PTPRZ1 | 0.041 | | |
| complex | | | | | | | |

Determination of whether EGFR-expressing (EGFR⁺) fibroblasts and pericytes show differential expression of relevant SSc gene signatures was a key focus of the present Example. Using skin scRNA-Seq data, differential gene expression of EGFR⁺ SSc fibroblasts compared to EGFR-SSc fibroblasts and EGFR⁺ and EGFR-healthy control fibroblasts was calculated.

The upregulated genes with the highest differential expression and lowest p-value by EGFR⁺ SSc fibroblasts compared to the other fibroblast subsets are shown in FIG. 9F. In particular, EGFR⁺ SSc fibroblasts expressed higher levels of genes associated with wingless-related integration site (Wnt) signaling (e.g., secreted frizzled related protein 2, SFRP2; collagen triple helix repeat containing 1, CTHRC1; and, Wnt-1-inducible signaling pathway protein-2, WISP2), a pathway activated by fibroblasts that mostly reside in the reticular dermis (13) and is hyperactivated in SSc skin (30). Among Wnt genes, SFRP2 expression defines a subset of dermal fibroblasts (31, 32) that were recently shown to differentiate into myofibroblasts in SSc skin (15). CTHRC1 is a gene that characterizes a fibroblast subpopulation that produces the most collagens in SSc and idiopathic pulmonary fibrosis (IPF) fibrotic lung specimens (33) and is a marker along with leucine rich repeat containing 15 (LRRC15) for myofibroblasts across tissues (16). EGFR⁺ SSc fibroblasts also expressed higher levels of interferon inducible genes (e.g., interferon alpha-inducible protein 27 (IFI27), bone marrow stromal cell antigen 2 (BST2), interferon alpha-inducible protein 6 (IFI6)), which have been shown to correlate with SSc disease severity (34, 35). Fibroblast subclustering shown in FIG. 9G demonstrates that both healthy and SSc fibroblasts express EGFR; however, in SSc fibroblasts, EGFR co-expresses with the fibrosis signature genes described above. In contrast to EGFR⁺ fibroblasts, similar analysis of EGFR⁺ pericytes did not reveal overexpression of fibrotic gene signatures. Thus, EGFR⁺ SSc fibroblasts, but not pericytes, express multiple key gene signatures associated with SSc skin and lung fibrosis.

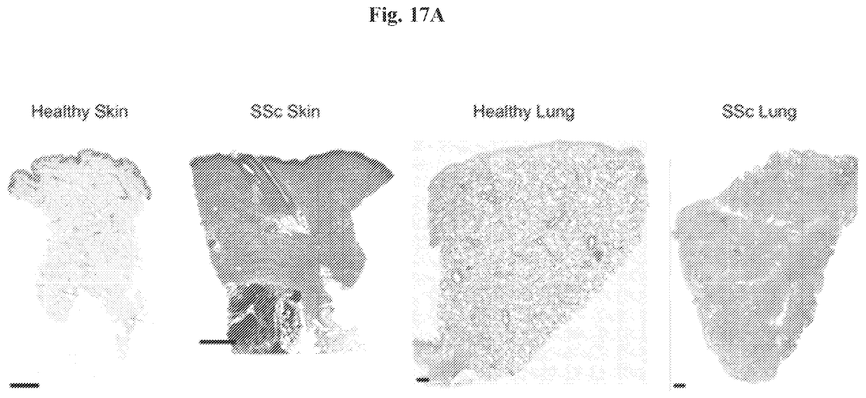

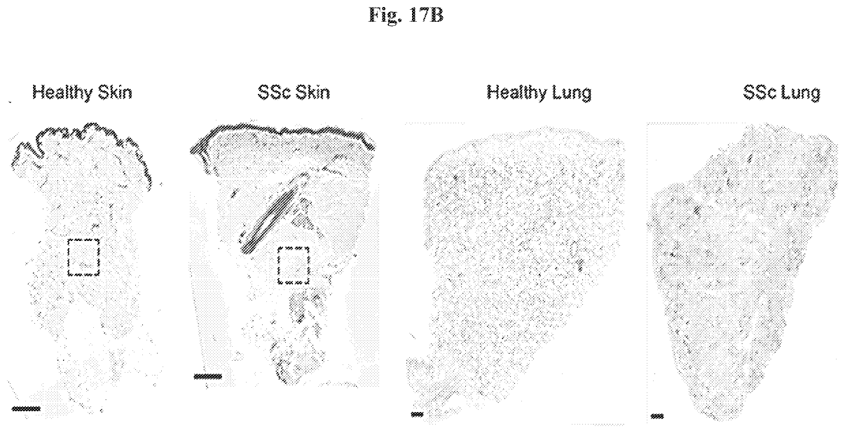

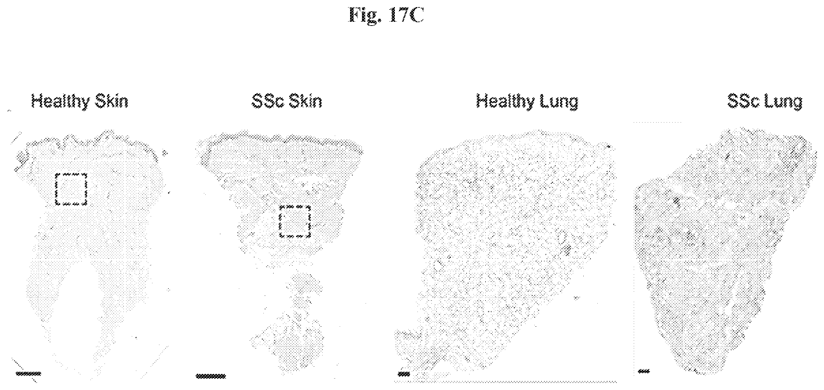

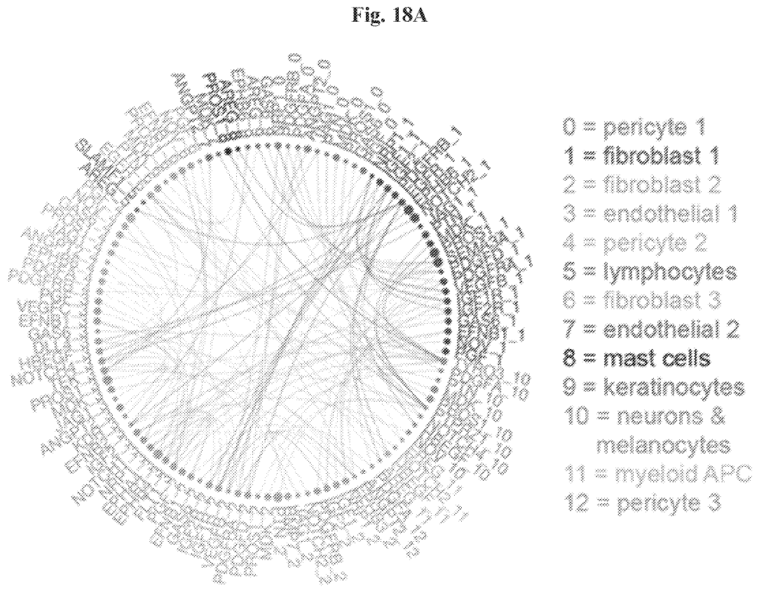

If EGFR⁺ SSc fibroblasts are activated in regions of fibrotic dermis was evaluated next. Upon activation, EGFR becomes phosphorylated at multiple sites on its cytoplasmic domains (36), detectable using phospho-specific antibodies to phosphorylate EGFR (pEGFR). To confirm activation of EGFR in SSc. SSc skin was stained with a phospho-specific antibody against EGFR Tyr-1068 and strongly labelled cells were observed in fibrotic dermis (FIG. 9H top row, with isotype controls in FIG. 16C and lower magnification scans of histology and immunohistochemistry in FIG. 17). Similarly, pEGFR⁺ cells were observed in SSc fibrotic lung tissue, as shown in the bottom row of FIG. 9H. Both vascular and individual pEGFR⁺ cells in the dermis co-stained with vimentin (FIG. 16D), supporting its expression and activation in fibroblasts and pericytes. Quantification of pEGFR⁺ cells in skin and lung showed that they were significantly increased in SSc versus healthy control samples (FIG. 9I). Therefore, EGFR-expressing fibroblasts are activated in fibrotic skin and lung and express genes that characterize fibrosis.

### Example 10. Epiregulin⁺ Dendritic Cells Accumulate in Human Skin and Lung Fibrosis

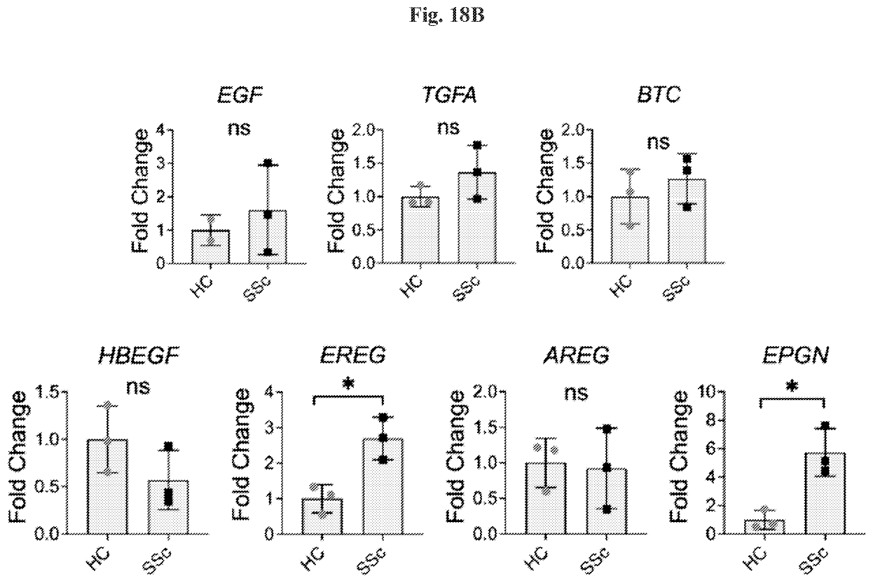

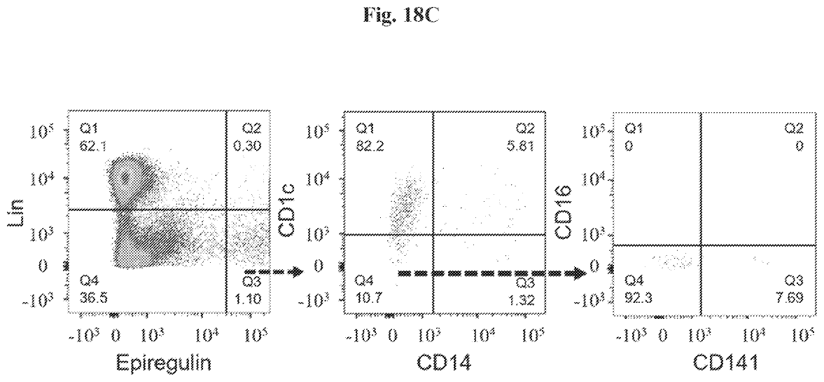

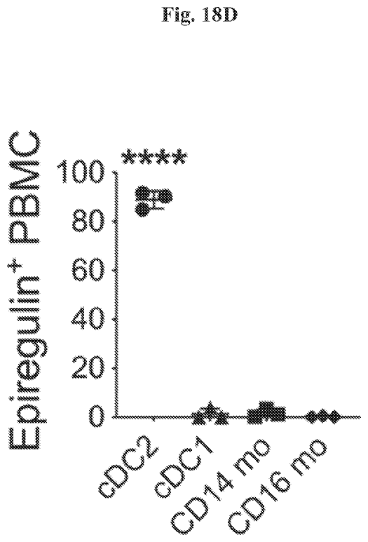

EGFR has seven activating ligands with different signaling properties based on their binding kinetics (37, 38). To characterize activating ligands of EGFR and other RTK in SSc skin, receptor-ligand enrichment in the skin scRNA-Seq dataset was identified using CellphoneDB (39, 40), which identifies increased expression of receptor-ligand pairs between cell clusters (www.cellphonedb.org). Significant interactions as indicated by rank <0.05 in SSc vs healthy skin were identified and examined based on the cell types producing each ligand-receptor pair. Enriched interactions in the SSc skin signaling network were largely comprised of growth factor ligands and their receptors (depicted in FIG. 18A), including EGFR, platelet-derived growth factor receptor (PDGFR), NOTCH, Ephrins, and other receptor tyrosine kinases, with most growth signals occurring between mesenchymal cell types. Compared to mesenchymal cells, immune cells expressed distinct growth factor ligands including EGFR activating ligands and oncostatin M. These findings suggest that immune-mesenchymal and mesenchymal-mesenchymal growth factor signals support the cell-cell communication network in fibrotic skin.

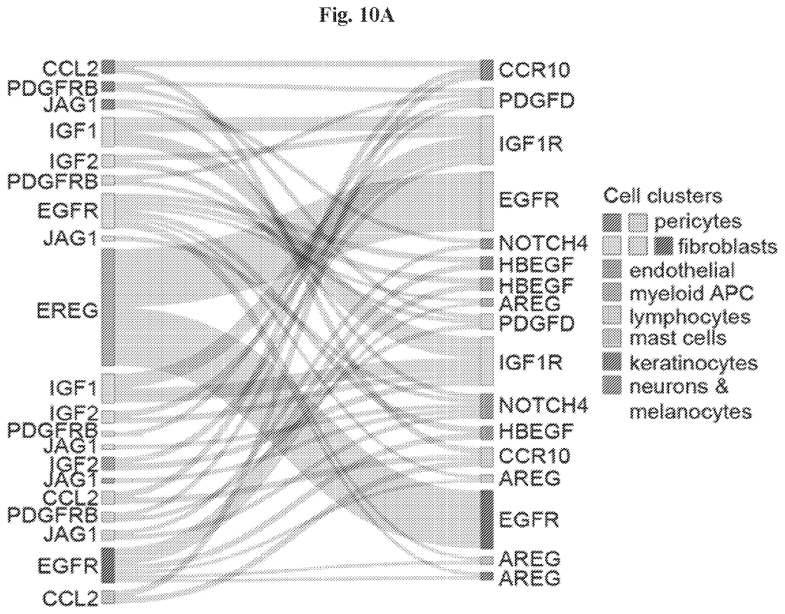

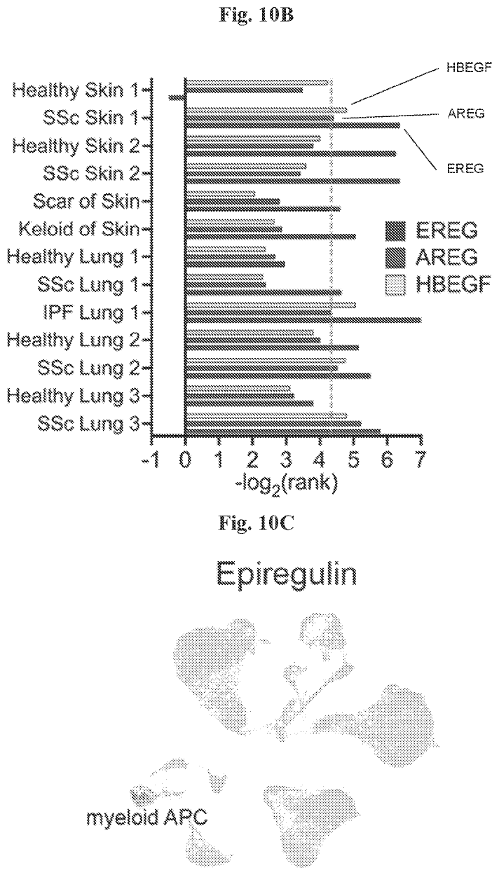

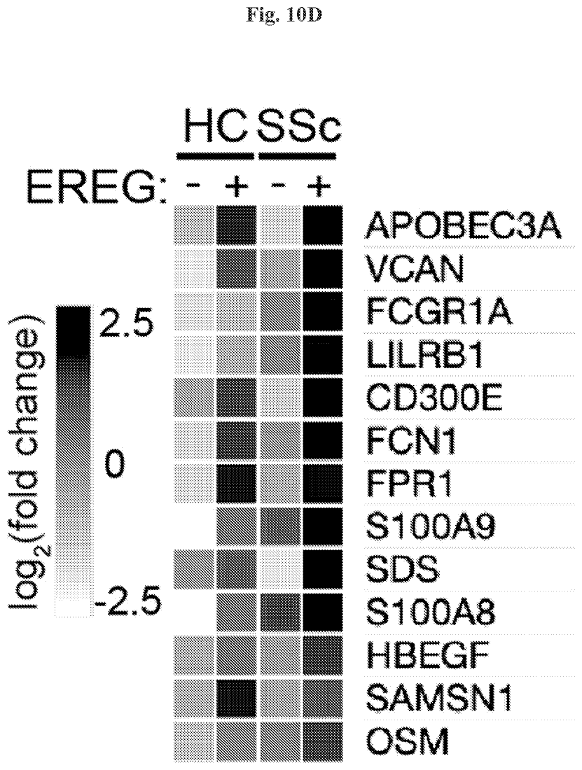

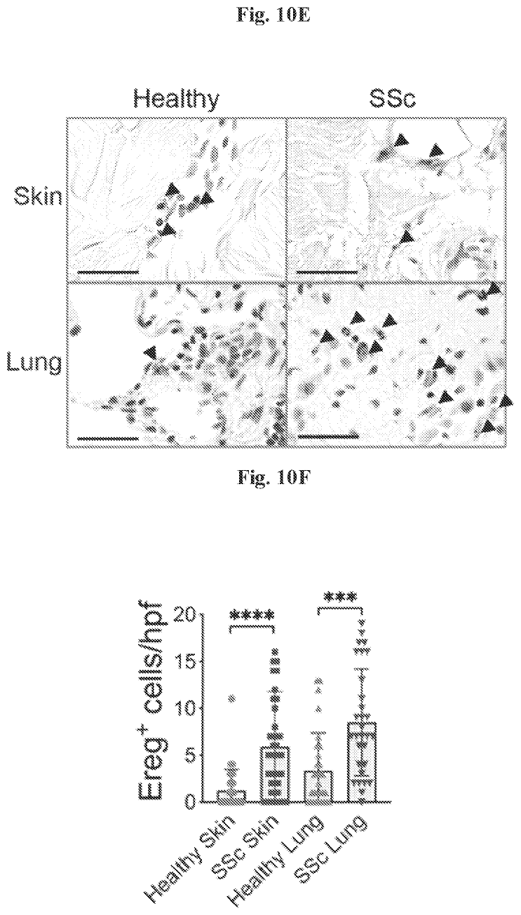

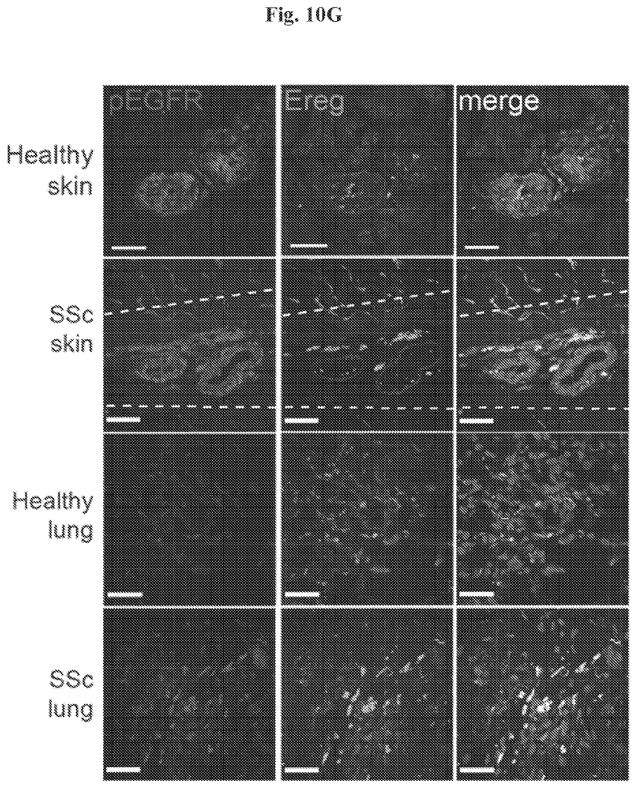

To investigate whether important interactions identified in SSc skin are reproducible in other tissues, the skin scRNA-Seq data were compared to publicly available scRNA-Seq data from another study of SSc skin (15), a study of keloid scars in the skin (14), and three studies of SSc-associated pulmonary fibrosis of patients at the time of lung transplant (33, 41, 42). Enriched interactions common to SSc skin and at least two of the lung datasets are diagramed in FIG. 10A. The most significant interaction was found between epiregulin from the cluster of myeloid antigen presenting cells (APC) and EGFR in two subclusters of fibroblasts. Elevated expression of epiregulin in whole tissue of SSc skin compared to healthy controls was confirmed by quantitative polymerase chain reaction (qPCR) (FIG. 18B). Among myeloid antigen-presenting cell (APCs), which includes dendritic cells (DCs), monocytes and macrophages, expression of the EGFR ligands amphiregulin and heparin-binding EGF-like growth factor (HBEGF) were also enriched in the scRNA-Seq data, but not increased when as saved by qPCR. To better visualize the hierarchy of myeloid APC-fibroblast interactions, the rank of each EGFR ligand from each study was plotted in FIG. 10B. Compared to healthy controls, SSc skin and lung showed more enrichment of the epiregulin-EGFR interaction than amphiregulin or HBEGF. Enrichment of epiregulin with EGFR was also observed in scarred and keloidal skin, and the lungs of patients with idiopathic pulmonary fibrosis (IPF), a related but pathologically distinct fibrotic lung disease. Two studies (FIG. 10C skin and lung 2) also showed enriched expression of epiregulin with EGFR in healthy tissue, which may be due to a technical artifact of sample processing for longer digestion time. Altogether, these data suggest that epiregulin is a common pathogenic signaling molecule in multiple fibrotic diseases of the skin and lung.

The cellular origin and function of epiregulin has not clearly been defined. Among cell types in the skin scRNA-Seq data, epiregulin was uniquely expressed in the cluster of myeloid APC (FIG. 10C), whereas amphiregulin and HBEGF were expressed by myeloid APC and multiple other cell types. In a recent study of healthy human lung, epiregulin was identified as a defining gene specific for a rare DC cell population (43). In the skin data described herein, epiregulin-expressing (EREG⁺) APC from both healthy and SSc samples showed a similar gene expression profile between each other as well as co-expression of amphiregulin similar to healthy human lung (FIG. 10D), EREG⁺ APC also showed elevated markers of DC3 (e.g., versican (VCAN), S100A8 and S100A9), an inflammatory subset of circulating cluster of differentiation 1c positive (CD1c⁺) DC whose maturation can be induced by type I interferon (44, 45). Surface expression of epiregulin on circulating CD1c⁺ DC was confirmed by fluorescence-activated cell sorting (FACS) analysis of peripheral blood of healthy volunteers (FIGS. 18C-18D). Therefore, the enrichment of epiregulin expression among APC in SSc most likely reflects an inducible cellular state of DC3.

To assess the spatial localization and abundance of EREG⁺ DC in SSc skin and lung, their location was studied by immunohistochemistry and immunofluorescence. In SSc skin and lung, clusters of EREG⁺ DC were observed perivascularly and in regions of fibrotic tissue that were scarce in healthy skin and lung (FIG. 10E). Moreover, enumeration of EREG⁺ DC from SSc skin and lung showed significantly increased numbers of epiregulin-positive cells in each tissue compared to healthy controls (FIG. 10F). By immunofluorescence, both healthy and SSc skin showed pEGFR⁺ blood vessels and in SSc, these vessels coursed through fibrotic dermis and colocalized with epiregulin (FIG. 10G, upper rows). In SSc, but absent in healthy lung, were regions of fibrosis contained pEGFR⁺ cells that largely co-labeled with epiregulin (FIG. 10G, bottom rows). As epiregulin is shed from the cell surface through proteolytic cleavage by A disintegrin and metalloprotease 17 (ADAM17) (46), these findings suggest that in SSc skin and lung, secreted epiregulin binds EGFR on fibroblasts. Thus. EREG⁺ DC localize to SSc fibrotic skin and lung and are increased in abundance in both tissues, suggesting they could be responsible for activating EGFR to drive disease.

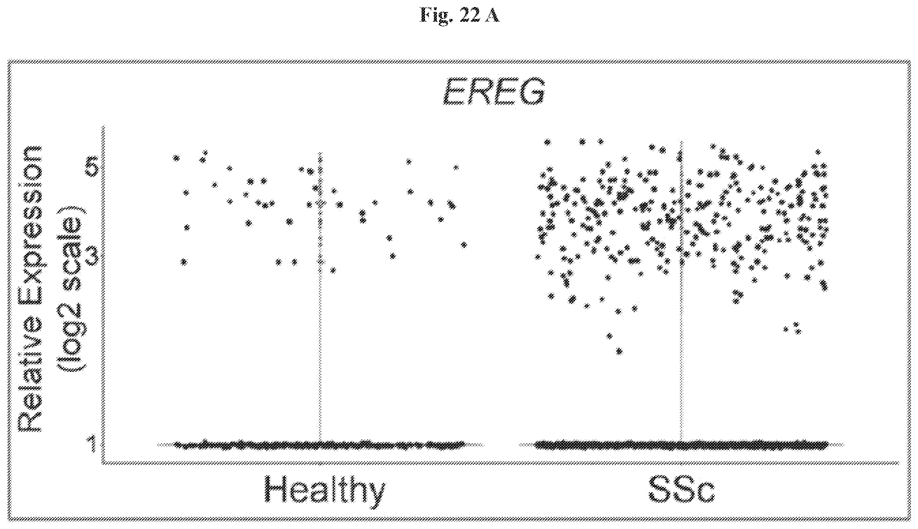

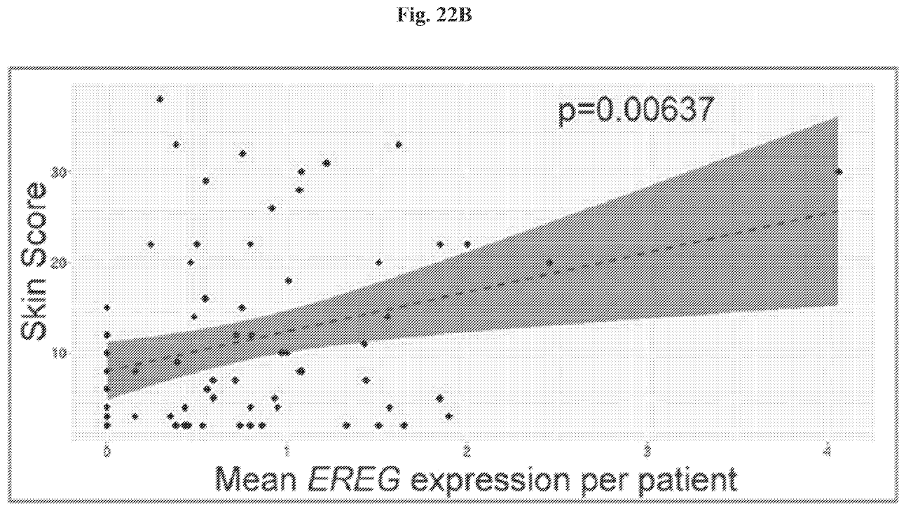

An increased number of EREG+ cells in SSc skin compared to healthy controls were found (FIG. 22A) and EREG expression showed a significant positive correlation with disease severity by modified Rodnan Skin Score (mRSS) (FIG. 22B). The strength of the correlation between EREG expression and mRSS was small to medium (Pearson correlation coefficient=0.31). This correlation is likely underestimated because the longer skin digestion time (1 hour) they used in their protocol induces EREG expression. Nevertheless. EREG expression by DC3 is associated with severity of skin fibrosis in SSc.

### Example 11. Epiregulin has Defined Expression Patterns in Mouse Skin and Lung Fibrosis

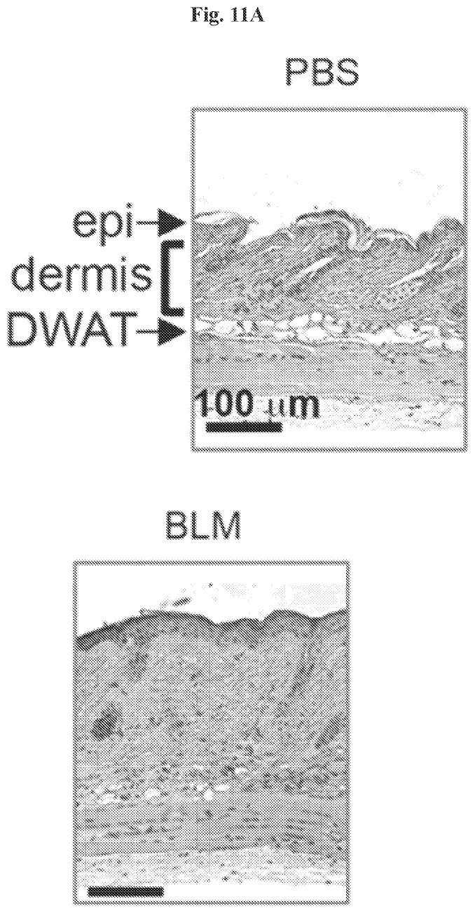

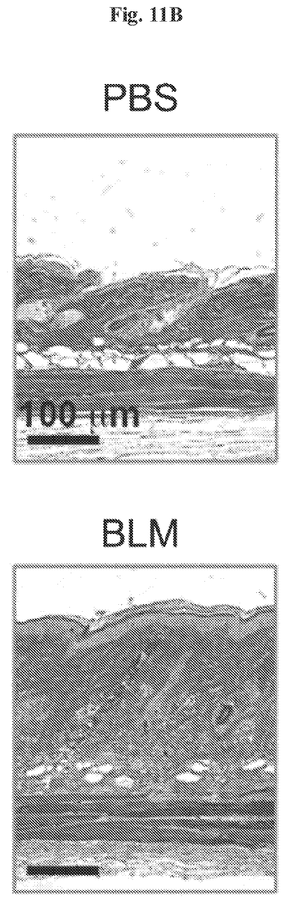

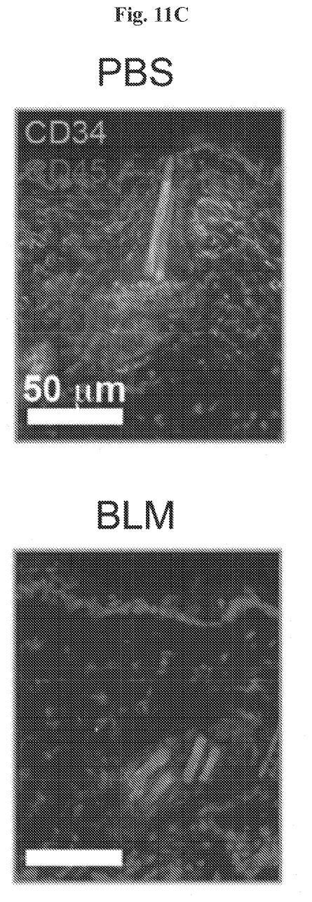

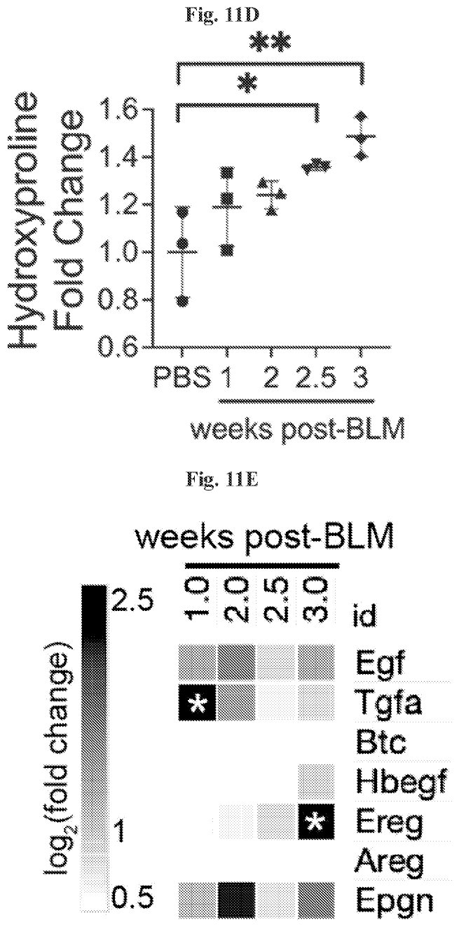

Different mouse models of SSc recapitulate characteristics of distinct SSc disease subsets (47). To best interrogate the role of epiregulin in SSc, the present Example required a mouse model that depends on myeloid APC and induces both skin and lung fibrosis. The bleomycin model, by either subcutaneous or intratracheal injection, achieves each of these requirements (48, 49). Multiple dosing protocols of bleomycin were tested (modified from Yamamoto et al. (50))) and it was determined that a single subcutaneous dose of bleomycin 0.2 mg per 6- to 10-week-old B6 female mouse induced skin changes by 3 weeks post-injection similar to human SSc. Histologic changes included dermal thickening, loss of dermal white adipose tissue (DWAT) and loss of CD34⁺ cells (51) (FIGS. 11A-11C), along with increased dermal collagen over time (FIG. 11D). These findings concur with prior literature that B6 mice develop fibrosis by 3 weeks (49) and that a single dose of bleomycin can model pulmonary fibrosis (52). For lung fibrosis, bleomycin sulfate 0.016 mg was administered intratracheally as a single dose as previously described (53).

To characterize the dynamics of EGFR ligand expression in bleomycin-induced skin and lung fibrosis, a time course of gene expression in each tissue was performed. One week after bleomycin injection into the skin, elevated expression of the high-affinity EGFR ligand transforming growth factor alpha (Tgfa) was observed in fibrotic compared to phosphate buffered saline (PBS)-treated control skin (FIG. 11E), which aligns with previous reports that Tgfa-deficient mice are protected from developing lung fibrosis (54). Three weeks after bleomycin injection, increased epiregulin expression was observed, which coincided temporally with elevated expression of interferon signature genes by DC (FIG. 11F). These observations suggest that the initial development of fibrosis is influenced by Tgfa, but interferon-activated EREG⁺ DC are likely important later in the chronic phase, represented by three weeks in mice. The expression of epiregulin was similarly measured in mouse lungs after intratracheal administration of bleomycin and elevated epiregulin was found 1-2 weeks after bleomycin exposure (FIG. 11G). Therefore, in mice exposed to bleomycin, epiregulin has defined expression patterns during both skin and lung fibrosis, rendering the model useful for understanding epiregulin-EGFR signaling in relation to human disease.

### Example 12. Epiregulin Inhibition Alleviates Mouse and Human Skin Fibrosis

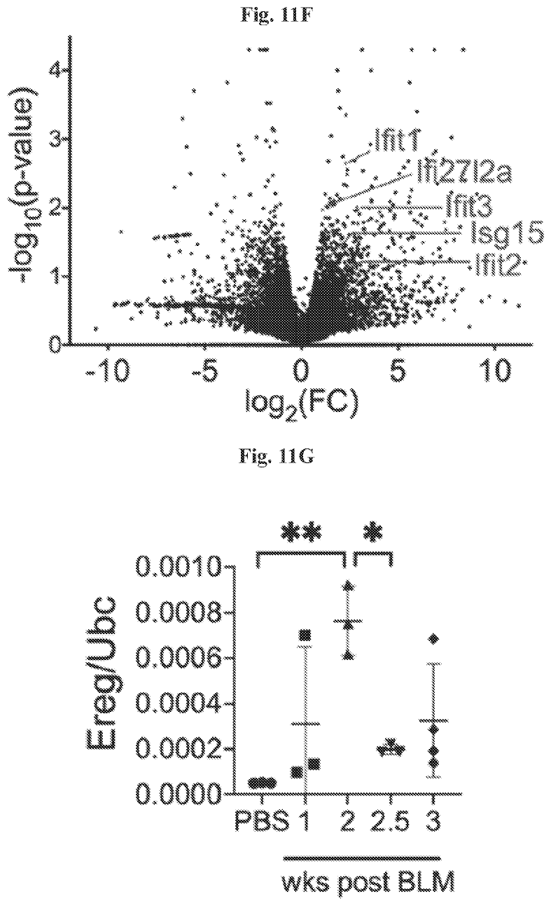

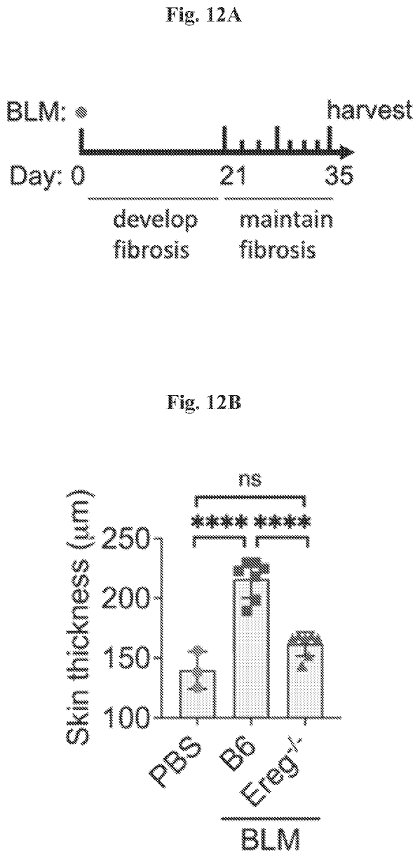

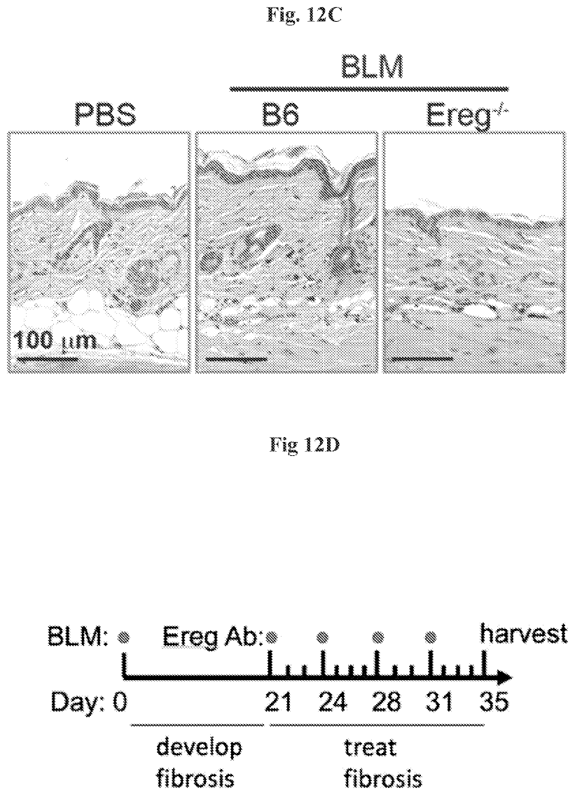

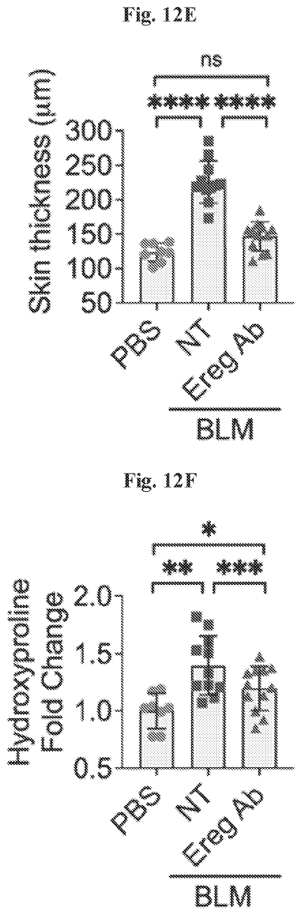

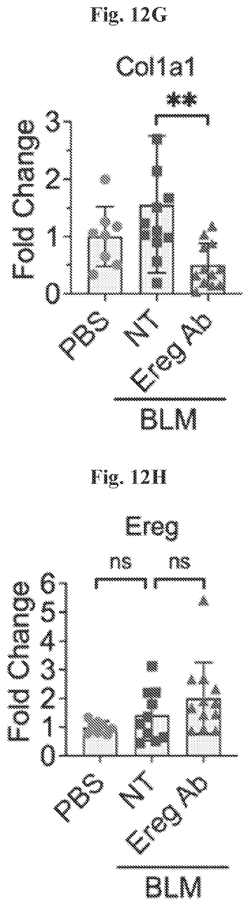

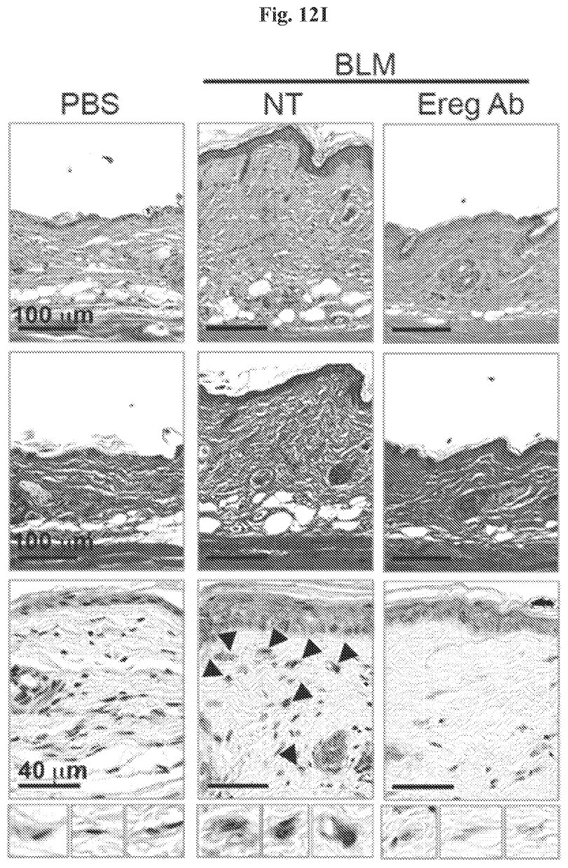

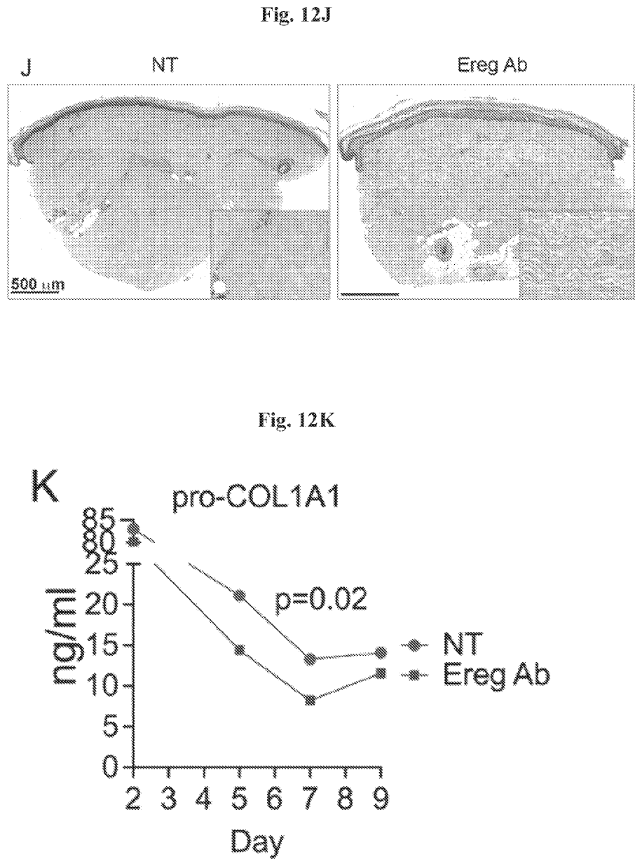

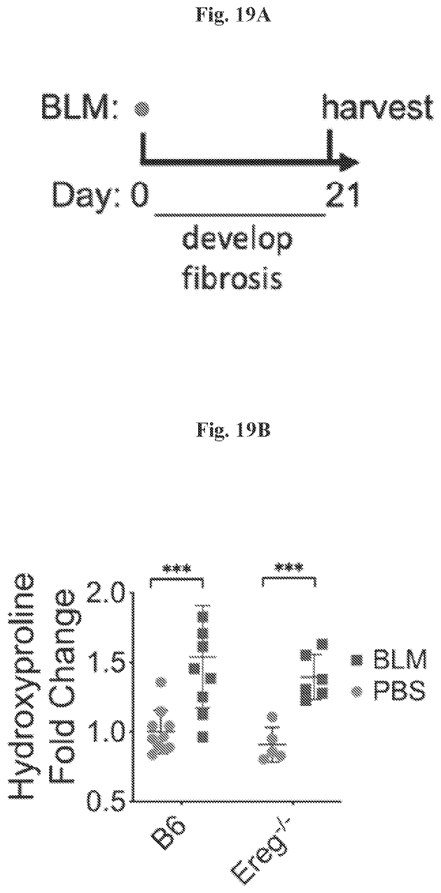

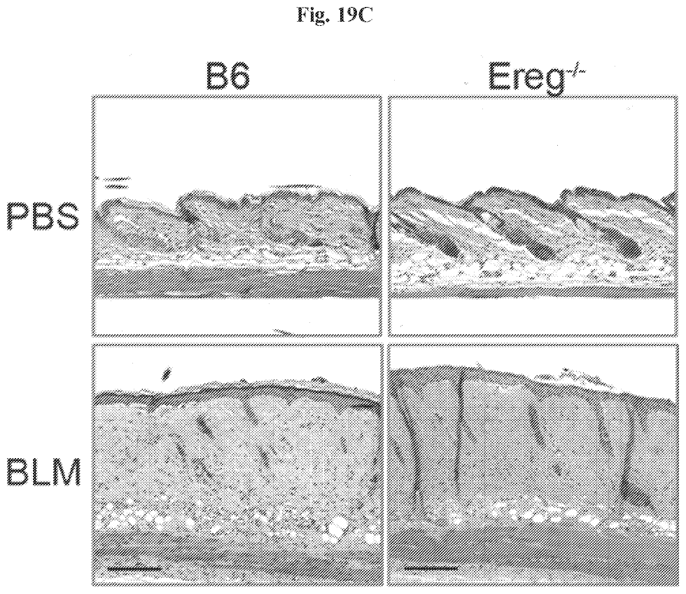

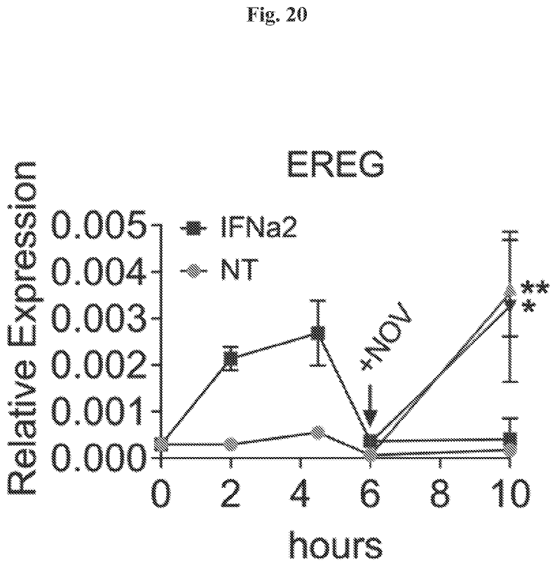

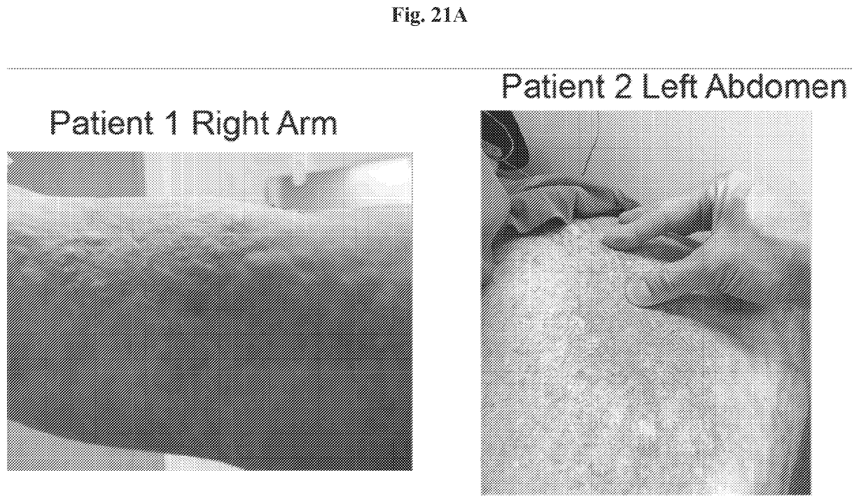

Because elevated epiregulin expression in fibrotic murine skin was delayed until 3 weeks, it was hypothesized that epiregulin would be dispensable for the development of fibrosis, but important during the chronic phase. It takes B6 mice 3 weeks to develop skin fibrosis in response to bleomycin (49). Therefore, to assess at what time epiregulin is required for development versus maintenance of skin fibrosis, the response of epiregulin-deficient mice to bleomycin was tested 3 and 5 weeks after exposure. At 5 weeks after bleomycin injection, Ereg^{-/-} mice showed decreased dermal skin thickness compared to wild type mice, suggesting that epiregulin supports the persistence of fibrosis (FIGS. 12A-12C). Decreased skin fibrosis in Ereg^{-/-} mice was not due to any defect in fibrosis development because at 3 weeks, Ereg^{-/-} mice showed dermal thickening and elevated collagen similar to their wild type counterparts (FIGS. 19A-19C). Together, these findings show that epiregulin is required for the persistence of fibrosis as opposed to fibrosis establishment.

To specifically assess the temporal role of epiregulin in the bleomycin model of skin fibrosis with a method that could be translated to patient care, epiregulin was inhibited by twice weekly subcutaneous injection of a neutralizing antibody starting 3 weeks after bleomycin injection (corresponding to its peak of expression in FIG. 11E) and diagramed in FIG. 12D. The antibody was injected at a distant location (dorsal neck) from the bleomycin (lower back). In wild type mice, two weeks of treatment with the epiregulin antibody (Ereg Ab) resulted in complete normalization of the dermal skin thickness, 50% reduction in collagen protein, and 190% reduction in collagen type I alpha 1 chain (COL1A1) expression to below PBS control levels (FIGS. 12E-12G). Ereg Ab treatment was also associated with decreased pEGFR staining in dermal cells, supporting its inhibition of EGFR activation by EREG DC (FIG. 12I, bottom row). The Ereg Ab did not alter expression of epiregulin in the skin, suggesting it does not deplete EREG DC (FIG. 12H). Thus, after skin fibrosis has been established, inhibition of epiregulin reverses dermal thickening and collagen content likely through decreased collagen expression.

To investigate whether epiregulin inhibition could be translated to treat human SSc patients, the epiregulin neutralizing antibody was tested on skin explants obtained from a patient with worsening diffuse cutaneous SSc. At the time of biopsy, the patient had a modified Rodnan Skin Score of 45 and positive RNA polymerase III antibody. Adjacent 4 mm punch biopsies from the right forearm were obtained and cultured for 9 days in the presence or absence of Ereg neutralizing antibody. Epiregulin inhibition resulted in improved histologic appearance of the skin with reduced collagen fiber thickness (FIG. 12J). Measurement of pro-COL1A1 in the non-treated skin explant media showed higher initial level at day 2 that reduced over the remaining 7 days (FIG. 12K). At each time point of culture, decreased pro-COL1A1 was observed in the Ereg Ab-treated sample compared to the non-treated control. Therefore, epiregulin inhibition appears to reduce fibrosis not just in mice, but human SSc skin as well.

### Example 13. Epiregulin Inhibition Improves Mouse and Human Lung Fibrosis

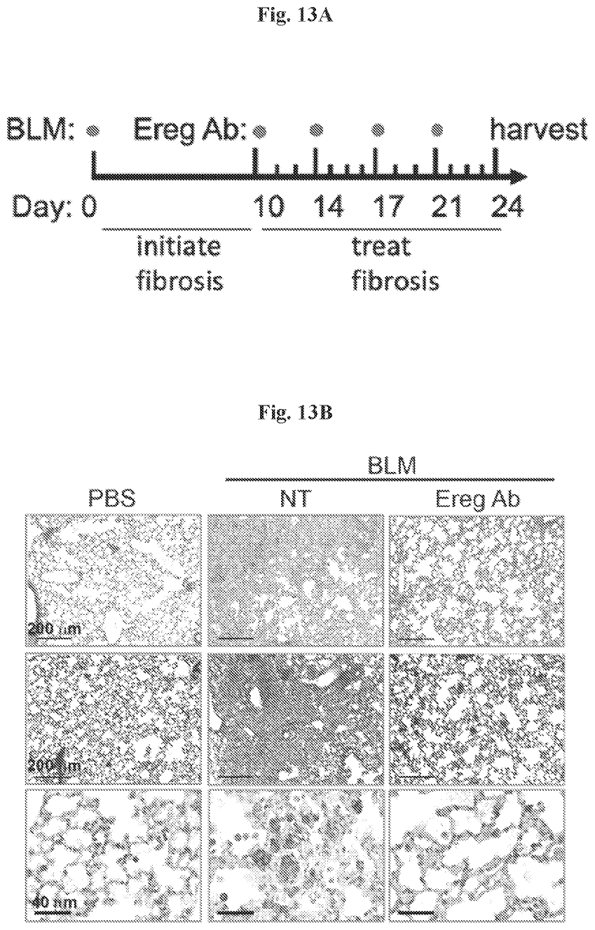

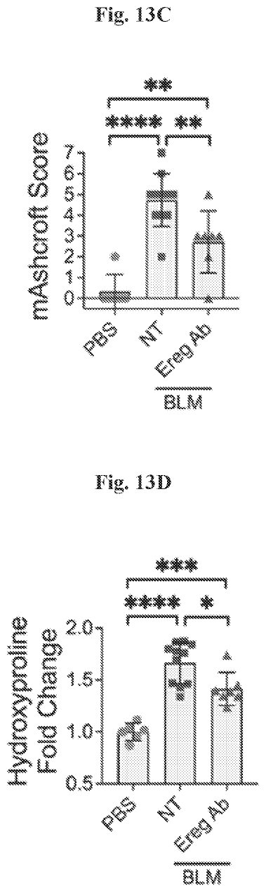

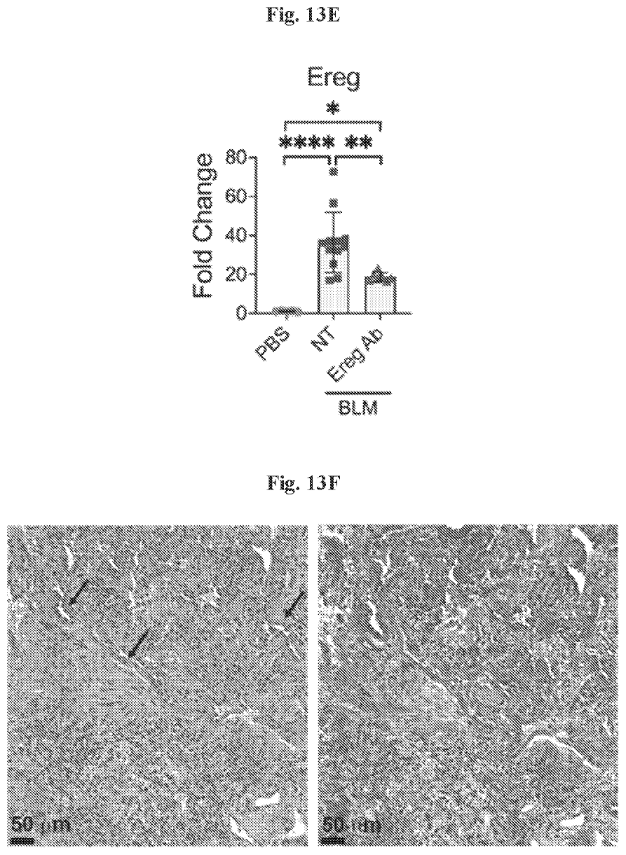

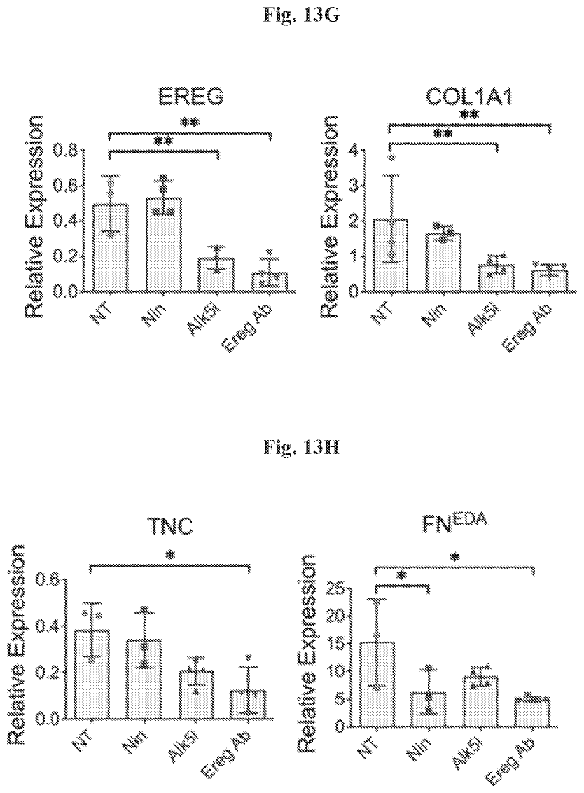

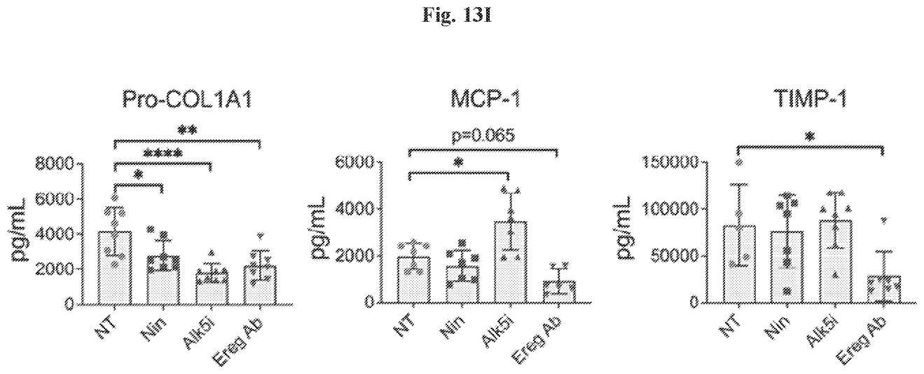

Based on the efficacy of epiregulin inhibition to reverse skin fibrosis along with its elevated expression in fibrotic mouse and human lungs, if epiregulin inhibition could also treat lung fibrosis was examined next. Epiregulin expression in lungs increased 1-2 weeks after intratracheal bleomycin exposure (FIG. 11G), so 2 weeks of antibody treatment was started on day 10 after bleomycin administration, as diagramed in FIG. 13A. In these mice, treatment with subcutaneous Ereg Ab administration prevented the development of large fibrotic masses and preserved visible alveolar septae, as shown in FIG. 13B. Clusters of pEGFR+ cells were apparent in fibrotic areas of lung of the non-treated group of bleomycin-injected mice, but absent in the PBS control and Ereg Ab-treated mice (FIG. 13B, bottom row), supporting the model of epiregulin-dependent EGFR activation. These findings corresponded with significant improvement in lung fibrosis measures with a 2-point reduction in modified Ashcroft score and 38% reduction in collagen protein (FIGS. 13C-13D). Ereg Ab treatment also decreased the expression of epiregulin from whole tissue, which may reflect lower expression of epiregulin by EREG⁺ DC or fewer number of EREG⁺ DC (FIG. 13E). Altogether, these findings show that epiregulin is also necessary for the development of lung fibrosis and is a promising therapeutic target in both skin and lung fibrosis models.

To validate the effects of epiregulin inhibition on human lung fibrosis, the impact of anti-epiregulin treatment on lung explants from a patient with IPF, which was found to show enriched expression of epiregulin and EGFR by scRNA-Seq, was investigated (FIG. 10B). Pathologic assessment confirmed the diagnosis of early IPF, as evidenced by the presence of fibroblastic foci and hyperplasia of alveolar type II epithelial cells as shown in FIG. 13F. Lung tissue was plated in 24 well plates and cultured for 10 days with media changes every two days. The impact of epiregulin antibody compared to the multikinase inhibitor nintedanib and a transforming growth factor beta type I receptor TGF-βRI/Alk5 small molecule inhibitor was assessed by measuring disease relevant endpoints in gene expression and protein production. At the gene level, reduction in epiregulin expression was observed which was similar to that seen in mouse lung by both epiregulin and TGF-βRI inhibition (FIG. 13G, left panel). These two inhibitors also significantly reduced expression of COL1A1 (FIG. 13G, right panel). Epiregulin inhibition also decreased expression of other ECM genes, including Tenascin C (TNC) and FN^{EDA} (extra domain A-containing isoform of fibronectin) (FIG. 13H, left and right panels). At the protein level, significant reduction in the levels of pro-COL1A1 by all three inhibitors (FIG. 13I, left panel) was observed. Anti-epiregulin treatment also reduced TIMP-1 levels and trended toward significance in its reduction of monocyte chemoattractant protein-1 (MCP-1) (FIG. 13I, middle and right panels). These results highlight the ability of epiregulin-blockade to impact aspects of ECM. ECM-remodeling and inflammation. Nintedanib and TGF-βRI/Alk5 inhibition also supported the reduction in some of these disease endpoints. Overall, these results support the potential for anti-epiregulin treatment as a modulator of disease relevant endpoints in IPF, and further support mouse lung bleomycin findings.

### Example 14. Interferon-EGFR-NOTCH Axis Regulates Expression of Epiregulin and ECM Genes

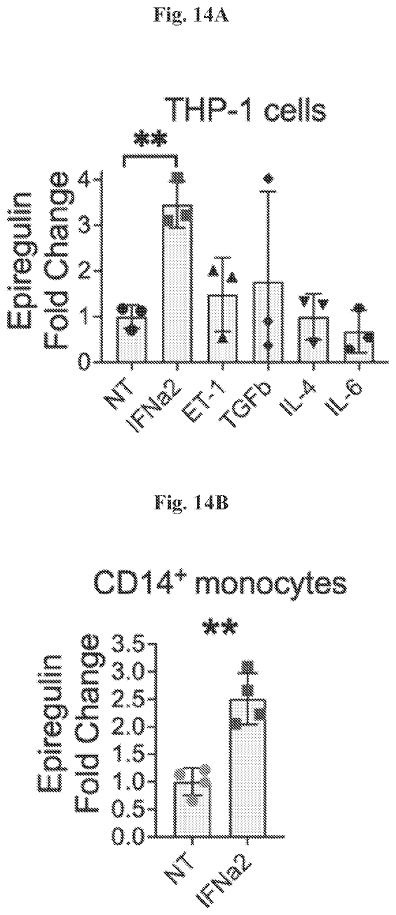

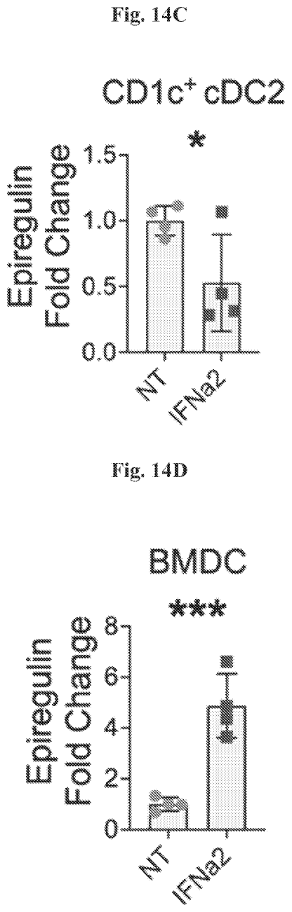

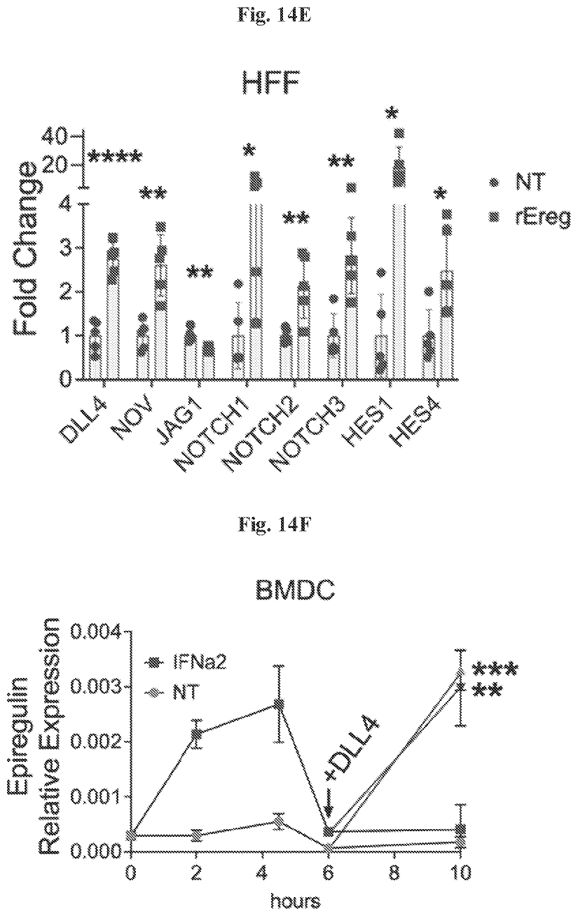

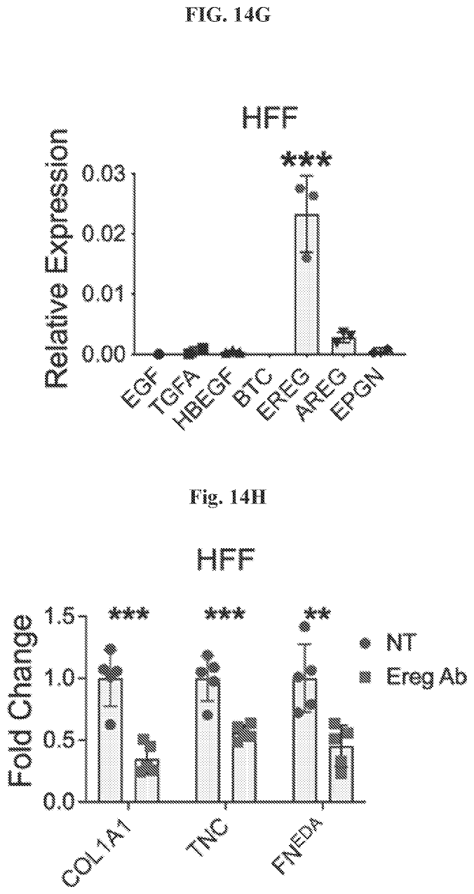

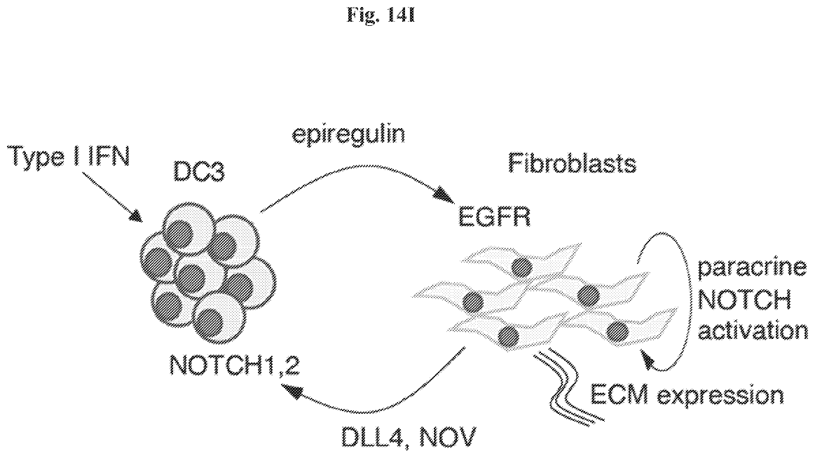

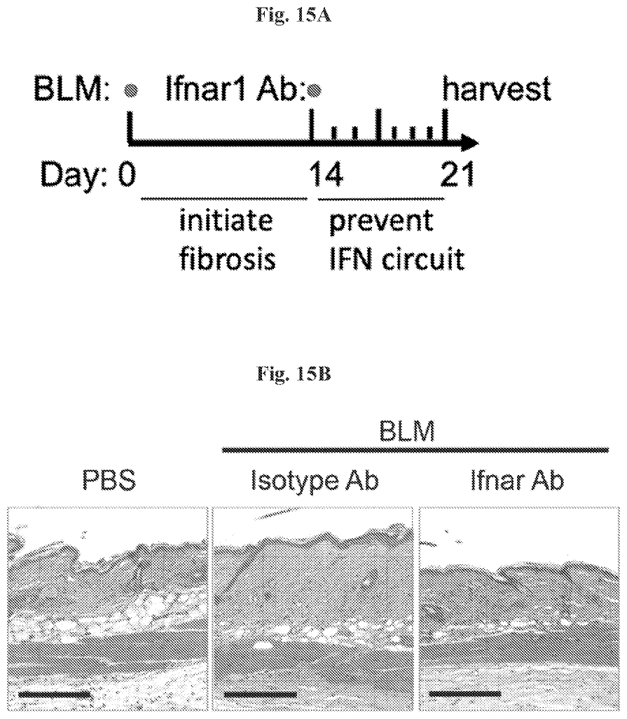

The scRNA-Seq data suggest that EREG″ DC are an inducible state of DC3. Therefore, what signals regulate epiregulin expression by human DC3 compared to circulating DC and monocytes was explored. To answer these questions. THP-1 monocytes were first screened for activating signals of epiregulin expression. Conserved regulatory elements in the first intron of the human epiregulin gene include binding sites for signal transducer and activator of transcription 1 (STAT1) complexed with signal transducer and activator of transcription 2 (STAT2) (STAT1/2). GATA Binding Protein 3 (GATA3), and FOS. STAT1/2 dimerization and activation are known to occur as a result of ligand engagement of the type I interferon receptor (55). Accordingly, higher expression of interferon stimulated genes was observed in the human scRNA-Seq data (FIG. 9F) and mouse fibrosis model (FIG. 11F). Earlier studies of epiregulin expression by cultured smooth muscle cells showed its induction by interleukin 6 (IL-6), endothelin-1, angiotensin II and α-thrombin (56, 57). Epiregulin expression in response to these cytokines was investigated and findings showed that in THP-1 monocytes, epiregulin was induced by type I interferon IFNα2 (FIG. 14A). However, epiregulin induction by endothelin-1, IL-6, IL-4, or TGFβ, the last two of which induce the transcription factors GATA3 and FOS, respectively, was not observed.

Although EREG⁺ DC showed a characteristic gene expression profile of DC3, published scRNA-Seq of peripheral blood also showed similar expression profiles between DC3 and CD14⁺ monocytes (44). Thus, epiregulin may be expressed by DC3 as well as monocytes. To determine whether monocytes, conventional dendritic cells (cDCs), or DC3 can be induced to express epiregulin, their epiregulin expression levels in response to type I interferon was tested. For monocytes and cDC, fresh peripheral blood CD14⁺ monocytes and CD1c⁺ DC were isolated. For DC3, the maturation of which depends on GM-CSF (58), human bone marrow-derived DC3 (BMDC) were generated by culturing human bone marrow with granulocyte macrophage colony-stimulating factor (GM-CSF) for seven days. IFNα2 induced epiregulin expression in both CD14⁺ monocytes and BMDC but reduced its expression in CD1c⁺ DC (FIGS. 14B-14D). These data support a model in which epiregulin expression reflects a type I interferon-inducible cellular state of DC3, which may also include CD14⁺ monocytes.

To understand how epiregulin regulates fibrotic pathways in dermal fibroblasts, the expression of growth factor ligands and receptors identified in the scRNA-Seq data was interrogated. In particular, it was noted that NOTCH receptors were commonly expressed by immune cells and fibroblasts, suggesting a potential feedback loop. Upon incubation of confluent human foreskin fibroblasts (HFFs) with recombinant epiregulin, increased expression of NOTCH ligands nephroblastoma overexpressed (NOV) and delta-like ligand 4 (DLL4), along with their respective receptors NOTCH1 and NOTCH2, was observed (FIG. 14E). While NOTCH3 expression was also increased, expression of its ligand jagged I (JAG1) was reduced. Autocrine signaling by NOTCH is tightly regulated through cis-inhibition, in which ligands inhibit NOTCH receptors on the same cell (59). In epiregulin-treated HFFs, the expression of NOTCH ligands and receptors was accompanied by increased expression of NOTCH target genes hes family basic helix-loop-helix (bHLH) transcription factor 1 (HES1) and hes family bHLH transcription factor 4 (HES4), demonstrating pathway activation in response to epiregulin. Thus, in dermal fibroblasts, epiregulin induces expression of a specific subset of NOTCH ligands and receptors, which results in activation of NOTCH pathways.

As immune cells also express NOTCH receptors, whether NOTCH ligands can signal back to EREG⁺ DC was also tested. In response to IFNa2, epiregulin expression by BMDC rose, and then fell back to baseline by 6 hours, suggesting a transient state of expression followed by loss of responsiveness to this cytokine (FIG. 14F). Subsequent exposure of these cells to NOTCH ligands DLL4 and NOV restored expression of epiregulin by BMDC (FIG. 14F and Davi). Induction of epiregulin expression through NOTCH occurred to a similar level irrespective of interferon priming. Therefore, in addition to type I interferon. NOTCH ligands are additional inducers of epiregulin expression in primary DC and thereby act as a positive feedback signal from SSc fibroblasts to maintain EREG⁺ DC.

Given the ability of epiregulin to drive NOTCH activation, it was next hypothesized that epiregulin would also modulate ECM gene expression. Unlike fresh adult dermal fibroblasts, cultured HFF expressed epiregulin in an autocrine manner (FIG. 14G). This observation was taken advantage of to test if an epiregulin neutralizing antibody (Ereg Ab) could reduce ECM gene expression in cultured HFF, and it was found that Ereg Ab significantly reduced expression of COL1A1. TNC, and FN^{EDA} (FIG. 14H). Overall, these results reveal a multicellular circuit (FIG. 14I), in which type I interferon induces epiregulin expression in DC3. EREG⁺ DC in turn activate NOTCH signaling in fibroblasts, and fibroblast-derived NOTCH ligands bind NOTCH receptors on EREG⁺ DC to maintain epiregulin expression. Furthermore, epiregulin-mediated EGFR activation is necessary for expression of multiple fibrotic ECM genes.

### Example 15. Inhibition of Type I Interferon-EGFR-NOTCH Axis Prevents Fibrosis In Vivo

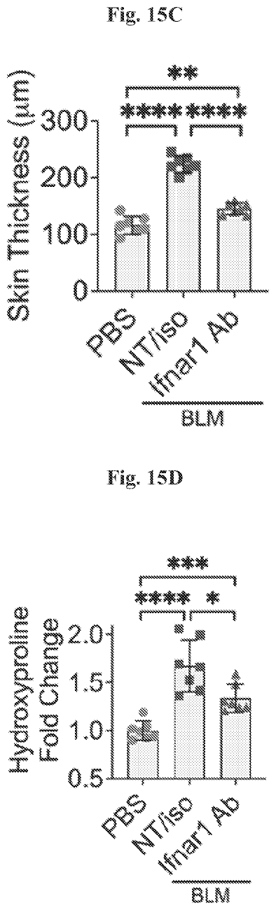

Initiation of the EGFR-NOTCH circuit by type I interferon implies that interferon inhibition prior to circuit activation during fibrosis should reduce epiregulin expression and NOTCH activation. To test if EGFR-NOTCH circuit activation depends on interferon in vivo, the bleomycin dermal skin fibrosis model was used. Two weeks after subcutaneous bleomycin injection, mice were treated intraperitoneally with an interferon receptor (interferon alpha and beta receptor subunit 1. Ifnar1) blocking antibody to inhibit activation of monocytes and DCs by type I interferon (diagramed in FIG. 15A), which it was hypothesized would prevent initiation of the signaling circuit. Indeed, compared to vehicle and isotype antibody-treated controls. Ifnar1 antibody-treated mice showed reduced skin thickness and collagen content (FIGS. 15B-15D). Furthermore, relative expression of epiregulin and the Notch target gene Hes1 were reduced in the Ifnar1-antibody treated mice (FIGS. 15E-15F). This signifies that when administered prior to epiregulin induction, interferon inhibition could reduce fibrosis along with the stimulus for epiregulin expression, and thereby prevented NOTCH activation. Thus, the interferon-EGFR-NOTCH circuit appears activated in vivo during skin fibrosis. In summary, through scRNA-Seq analysis of SSc skin and lung, an aberrantly activated DC-fibroblast signaling circuit centered on the expression of epiregulin by DC3 that is triggered by type I interferon was identified. Targeted inhibition of epiregulin reversed fibrosis in both mouse skin and lung models and patient explants, identifying epiregulin as a potentially groundbreaking therapy for patients suffering from SSc and other fibrotic diseases.

The present Examples reveal how type I interferon induces the EGFR ligand epiregulin to activate a multicellular circuit that drives the persistence of skin and lung fibrosis. EREG⁺ DC were a recently identified rare population of dendritic cells with unclear function (43). Epiregulin is one of seven cell-surface EGFR ligands, previously reported to protect the gastrointestinal tract from dextran sulfate sodium colitis (60)) and to signal with betacellulin and amphiregulin to induce maturation of the ovarian follicle (61). This work shows that epiregulin, the defining ligand of EREG DC, drives a multicellular circuit to activate NOTCH signaling and ECM expression in human fibroblasts. In patients, the circulating levels of type I interferon correlate with severity of fibrosis in the skin and lung in SSc (35, 62). Likewise, it show herein that the circuit driving epiregulin is abrogated by blocking type I interferon signaling. Thus, the present findings provide a compelling mechanism to explain these observations in SSc disease pathogenesis, whereby chronically elevated type I interferon in SSc patients drives epiregulin expression to induce EGFR and NOTCH activation and excess ECM production. Consequentially, targeted inhibition of epiregulin is able to interrupt this circuit and reverse fibrotic disease.

EGFR and NOTCH are both developmental morphogens that have been observed to interact in progenitor cells, but the association between their signaling pathways has not previously been appreciated in fibrosis. Earlier studies identified cross-talk between EGFR and NOTCH during development of the retina (63) and vulva (64), and in regulation of neural stem cell fate (65). NOTCH3 was recently identified to drive fibroblast-mediated inflammatory arthritis (66), although it is not known if crosstalk occurs with EGFR in this context. The present Examples indicate that epiregulin induces NOTCH3 expression in fibroblasts, which may regulate fibroblast NOTCH activation. Further. EGFR and NOTCH are coopted to signal between immune and mesenchymal cells in human fibrotic disease (as diagramed in FIG. 14I) and EGFR activation marks those fibroblasts with excessive ECM production in SSc.

Earlier studies showed that global EGFR inhibition could prevent the development of skin, liver, and kidney fibrosis (8-10). Taken alone, these observations suggested that EGFR inhibitors could be therapeutically effective in patients. However, clinical studies of broadly acting tyrosine kinase inhibitors for treatment of skin and lung fibrosis have been marred by the development of serious adverse events (67, 68). The findings disclosed herein indicate that targeted inhibition of the EGFR ligand epiregulin reverses collagen and other ECM genes to homeostatic levels in fibrosis animal models and tissue explants. Epiregulin thereby provides a promising therapeutic target for clinical development to treat multiple fibrotic diseases Below are the methods used in the Examples described above.

Study Patients. Skin biopsies from five patients with diffuse cutaneous SSc and five healthy controls were analyzed with single-cell RNA sequencing. Adult patients with diffuse scleroderma (systemic sclerosis) diagnosed by American College of Rheumatology criteria (69) and healthy controls were recruited for study approved by the Yale Human Investigation Committee (HIC #1511016816). The clinical diagnoses of scleroderma was confirmed by histopathology of the skin in all patients. Women and minorities were not excluded from this study based on sex/gender, race, or ethnicity. The patient's clinical data, including age, sex, age of onset of disease, duration of disease, family history of autoimmune disease, and current and previous treatments, were reviewed by Dr. Odell. Dr. Odell was the only member with access to de-identified patient data. Exclusion criteria included evidence of overlapping autoimmune disease, chronic bloodborne infections including HIV and hepatitis B and C, and inability to provide informed consent. Healthy controls were excluded if they had personal or family history of autoimmune disease.

Single-cell tissue preparation and RNA sequencing. After anesthetizing a 1-2 cm area of skin with 1% lidocaine hydrochloride with epinephrine 1:100,000, two adjacent punch biopsies measuring 6 mm and 3 mm were performed. The 3 mm biopsy was fixed in 10% neutral buffered formalin and the 6 mm biopsy was immediately processed for single cell RNA library preparation. The entire 6 mm specimen was first incubated in RPMI 1640 medium (Gibco) containing 5% fetal bovine serum (5% FBS/RPMI) and 10 mg/ml Dispase II (Sigma D4693-1G) for 45 minutes at 37° C., shaking at 200-250 rpm. The 6 mm sample was then removed from the media and minced with sterile iris scissors, followed by digestion with Liberase™ (Sigma) 0.5 mg/ml and DNase I 30 Units/ml in 5% FBS/RPMI for 45 minutes at 37° C., shaking at 200-250 rpm. The resulting single cell suspension was then filtered through a 70 μm nylon membrane and washed. Live cells were sorted on a FACSAria at the Yale Flow Core and their final concentration and viability quantified with Trypan blue on a hemacytomer. The cells were pelleted and suspended in phosphate buffered saline containing 0.04% bovine serum albumin between 500-1000 cells/μl. 3000-6000 cells with greater than 80% viability were submitted to the Yale DNA Sequencing facility for generation of single-cell cDNA libraries using the Chromium Single Cell Controller (10× Genomics).

Single-cell analysis. Each cDNA library generated from a 6 mm skin sample was sequenced paired-end on 1 lane with 75 base-pair read length using the Illumina HiSeq 2500 System generating at least 75,000 reads per cell. The 10× genomics Cell Ranger pipeline was then used to align the reads, perform clustering and gene expression analysis, and aggregate the samples with normalized read counts, t-Distributed Stochastic Neighbor Embedding (tSNE) plots used 10 principal components. The raw matrices from Cell Ranger were also processed with Seurat version 3 R toolkit for single cell genomics (70, 71) to filter low quality samples, followed by data normalization (log normalized using the default scaling factor of 10000), scaling. PCA analysis, UMAP clustering, and generation of violin plots, and subsequently analyzed with CellphoneDB v2.0 (39, 40) for receptor-ligand enrichment. All 10× experiments were completed with the same 3′ chemistry and high-throughput sequencer to avoid batch effects. Raw matrices from skin and lung scRNA-Seq were downloaded from the NCBI Gene Expression Omnibus (GSE138669, GSE122960, GSE128169, GSE132771 and GSE163973) and analyzed as above using the Seurat toolkit followed by CellphoneDB. To best match the age and sex of the SSc samples in Reyfman et al (41), donor numbers 1, 3, 4, and 7 were used for healthy control data. Heatmaps of gene expression were generated from the cell clusters in the 10× Loupe Browser v5 with MORPHEUS software (software.broadinstitute.org/morpheus).

Immunohistochemistry of skin and lung sections. From patient skin. 3 mm skin biopsy samples were fixed in 10% neutral buffered formalin for 24 hours prior to embedding in paraffin. Samples were processed at the Yale Pathology Tissue Services. For immunohistochemistry analysis, 5 μm sections were cut, and slides were deparaffinized and rehydrated to distilled water. They were then placed in TBS with tween. This is the same solution that is used in subsequent washing steps. Endogenous peroxidase was blocked using 3% hydrogen peroxide and then rinsed. The slides were then treated with Proteinase K for 7 min and rinsed. For pEGFR and vimentin co-labelling, heat-induced epitope retrieval was utilized. The slides were incubated with primary antibody, rinsed and the antibodies detected with HRP-conjugated secondary antibody. DAB was used to identify the reaction, then the slides were washed and counterstained in hematoxylin, dehydrated, cleared and mounted with resinous mounting media. Quantification of epiregulin and EGFR positive cells was completed in blinded fashion by scoring the number of positive cells in 10 high powered fields.

Primary Antibodies and dilutions: Rabbit anti-human/mouse EGFR (phospho Y1068) antibody (clone EP774Y, Abcam ab40815) 1/400 dilution with human tissue, 1/800 dilution with mouse tissue; Rabbit IgG isotype control (clone EPR25A, Abcam ab172730) 1/400; Anti-human epiregulin antibody (R&D Biosciences AF1195) 5 μg/mL; Anti-mouse/human epiregulin antibody (clone 189611, R&D Biosciences MAB1068); Anti-mouse IFNAR-1 antibody (clone MAR1-5A3 from Bio X Cell)

Animals. Wild type C57BL/6 were purchased from Charles River Laboratories. Mgl2^{DTReGFPpANeo} (Mgl2-DTR-GFP) mice were kindly provided by Akiko Iwasaki (Yale University). All mice were maintained at the Yale University School of Medicine Animal Resources Center. Mouse experiments were conducted under a protocol approved by the Yale University Institutional Animal Care and Use Committee and in accordance with AAALAC guidelines.

Bleomycin mouse models of fibrosis. Mice were anesthetized using an isoflurane precision vaporizer. To induce skin fibrosis, mice were laid on their abdomen, then a 2×2 cm area of fur removed with electric clippers, and then injected subcutaneously with 0.2 mg bleomycin sulfate (Sigma B8416) diluted in 0.2 ml sterile PBS (10 mg/kg) or 0.2 ml PBS vehicle control using 30-gauge needle. To induce lung fibrosis, mice were suspended vertically by their incisors and administered bleomycin sulfate 1.25 U/kg intratracheally in 60 μL PBS as previously described (53). From lung specimens, the right three lobes were used for hydroxyproline quantification, the left upper lobe for histology, and the left lower lobe for gene expression. Modified Ashcroft score (72) of the lung was calculated in a blinded manner. All experiments have wild type (B6) controls to account for variation in potency of bleomycin lots to induce fibrosis. Fibrosis was measured on day 21 after bleomycin injection unless otherwise noted. Epiregulin neutralizing antibody (clone 189611, R&D Systems MAB1068) 10 mg/kg diluted in 100 μl PBS was given subcutaneously twice weekly on the dorsal neck of anesthetized mice. To block type I interferon signaling, 1.67 mg IFNAR-1 antibody (clone MAR1-5A3 from Bio X Cell) diluted in 0.5 mL PBS was administered intraperitoneally as a single dose 2 weeks after bleomycin injection. For bulk RNA sequencing of skin dendritic cells. Mgl2^{DTReGFPPANeo} mice were injected subcutaneously with bleomycin as above, but not injected with diphtheria toxin. 3 weeks later, the affected skin was harvested and digested with Liberase™ (Sigma) 0.5 mg/ml and DNase I 30 Units/ml in 5% FBS/RPMI for 1 hour at 37° C., shaking at 200-250 rpm. The resulting single cell suspension was then filtered through a 70 μm nylon membrane and washed. Dendritic cells were sorted from macrophages by gating on the CD64-population of live GFP⁺ cells followed by bulk RNA sequencing, and analysis with the Tuxedo suite of applications.

Patient skin and lung explant cultures. For skin explant culture, two adjacent 4 mm punch biopsies were obtained from the right arm of patient SSc6. They were immediately placed in skin media of DMEM containing 4.5 g/L D-glucose. L-glutamine, 0.1% FBS, 100 U/ml penicillin-streptomycin and 2.5 mg/L amphotericin B. Excess subcutaneous fat was carefully removed with iris scissors, then each biopsy specimen was lightly floated epidermis side up exposing it to air in the center of a 12-well tissue culture plate containing 1 ml of skin media alone or with addition of anti-mouse/human epiregulin antibody 2.5 μg/ml, then incubated at 37° C., in 5% CO₂ humidified incubator. The media was changed after 2 hours, then on days 2, 5, and 7. Used skin media was stored at −20° C., prior to protein measurements. Pro-COL1A1 was measured using ELISA kit per manufacturer instructions (Abcam, ab210966).

For lung explants, human IPF lung (44 year-old hispanic male) was obtained from the National Disease Research Interchange (NDRI), kept cold, and received <24 hours after cross-clamp. A diagnosis of IPF, of 2 years duration, was confirmed by NDRI. While kept cold, tissue was first cut into rough strips, then cut into very thin strips. From there, the tissue was cut into small pieces suitable for culture (roughly 50-100 mg in size). Fragments were cultured in 24 well plates in Dulbecco's Modified Eagle's Medium; F-12 Ham's Nutrient Mixture (DMEM; F-12, Gibco 11039-021) without phenol red, containing L-glutamine, 15 mM HEPES, sodium bicarbonate, penicillin, streptomycin and amphotericin antibiotic-antimycotic solution (Gibco), 50 μg/ml Gentamicin and 1× Insulin-Transferrin-Selenium-Ethanolamine liquid supplement (Sigma). Media changes were performed every 2 days, for a total of 10 days, with replacement of described pharmacologies occurring each media change. After 10 days, culture supernatants were collected, pre-cleared at 4000× g (4° C. 10 mins), transferred to a clean 96 well polypropylene plate and stored at −80° C., until protein endpoints measured. Measured secreted proteins include: human MCP-1 V-Plex (MesoScale Discovery, K151NND-1), human TIMP-1 (MesoScale Discovery, K151JFC-1) and human pro-collagen 1α1 DuoSet Elisa (R&D systems, DY6220-05). All proteins were assayed and analyzed in accordance with their respective assay product datasheets. After 10 days, lung fragments were harvested and dissociated in 350 μl of RLT buffer containing β-mercaptoethanol using a TissueLyser II (Qiagen) and mRNA isolated with the Rneasy Fibrous mini kit (QiaGen, 74704). Secreted protein results represent 8 individual fragments and gene expression represents 4 individual fragments per treatment group.

Hydroxyproline Analysis. After euthanasia, the shaved skin or lung was stored at −70° C., prior to processing. From the skin, a 2 mm punch biopsy (Accu-Punch) was obtained from the affected area for hydroxyproline quantitation using a Hydroxyproline Assay Kit (Sigma MAK008). Briefly, the 2 mm skin specimen or 3 right lung lobes were boiled in 100 μl or 500 μl, respectively, of 37% hydrochloric acid for 3 hours at 120° C. The sample was then centrifuged for 1 minute at 16,000×g to pellet any remaining hair and debris. From the supernatant, 3 μl was transferred to a fresh microcentrifuge tube and allowed to air dry with the top open at 60° C., for approximately 25 minutes. The dried pellet was suspended in 100 μl Chloramine T/Oxidation Buffer mixture for 5-10 minutes, followed by addition of 100 μl diluted 4-(Dimethylamino)benzaldehyde and incubated for 90 minutes at 60° C. The 550 nm absorbance was measured with either BioRad iMark or BioTek Synergy HTX microplate reader.

Cell Lines. THP-1 monocytes and human foreskin fibroblasts were purchased from ATCC (TIB-202 and SCRC-1041), CD14⁺ monocytes and CD1c⁺ DC precursors were isolated from freshly obtained peripheral blood from healthy volunteers using the Human CD14 Positive Selection Kit II from STEMCELL Technologies and CD1c⁺ Human Dendritic Cell Isolation Kit from Miltenyl Biotec per manufacturers' protocols. Human BMDC were generated by incubating bone marrow from MISTRG6 humanized mice (73, 74) with human GM-CSF (R&D Biosciences 215-GM) 100 ng/ml for 7 days.

Monocyte and dendritic cell gene expression. Monocytes and dendritic cells were incubated in Gibco Roswell Park Memorial Institute (RPMI) 1640 Medium containing 10% fetal bovine serum (FBS) at 37° C., in 5% CO₂ humidified incubator. They were incubated with cytokines for four hours unless otherwise indicated prior to RNA isolation and cDNA synthesis using the following concentrations: IFNa2 1000 U/mL (Biolegend 592704). TGF-β1 0.64 ng/ml (R&D Biosciences 7754-BH), endothelin-1 100 ng/ml (Abcam ab158332), IL-4 25 ng/ml (R&D Biosciences 6507-IL), and IL-6 100 ng/ml (R&D Biosciences 206-IL). To test the effects of NOTCH ligands, monocytes were incubated in media alone or IFNa2 1000 U/mL for 6 hours at 37° C. During the last 45 minutes, recombinant NOTCH ligands DLL4 (R&D Biosciences 1506-D4) and NOV (R&D Biosciences 1640-NV) each 10 μg/mL were adhered to the bottom of 48 well plates at 37° C., for 45 minutes as described (75). The monocytes were pelleted and suspended in fresh media, then transferred to the wells containing NOTCH ligands or media alone.

Fibroblast gene expression. Human foreskin fibroblasts (HFF) were seeded in Dulbecco's Modified Eagle Medium (DMEM) containing 1% FBS overnight at 37° C., in 5% CO₂ humidified incubator. The following day, the media was removed, and fresh media was added. To test the effects of recombinant human epiregulin (R&D Biosciences 1195-EP) on NOTCH signaling, 1 μg/ml was added to confluent HFF in media supplemented with ascorbic acid 50 μg/mL overnight prior to RNA extraction and cDNA synthesis. For EGFR ligand expression, sub-confluent HFF were incubated for 48 hours in media alone. For EGFR inhibition, sub-confluent HFF were incubated in media alone or with anti-human epiregulin neutralizing antibody (R&D Biosciences AF1195) 5 μg/mL for 24 hours prior to RNA isolation and qPCR analysis.

RNA preparation and Quantitative PCR (qPCR). RNA was extracted from tissue and cells using RNeasy Mini Plus Kit (Qiagen). Skin and lung tissue were disrupted and homogenized with Qiagen TissueRuptor II in RLT Plus buffer containing β-mercaptoethanol 1:100 dilution and Reagent DX (Qiagen) 1:200 dilution until no remaining intact tissue was visible, approximately 30-60 seconds, cDNAs were developed using Maxima H Minus Reverse Transcriptase 10 U/μl supplemented with 0.5 mM dNTPs and 25 ng/μl Oligo d (T) 20 (SEQ ID NO: 73). Primers were purchased from Sigma-Aldrich and sequences are listed below for human qPCR sequences (Table 5) and mouse qPCR sequences (Table 6). Relative quantification of gene expression using SYBR Green was measured with CFX384 or CFX96 Touch Real-Time PCR Detection System (Bio-Rad). Gene expression was normalized to the housekeeping gene UBC (76) or PPIA (lung explants) and relative expression was calculated using the 2^{-ΔΔCt} method (77).

TABLE 5
Human qPCR sequences

| | | SEQ | | SEQ |
|---|---|---|---|---|
| Gene | Forward Primer Sequence | ID | Reverse Primer Sequence | ID |
| UBC | CGTCACTTGACAATGCAG | 11 | TGTTTTCCAGCAAAGATCAG | 12 |
| COL1A1 | GCTATGATGAGAAATCAACCG | 13 | TCATCTCCATTCTTTCCAGG | 14 |
| FN1^{EDA} | TAAAGGACTGGCATTCACTGA | 15 | GTGCAAGGCAACCACACTGA | 16 |
| (78) | | | | |
| TNC | GTGGGATCCTCTAGACATTG | 17 | GTGATCTCTCCCTCATCTTC | 18 |
| EGF | CAGTTGATCCAGTAGAAAGG | 19 | TGTACTGAATCCAAAACAGC | 20 |
| TGFA | AGAAACAGTGGTCTGAAGAG | 21 | ATTACAGGCCAAGTAGGAAG | 22 |
| BTC | CAGAAGTCCTGAAACTAATGG | 23 | CAATGTAGCCTTCATCACAG | 24 |
| HBEGF | GCTTATATACCTATGACCACA | 25 | GTACCTAAACATGAGAAGCC | 26 |
| EREG | GTTCAGACAGAAGACAATCC | 27 | GACTCATGTCCACCAGATAG | 28 |
| AREG | AAAGAAAGAAAAAGGGAGGC | 29 | CATTTGCATGTTACTGCTTC | 30 |
| EPGN | GCAAGCTGACAACATAGAAG | 31 | GCAGTAACTGTTATGATCTTC | 32 |
| DLL4 | GTTACACAGTGAAAAGCCAG | 33 | CTCTCCTCTGATATCAAACAC | 34 |
| NOV | TAGAAGTCTCTGACTCAAGTG | 35 | CCTTTCTTATCTGTTGGCTG | 36 |
| JAG1 | ACTACTACTATGGCTTTGGC | 37 | ATAGCTCTGTTACATTCGGG | 38 |
| NOTCH1 | AAGATATGCAGAACAACAGG | 39 | TCCATATGATCCGTGATGTC | 40 |
| NOTCH2 | GATGAATGATGGTACTACACC | 41 | CAGATTTTCCATGGTCATCC | 42 |
| NOTCH3 | CTACAATGGTGATAACTGTGA | 43 | CAGTCATCCTCATTAATCTCG | 44 |
| NOTCH4 | GTCCCCCAGGTTTCATAG | 45 | ACTCATCCACATCTTCGG | 46 |
| HES1 | GCCTATTATGGAGAAAAGAC | 47 | CTATCTTTCTTCAGAGCATCC | 48 |
| HES4 | AAAACCCTCATCCTGGAC | 49 | GTGTCTCACGGTCATCTC | 50 |

TABLE 6
Mouse qPCR sequences

| | | SEQ | | SEQ |
|---|---|---|---|---|
| | | ID | | ID |
| Gene | Forward Primer Sequence | NO. | Reverse Primer Sequence | NO. |
| Ubc | GAGACGATGCAGATCTTTG | 51 | ATGTTGTAGTCTGACAGGG | 52 |
| Col1a1 | GCTCCTCTTAGGGGCCACT | 53 | CCACGTCTCACCATTGGGG | 54 |
| (79) | | | | |
| Fn1^{EDA} | AGATGCAGCAGATCCGCAT | 55 | GTTCTTGCCCATCAGCACC | 56 |
| (78) | | | | |
| Tnc | CAAAGAGACCTTCATCACAG | 57 | GGCTCTTTGGAACATCTATC | 58 |
| Egf | GTACTCTTGGGTGTGAAAAC | 59 | CAAGTTCGTGACATTGTTTC | 60 |
| Tgfa | CTCTGAGACAGTGGTCTG | 61 | GTCCCAAGATGCCTTTTC | 62 |
| Btc | AGACCAATATTGCTTAACGG | 63 | AACAAGCAACACACAGTTAC | 64 |
| Hbegf | GAGCTATAGGAACCTTCAGAG | 65 | ACTGGAAATGAATGAAGACG | 66 |
| Ereg | AATTCTGCAGATGTGAAGTG | 67 | TATTCTTTGCTCAAGGGTTG | 68 |
| Areg | GACATGCAATTGTCATCAAG | 69 | GACAAAGATAGTGACAGCTAC | 70 |
| Epgn | ATAGAAGAACCTGTAGCTCTG | 71 | CCGTATAACCAGTAAAGCATC | 72 |

### REFERENCES

1. Gieseck R L, 3rd, Wilson M S, and Wynn T A. Type 2 immunity in tissue repair and fibrosis. Nat Rev Immunol. 2017.
2. Distler O, Highland K B, Gahlemann M, Azuma A, Fischer A, Mayes M D, et al. Nintedanib for Systemic Sclerosis-Associated Interstitial Lung Disease. N Engl J Med. 2019.
3. Khanna D, Lin C J F, Furst D E, Goldin J, Kim G, Kuwana M, et al. Tocilizumab in systemic sclerosis: a randomised, double-blind, placebo-controlled, phase 3 trial. Lancet Respir Med. 2020; 8 (10): 963-74.
4. Olson L E, and Soriano P. Increased PDGFRalpha activation disrupts connective tissue development and drives systemic fibrosis. Dev Cell. 2009; 16 (2): 303-13.
5. Chakraborty D, Zhu H, Jungel A, Summa L, Li Y N, Matei A E, et al. Fibroblast growth factor receptor 3 activates a network of profibrotic signaling pathways to promote fibrosis in systemic sclerosis. Sci Transl Med. 2020; 12 (563).
6. Wilson K J, Gilmore J L, Foley J, Lemmon M A, and Riese D J, 2nd. Functional selectivity of EGF family peptide growth factors: implications for cancer. Pharmacol Ther. 2009; 122 (1): 1-8.
7. Lofgren S, Hinchcliff M, Carns M, Wood T, Aren K, Arroyo E, et al. Integrated, multicohort analysis of systemic sclerosis identifies robust transcriptional signature of disease severity. JCI Insight. 2016; 1 (21): e89073.
8. Fuchs B C, Hoshida Y, Fujii T, Wei L, Yamada S, Lauwers G Y, et al. Epidermal growth factor receptor inhibition attenuates liver fibrosis and development of hepatocellular carcinoma. Hepatology. 2014: 59 (4): 1577-90.
9 Morin F, Kavian N, Marut W, Chereau C, Cerles O, Grange P, et al. Inhibition of EGFR Tyrosine Kinase by Erlotinib Prevents Sclerodermatous Graft-Versus-Host Disease in a Mouse Model. J Invest Dermatol. 2015: 135 (10): 2385-93.
10. Liu N, Guo J K, Pang M, Tolbert E, Ponnusamy M, Gong R, et al. Genetic or pharmacologic blockade of EGFR inhibits renal fibrosis. J Am Soc Nephrol. 2012: 23 (5): 854-67.
11. Suzuki H, Aoshiba K, Yokohori N, and Nagai A. Epidermal growth factor receptor tyrosine kinase inhibition augments a murine model of pulmonary fibrosis. Cancer Res. 2003: 63 (16): 5054-9.
12. Zhou X, Franklin R A, Adler M, Jacox J B, Bailis W, Shyer J A, et al. Circuit Design Features of a Stable Two-Cell System. Cell. 2018: 172 (4): 744-57 e17.
13. Sole-Boldo L, Raddatz G, Schutz S. Mallm J P, Rippe K, Lonsdorf A S, et al. Single-cell transcriptomes of the human skin reveal age-related loss of fibroblast priming. Commun Biol. 2020; 3 (1): 188.
14. Deng C C, Hu Y F, Zhu D H, Cheng Q, Gu J J, Feng Q L, et al. Single-cell RNA-seq reveals fibroblast heterogeneity and increased mesenchymal fibroblasts in human fibrotic skin diseases. Nat Commun. 2021: 12 (1): 3709.
15. Tabib T, Huang M, Morse N, Papazoglou A, Behera R, Jia M, et al. Myofibroblast transcriptome indicates SFRP2 (hi) fibroblast progenitors in systemic sclerosis skin. Nat Commun. 2021: 12 (1): 4384.
16. Buechler M B, Pradhan R N, Krishnamurty A T, Cox C, Calviello A K, Wang A W, et al. Cross-tissue organization of the fibroblast lineage. Nature. 2021: 593 (7860): 575-9.
17. Apostolidis S A, Stifano G, Tabib T, Rice L M, Morse C M, Kahaleh B, et al. Single Cell RNA Sequencing Identifies HSPG2 and APLNR as Markers of Endothelial Cell Injury in Systemic Sclerosis Skin. Front Immunol. 2018:9:2191.
18. Aliprantis A O, Wang J, Fathman J W, Lemaire R, Dorfman D M, Lafyatis R, et al. Transcription factor T-bet regulates skin sclerosis through its function in innate immunity and via IL-13. Proc Natl Acad Sci USA. 2007: 104 (8): 2827-30.
19. Satoh T, Nakagawa K, Sugihara F, Kuwahara R, Ashihara M, Yamane F, et al. Identification of an atypical monocyte and committed progenitor involved in fibrosis. Nature. 2017: 541 (7635): 96-101.
20. Ah Kioon M D, Tripodo C, Fernandez D, Kirou K A, Spiera R F, Crow M K, et al. Plasmacytoid dendritic cells promote systemic sclerosis with a key role for TLR8. Sci Transl Med. 2018: 10 (423).
21. Kraling B M, Maul G G, and Jimenez S A. Mononuclear cellular infiltrates in clinically involved skin from patients with systemic sclerosis of recent onset predominantly consist of monocytes/macrophages. Pathobiology. 1995: 63 (1): 48-56.
22. Duan H, Fleming J, Pritchard D K, Amon L M, Xue J. Arnett H A, et al. Combined analysis of monocyte and lymphocyte messenger RNA expression with serum protein profiles in patients with scleroderma. Arthritis Rheum. 2008: 58 (5): 1465-74.
23. Wohlfahrt T, Usherenko S, Englbrecht M, Dees C, Weber S, Beyer C, et al. Type 2 innate lymphoid cell counts are increased in patients with systemic sclerosis and correlate with the extent of fibrosis. Ann Rheum Dis. 2015.
24. Kahari V M, Sandberg M, Kalimo H, Vuorio T, and Vuorio E. Identification of fibroblasts responsible for increased collagen production in localized scleroderma by in situ hybridization. J Invest Dermatol. 1988: 90 (5): 664-70.
25. Scharffetter K, Lankat-Buttgereit B, and Krieg T. Localization of collagen mRNA in normal and scleroderma skin by in-situ hybridization. Eur J Clin Invest. 1988: 18 (1): 9-17.
26. Bondjers C, Kalen M, Hellstrom M, Scheidl S J, Abramsson A, Renner O, et al. Transcription profiling of platelet-derived growth factor-B-deficient mouse embryos identifies RGS5 as a novel marker for pericytes and vascular smooth muscle cells. Am J Pathol. 2003: 162 (3): 721-9.
27. Huang da W, Sherman B T, and Lempicki R A. Systematic and integrative analysis of large gene lists using DAVID bioinformatics resources. Nat Protoc. 2009: 4 (1): 44-57.
28. Henderson N C, Rieder F, and Wynn T A. Fibrosis: from mechanisms to medicines. Nature. 2020: 587 (7835): 555-66.
29. Der E, Suryawanshi H, Morozov P, Kustagi M, Goilav B, Ranabothu S, et al. Tubular cell and keratinocyte single-cell transcriptomics applied to lupus nephritis reveal type I IFN and fibrosis relevant pathways. Nat Immunol. 2019: 20 (7): 915-27.
30. Wei J, Fang F, Lam A P, Sargent J L, Hamburg E, Hinchcliff M E, et al. Wnt/beta-catenin signaling is hyperactivated in systemic sclerosis and induces Smad-dependent fibrotic responses in mesenchymal cells. Arthritis Rheum. 2012: 64 (8): 2734-45.
31. Ascension A M, Fuertes-Alvarez S, Ibanez-Sole O, Izeta A, and Arauzo-Bravo M J. Human Dermal Fibroblast Subpopulations Are Conserved across Single-Cell RNA Sequencing Studies. J Invest Dermatol. 2021: 141 (7): 1735-44 e35.
32. Plikus M V, Wang X, Sinha S, Forte E, Thompson S M, Herzog E L, et al. Fibroblasts: Origins, definitions, and functions in health and disease. Cell. 2021: 184 (15): 3852-72.
33. Tsukui T, Sun K H, Wetter J B, Wilson-Kanamori J R, Hazelwood L A, Henderson N C, et al. Collagen-producing lung cell atlas identifies multiple subsets with distinct localization and relevance to fibrosis. Nat Commun. 2020; 11 (1): 1920.
34. Kim H J, Li G, Gjertson D, Elashoff R, Shah S K, Ochs R, et al. Classification of parenchymal abnormality in scleroderma lung using a novel approach to denoise images collected via a multicenter study. Acad Radiol. 2008: 15 (8): 1004-16.
35. Eloranta M L, Franck-Larsson K, Lovgren T, Kalamajski S, Ronnblom A, Rubin K, et al. Type I interferon system activation and association with disease manifestations in systemic sclerosis. Ann Rheum Dis. 2010: 69 (7): 1396-402.
36. Lemmon M A, and Schlessinger J. Cell signaling by receptor tyrosine kinases. Cell. 2010: 141 (7): 1117-34.
37. Freed D M, Bessman N J, Kiyatkin A, Salazar-Cavazos E, Byrne P O, Moore J O, et al. EGFR Ligands Differentially Stabilize Receptor Dimers to Specify Signaling Kinetics. Cell. 2017: 171 (3): 683-95 e18.
38. Hu C, Leche C A, 2nd, Kiyatkin A, Yu Z, Stayrook S E, Ferguson K M, et al. Glioblastoma mutations alter EGFR dimer structure to prevent ligand bias. Nature. 2022.
39. Vento-Tormo R, Efremova M, Botting R A, Turco M Y, Vento-Tormo M, Meyer K B, et al. Single-cell reconstruction of the early maternal-fetal interface in humans. Nature. 2018: 563 (7731): 347-53.
40. Efremova M, Vento-Tormo M, Teichmann SA, and Vento-Tormo R. CellPhoneDB v2.0: Inferring cell-cell communication from combined expression of multi-subunit receptor-ligand complexes, bioRxiv. 2019:680926.
41. Reyfman P A, Walter J M, Joshi N, Anekalla K R, McQuattie-Pimentel A C, Chiu S, et al. Single-Cell Transcriptomic Analysis of Human Lung Provides Insights into the Pathobiology of Pulmonary Fibrosis. Am J Respir Crit Care Med. 2018.
42. Valenzi E, Bulik M, Tabib T, Morse C, Sembrat J, Trejo Bittar H, et al. Single-cell analysis reveals fibroblast heterogeneity and myofibroblasts in systemic sclerosis-associated interstitial lung disease. Ann Rheum Dis. 2019.
43. Travaglini K J, Nabhan A N, Penland L, Sinha R, Gillich A, Sit R V, et al. A molecular cell atlas of the human lung from single-cell RNA sequencing. Nature. 2020: 587 (7835): 619-25.
44. Villani A C, Satija R, Reynolds G, Sarkizova S, Shekhar K, Fletcher J, et al. Single-cell RNA-seq reveals new types of human blood dendritic cells, monocytes, and progenitors. Science. 2017: 356 (6335).
45. Girard M, Law J C, Edilova M I, and Watts T H. Type I interferons drive the maturation of human DC3s with a distinct costimulatory profile characterized by high GITRL. Sci Immunol. 2020; 5 (53).
46. Sahin U, Weskamp G, Kelly K, Zhou H M, Higashiyama S, Peschon J, et al. Distinct roles for ADAM10 and ADAM17 in ectodomain shedding of six EGFR ligands. J Cell Biol. 2004: 164 (5): 769-79.
47. Sargent J L, Li Z, Aliprantis A O, Greenblatt M, Lemaire R, Wu M H, et al. Identification of Optimal Mouse Models of Systemic Sclerosis by Interspecies Comparative Genomics. Arthritis Rheumatol. 2016: 68 (8): 2003-15.
48. Lescoat A, Lecureur V, and Varga J. Contribution of monocytes and macrophages to the pathogenesis of systemic sclerosis: recent insights and therapeutic implications. Curr Opin Rheumatol. 2021: 33 (6): 463-70.
49. Lakos G, Takagawa S, and Varga J. Animal models of scleroderma. Methods Mol Med. 2004:102:377-93.
50. Yamamoto T, Takagawa S, Katayama I, Yamazaki K, Hamazaki Y, Shinkai H, et al. Animal model of sclerotic skin. I: Local injections of bleomycin induce sclerotic skin mimicking scleroderma. J Invest Dermatol. 1999; 112 (4): 456-62.
51. Aiba S, Tabata N, Ohtani H, and Tagami H, CD34+ spindle-shaped cells selectively disappear from the skin lesion of scleroderma. Arch Dermatol. 1994: 130 (5): 593-7.
52. Snider G L, Hayes J A, and Korthy A L. Chronic interstitial pulmonary fibrosis produced in hamsters by endotracheal bleomycin: pathology and stereology. Am Rev Respir Dis. 1978: 117 (6): 1099-108.
53. Rangarajan S, Bone N B, Zmijewska A A, Jiang S, Park D W, Bernard K, et al. Metformin reverses established lung fibrosis in a bleomycin model. Nat Med. 2018: 24 (8): 1121-7.
54. Madtes D K, Elston A L, Hackman R C, Dunn A R, and Clark J G. Transforming growth factor-alpha deficiency reduces pulmonary fibrosis in transgenic mice. Am J Respir Cell Mol Biol. 1999: 20 (5): 924-34.
55. O'Shea J J, Schwartz D M, Villarino A V, Gadina M, McInnes I B, and Laurence A. The JAK-STAT pathway: impact on human disease and therapeutic intervention. Annu Rev Med. 2015:66:311-28.
56. Taylor D S, Cheng X, Pawlowski J E, Wallace A R, Ferrer P, and Molloy C J. Epiregulin is a potent vascular smooth muscle cell-derived mitogen induced by angiotensin II, endothelin-1, and thrombin. Proc Natl Acad Sci USA. 1999: 96 (4): 1633-8.
57. Kyotani Y, Itaya-Hironaka A, Yamauchi A, Sakuramoto-Tsuchida S, Makino M, Takasawa S, et al. Intermittent hypoxia-induced epiregulin expression by IL-6 production in human coronary artery smooth muscle cells. FEBS Open Bio. 2018: 8 (5): 868-76.
58. Bourdely P, Anselmi G, Vaivode K, Ramos R N, Missolo-Koussou Y, Hidalgo S, et al. Transcriptional and Functional Analysis of CD1c (+) Human Dendritic Cells Identifies a CD163 (+) Subset Priming CD8 (+) CD103 (+) T Cells. Immunity. 2020; 53 (2): 335-52 e8.
59, del Alamo D, Rouault H, and Schweisguth F. Mechanism and significance of cis-inhibition in Notch signalling. Curr Biol. 2011: 21 (1): R40-7.
60. Lee D, Pearsall R S, Das S, Dey S K, Godfrey V L, and Threadgill D W. Epiregulin is not essential for development of intestinal tumors but is required for protection from intestinal damage. Mol Cell Biol. 2004: 24 (20): 8907-16.
61. Park J Y, Su Y Q, Ariga M, Law E, Jin S L, and Conti M. EGF-like growth factors as mediators of LH action in the ovulatory follicle. Science. 2004: 303 (5658): 682-4.
62. Kim D, Peck A, Santer D, Patole P, Schwartz S M, Molitor J A, et al. Induction of interferon-alpha by scleroderma sera containing autoantibodies to topoisomerase I: association of higher interferon-alpha activity with lung fibrosis. Arthritis Rheum. 2008: 58 (7): 2163-73.
63. Kumar J P, and Moses K. The EGF receptor and notch signaling pathways control the initiation of the morphogenetic furrow during Drosophila eye development. Development. 2001: 128 (14): 2689-97.
64. Yoo A S, Bais C, and Greenwald I. Crosstalk between the EGFR and LIN-12/Notch pathways in C. elegans vulval development. Science. 2004: 303 (5658): 663-6.
65. Aguirre A, Rubio M E, and Gallo V. Notch and EGFR pathway interaction regulates neural stem cell number and self-renewal. Nature. 2010: 467 (7313): 323-7.
66. Wei K, Korsunsky I, Marshall J L, Gao A, Watts G F M, Major T, et al. Notch signalling drives synovial fibroblast identity and arthritis pathology. Nature. 2020: 582 (7811): 259-64.
67. Iwamoto N, Distler J H, and Distler O. Tyrosine kinase inhibitors in the treatment of systemic sclerosis: from animal models to clinical trials. Curr Rheumatol Rep. 2011: 13 (1): 21-7.
68. Gordon J K, Martyanov V, Magro C, Wildman H F, Wood T A, Huang W T, et al. Nilotinib (Tasigna) in the treatment of early diffuse systemic sclerosis: an open-label, pilot clinical trial. Arthritis Res Ther. 2015:17:213.
69, van den Hoogen F, Khanna D, Fransen J, Johnson S R, Baron M, Tyndall A, et al. 2013 classification criteria for systemic sclerosis: an American college of rheumatology/European league against rheumatism collaborative initiative. Ann Rheum Dis. 2013: 72 (11): 1747-55.
70. Butler A. Hoffman P. Smibert P. Papalexi E, and Satija R. Integrating single-cell transcriptomic data across different conditions, technologies, and species. Nature biotechnology. 2018.
71. Stuart T, Butler A, Hoffman P, Hafemeister C, Papalexi E, Mauck W M, 3rd, et al. Comprehensive Integration of Single-Cell Data. Cell. 2019: 177 (7): 1888-902 e21.
72. Hubner R H, Gitter W, El Mokhtari N E, Mathiak M, Both M, Bolte H, et al. Standardized quantification of pulmonary fibrosis in histological samples. Biotechniques. 2008: 44 (4): 507-11, 14-7.
73. Sefik E, Israelow B, Mirza H, Zhao J, Qu R, Kaffe E, et al. A humanized mouse model of chronic COVID-19. Nature biotechnology. 2021.
74. Rongvaux A, Willinger T, Martinek J, Strowig T, Gearty S V, Teichmann L L, et al. Development and function of human innate immune cells in a humanized mouse model. Nature biotechnology. 2014: 32 (4): 364-72.
75. Gerli M F M, Moyle L A, Benedetti S, Ferrari G, Ucuncu E, Ragazzi M, et al. Combined Notch and PDGF Signaling Enhances Migration and Expression of Stem Cell Markers while Inducing Perivascular Cell Features in Muscle Satellite Cells. Stem Cell Reports. 2019: 12 (3): 461-
76. Chua S L, See Too W C, Khoo B Y, and Few L L. UBC and YWHAZ as suitable reference genes for accurate normalisation of gene expression using MCF7, HCT116 and HepG2 cell lines. Cytotechnology. 2011: 63 (6): 645-54.
77. Livak K J, and Schmittgen T D. Analysis of relative gene expression data using real-time quantitative PCR and the 2 (−Delta Delta C(T)) Method. Methods. 2001: 25 (4): 402-8.
78. Bhattacharyya S, Tamaki Z, Wang W, Hinchcliff M, Hoover P, Getsios S, et al. FibronectinEDA promotes chronic cutaneous fibrosis through Toll-like receptor signaling. Sci Transl Med. 2014: 6 (232): 232ra50.
79. Sampaio-Pinto V, Rodrigues S C, Laundos T L, Silva E D, Vasques-Novoa F, Silva A C, et al. Neonatal Apex Resection Triggers Cardiomyocyte Proliferation, Neovascularization and Functional Recovery Despite Local Fibrosis. Stem Cell Reports. 2018: 10 (3): 860-74.
80. Garbers, C, et al. Inhibition of classic signaling is a novel function of soluble glycoprotein 130 (sgp130), which is controlled by the ratio of interleukin 6 and soluble interleukin 6 receptor. J Biol Chem 286, 42959-42970, doi: 10.1074/jbc.M111.295758 (2011).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

List of Sequences

SEQ ID NO: 1 hEreg NAb1 heavy chain variable region (VH)
HLQLQESGPGLVKSSETLSLTCSVSGGSISSSGYYWGWIRQPPGKGLEWIGSFYYSGN
TYYNPSLKSRVTISADTSKSQYSLKLSSVTAADTAVYYCARHPFNWNDHYHYMDVW
GNGTTVTVSS
SEQ ID NO: 2 hEreg NAb1 HCDR1
GGSISSSGYY
SEQ ID NO: 3 hEreg NAb1 HCDR2
FYYSGNT
SEQ ID NO: 4 hEreg NAb1 HCDR3
ARHPFNWNDHYHYMDV
SEQ ID NO: 5 Nucleotide sequence encoding hEreg NAb1 heavy chain
variable region (VH)
CACCTGCAACTGCAGGAGTCGGGCCCAGGACTGGTGAAGTCTTCGGAGACCCTG
TCCCTCACCTGCTCTGTCTCTGGTGGCTCCATCAGCAGTAGTGGTTACTACTGGG
GCTGGATCCGCCAGCCCCCAGGGAAGGGCCTGGAGTGGATTGGGAGTTTCTATT
ATAGTGGGAACACCTACTACAACCCGTCCCTCAAGAGTCGAGTCACCATATCCGC
AGACACGTCCAAGAGCCAGTACTCCCTGAAGCTGAGTTCTGTGACCGCCGCAGA
CACGGCTGTGTATTATTGTGCGAGACATCCGTTCAACTGGAACGACCACTATCAC
TACATGGACGTCTGGGGCAACGGGACCACGGTCACCGTCTCCTCA
SEQ ID NO: 6
hEreg NAb1 light chain variable region (VL)
DIQMTQSPSSLSASVGDSVTITCRASQSISNYLNWYQKKPGKAPKVLIYAASSLQSGV
PSRFSGSGSGTDFTLTISSLQPEDFATYYCQQSYITSITFGQGTRLEIK
SEQ ID NO: 7
hEreg NAb1 LCDR1
QSISNY
SEQ ID NO: 8
hEreg NAb1 LCDR2
AAS
SEQ ID NO: 9
hEreg NAb1 LCDR3
QQSYITSIT
SEQ ID NO: 10
Nucleotide sequence encoding hEreg NAb1 light chain
variable region (VL)
GACATCCAGATGACCCAGTCTCCATCCTCCCTGTCTGCATCTGTGGGAGACAGCG
TCACCATCACTTGCCGGGCAAGTCAGAGCATTAGCAACTATTTAAATTGGTATCA
GAAGAAACCAGGGAAAGCCCCTAAGGTCCTGATCTATGCTGCATCCAGTTTGCA
AAGTGGAGTCCCATCAAGGTTCAGTGGCAGTGGATCTGGAACAGATTTCACTCTC
ACCATCAGCAGTCTGCAACCTGAAGATTTTGCAACTTACTACTGTCAACAGAGTT
ACATAACCTCGATCACCTTCGGCCAAGGGACACGACTGGAGATTAAA
SEQ ID NO: 11 human UBC forward primer
CGTCACTTGACAATGCAG
SEQ ID NO: 12 human UBC reverse primer
TGTTTTCCAGCAAAGATCAG
SEQ ID NO: 13 human COL1A1 forward primer
GCTATGATGAGAAATCAACCG
SEQ ID NO: 14 human COL1A1 reverse primer
TCATCTCCATTCTTTCCAGG
SEQ ID NO: 15 human FN1EDA (78) forward primer
TAAAGGACTGGCATTCACTGA
SEQ ID NO: 16 human FN1EDA (78) reverse primer
GTGCAAGGCAACCACACTGAC
SEQ ID NO: 17 human TNC forward primer
GTGGGATCCTCTAGACATTG
SEQ ID NO: 18 human TNC reverse primer
GTGATCTCTCCCTCATCTTC
SEQ ID NO: 19 human EGF forward primer
CAGTTGATCCAGTAGAAAGG
SEQ ID NO: 20 human EGF reverse primer
TGTACTGAATCCAAAACAGC
SEQ ID NO: 21 human TGFA forward primer
AGAAACAGTGGTCTGAAGAG
SEQ ID NO: 22 human TGFA reverse primer
ATTACAGGCCAAGTAGGAAG
SEQ ID NO: 23 human BTC forward primer
CAGAAGTCCTGAAACTAATGG
SEQ ID NO: 24 human BTC reverse primer
CAATGTAGCCTTCATCACAG
SEQ ID NO: 25 human HBEGF forward primer
GCTTATATACCTATGACCACAC
SEQ ID NO: 26 human HBEGF reverse primer
GTACCTAAACATGAGAAGCC
SEQ ID NO: 27 human EREG forward primer
GTTCAGACAGAAGACAATCC
SEQ ID NO: 28 human EREG reverse primer
GACTCATGTCCACCAGATAG
SEQ ID NO: 29 human AREG forward primer
AAAGAAAGAAAAAGGGAGGC
SEQ ID NO: 30 human AREG reverse primer
CATTTGCATGTTACTGCTTC
SEQ ID NO: 31 human EPGN forward primer
GCAAGCTGACAACATAGAAG
SEQ ID NO: 32 human EPGN reverse primer
GCAGTAACTGTTATGATCTTCC
SEQ ID NO: 33 human DLL4 forward primer
GTTACACAGTGAAAAGCCAG
SEQ ID NO: 34 human DLL4 reverse primer
CTCTCCTCTGATATCAAACAC
SEQ ID NO: 35 human NOV forward primer
TAGAAGTCTCTGACTCAAGTG
SEQ ID NO: 36 human NOV reverse primer
CCTTTCTTATCTGTTGGCTG
SEQ ID NO: 37 human JAG1 forward primer
ACTACTACTATGGCTTTGGC
SEQ ID NO: 38 human JAG1 reverse primer
ATAGCTCTGTTACATTCGGG
SEQ ID NO: 39 human NOTCH1 forward primer
AAGATATGCAGAACAACAGG
SEQ ID NO: 40 human NOTCHI reverse primer
TCCATATGATCCGTGATGTC
SEQ ID NO: 41 human NOTCH2 forward primer
GATGAATGATGGTACTACACC
SEQ ID NO: 42 human NOTCH2 reverse primer
CAGATTTTCCATGGTCATCC
SEQ ID NO: 43 human NOTCH3 forward primer
CTACAATGGTGATAACTGTGAG
SEQ ID NO: 44 human NOTCH3 reverse primer
CAGTCATCCTCATTAATCTCG
human NOTCH4 forward primer
GTCCCCCAGGTTTCATAG
SEQ ID NO: 45 human NOTCH4 forward primer
GTCCCCCAGGTTTCATAG
SEQ ID NO: 46 human NOTCH4 reverse primer
ACTCATCCACATCTTCGG
SEQ ID NO: 47 human HES1 forward primer
GCCTATTATGGAGAAAAGACG
SEQ ID NO: 48 human HES1 reverse primer
CTATCTTTCTTCAGAGCATCC
SEQ ID NO: 49 human HES4 forward primer
AAAACCCTCATCCTGGAC
SEQ ID NO: 50 human HES4 reverse primer
GTGTCTCACGGTCATCTC
SEQ ID NO: 51 mouse Ubc forward primer
GAGACGATGCAGATCTTTG
SEQ ID NO: 52 mouse Ubc reverse primer
ATGTTGTAGTCTGACAGGG
SEQ ID NO: 53 mouse Col1a1 (79) forward primer
GCTCCTCTTAGGGGCCACT
SEQ ID NO: 54 mouse Col1a1 (79) reverse primer
CCACGTCTCACCATTGGGG
SEQ ID NO: 55 mouse Fn1EDA (78) forward primer
AGATGCAGCAGATCCGCAT
SEQ ID NO: 56 mouse Fn1EDA (78) reverse primer
GTTCTTGCCCATCAGCACC
SEQ ID NO: 57 mouse Tnc forward primer
CAAAGAGACCTTCATCACAG
SEQ ID NO: 58 mouse Tnc reverse primer
GGCTCTTTGGAACATCTATC
SEQ ID NO: 59 mouse Egf forward primer
GTACTCTTGGGTGTGAAAAC
SEQ ID NO: 60 mouse Egf reverse primer
CAAGTTCGTGACATTGTTTC
SEQ ID NO: 61 mouse Tgfa forward primer
CTCTGAGACAGTGGTCTG
SEQ ID NO: 62 mouse Tgfa reverse primer
GTCCCAAGATGCCTTTTC
SEQ ID NO: 63 mouse Btc forward primer
AGACCAATATTGCTTAACGG
SEQ ID NO: 64 mouse Btc reverse primer
AACAAGCAACACACAGTTAC
SEQ ID NO: 65 mouse Hbegf forward primer
GAGCTATAGGAACCTTCAGAG
SEQ ID NO: 66 mouse Hbegf reverse primer
ACTGGAAATGAATGAAGACG
SEQ ID NO: 67 mouse Ereg forward primer
AATTCTGCAGATGTGAAGTG
SEQ ID NO: 68 mouse Ereg reverse primer
TATTCTTTGCTCAAGGGTTG
SEQ ID NO: 69 mouse Areg forward primer
GACATGCAATTGTCATCAAG
SEQ ID NO: 70 mouse Areg reverse primer
GACAAAGATAGTGACAGCTAC
SEQ ID NO: 71 mouse Epgn forward primer
ATAGAAGAACCTGTAGCTCTG
SEQ ID NO: 72 mouse Epgn reverse primer
CCGTATAACCAGTAAAGCATC
SEQ ID NO: 73 oligo for qPCR
d(T)₂₀

1-71. (canceled)

72. An isolated antibody, or an antigen-binding fragment thereof, that specifically binds to epiregulin, comprising three heavy chain complementarity determining regions (CDRs) (HCDR1, HCDR2 and HCDR3) contained within a heavy chain variable region (VH) comprising the amino acid sequence of SEQ ID NO: 1, or a sequence having at least 80% identity thereto; and/or three light chain CDRs (LCDR1, LCDR2 and LCDR3) contained within a light chain variable region (VL) comprising the amino acid sequence of SEQ ID NO: 6, or a sequence having at least 80% identity thereto.

73. The isolated antibody or antigen-binding fragment of claim 72, wherein the isolated antibody or antigen-binding fragment has one or more characteristics selected from:
a) specifically binds to an epidermal growth factor (EGF)-like domain of epiregulin;
b) specifically binds to human epiregulin;
c) specifically binds to epiregulin with a K_{D} of less than about 1×10⁻⁹ M, or less than about 1×10⁻¹⁰ M, or about 3.8×10⁻¹¹ M;
d) neutralizes epiregulin,
e) inhibits epiregulin's interaction with an ErbB receptor;
f) inhibits epiregulin-induced proliferation of a fibroblast;
g) does not specifically bind to mouse epiregulin; and
h) does not specifically bind to one or more other human EGFR ligands.

74. The isolated antibody or antigen-binding fragment of claim 72, comprising an HCDR1 comprising the amino acid sequence of SEQ ID NO: 2, an HCDR2 comprising the amino acid sequence of SEQ ID NO: 3, and/or an HCDR3 comprising the amino acid sequence of SEQ ID NO: 4; and/or a LCDR1 comprising the amino acid sequence of SEQ ID NO: 7, a LCDR2 comprising the amino acid sequence of SEQ ID NO: 8, and/or a LCDR3 comprising the amino acid sequence of SEQ ID NO: 9.

75. The isolated antibody or antigen-binding fragment of claim 72, comprising a VH comprising the amino acid sequence of SEQ ID NO: 1, or a sequence having at least 80% identity thereto; and/or a VL comprising the amino acid sequence of SEQ ID NO: 6, or a sequence having at least 80% identity thereto.

76. The isolated antibody or antigen-binding fragment of claim 72, wherein the antibody or antigen-binding fragment is a human antibody, a monoclonal antibody, a humanized antibody, a single chain antibody, a Fab, a Fab′, a F(ab′)2, a Fv, or a scFv.

77. The isolated antibody or antigen-binding fragment of claim 72, wherein the antibody is a humanized antibody.

78. The isolated antibody or antigen-binding fragment of claim 72, wherein the antibody or antigen-binding fragment is of IgG1, IgG2, IgG3, or IgG4 isotype.

79. An isolated antibody or antigen-binding fragment thereof that competes for binding to epiregulin with the antibody or antigen-binding fragment of claim 72.

80. An isolated antibody or antigen-binding fragment thereof that binds to the same epitope as the antibody or antigen-binding fragment of claim 72.

81. An isolated polynucleotide encoding the isolated antibody or antigen-binding fragment of claim 72.

82. A vector comprising the polynucleotide of claim 81.

83. A host cell comprising the polynucleotide of claim 81.

84. A method of producing an isolated antibody or antigen-binding fragment that specifically binds to epiregulin, wherein said method comprises culturing the host cell of claim 83, and isolating said antibody or antigen-binding fragment.

85. A chimeric antigen receptor (CAR) comprising an extracellular domain comprising an antigen-binding moiety that specifically binds to epiregulin, wherein the antigen-binding moiety comprises the antibody or antigen-binding fragment of claim 72.

86. An immune cell comprising the CAR of claim 85 on its cell surface.

87. A pharmaceutical composition comprising the antibody or antigen-binding fragment of claim 72, and a pharmaceutically acceptable carrier or diluent.

88. A kit comprising (i) the isolated antibody or antigen-binding fragment of claim 72, and (ii) packaging for the same.

89. A method of inhibiting an activity of epiregulin in a cell, comprising contacting the cell with an effective amount of the antibody or antigen-binding fragment of claim 72.

90. A method of reversing or preventing one or more changes in a cell associated with fibrosis, comprising contacting the cell with an effective amount of the antibody or antigen-binding fragment of claim 72.

91. A method of treating or preventing a fibrotic disease or disorder in a subject in need thereof, comprising administering to the subject a therapeutically effective amount of the antibody or antigen-binding fragment of claim 72.


SEQUENCE LISTING (73 sequences)

SEQ ID NO: 1 - protein, 124 aa, synthetic construct
HLQLQESGPGLVKSSETLSLTCSVSGGSISSSGYYWGWIRQPPGKGLEWIGSFYYSGNTYYNPSLKSRVTISADTSKSQYSLKLSSVTAADTAVYYCARHPFNWNDHYHYMDVWGNGTTVTVSS

SEQ ID NO: 2 - protein, 10 aa, synthetic construct
GGSISSSGYY

SEQ ID NO: 3 - protein, 7 aa, synthetic construct
FYYSGNT

SEQ ID NO: 4 - protein, 16 aa, synthetic construct
ARHPFNWNDHYHYMDV

SEQ ID NO: 5 - DNA, 372 bp, synthetic construct
  source 1..372: mol_type = other DNA
cacctgcaactgcaggagtcgggcccaggactggtgaagtcttcggagaccctgtccctcacctgctctgtctctggtggctccatcagcagtagtggttactactggggctggatccgccagcccccagggaagggcctggagtggattgggagtttctattatagtgggaacacctactacaacccgtccctcaagagtcgagtcaccatatccgcagacacgtccaagagccagtactccctgaagctgagttctgtgaccgccgcagacacggctgtgtattattgtgcgagacatccgttcaactggaacgaccactatcactacatggacgtctggggcaacgggaccacggtcaccgtctcctca

SEQ ID NO: 6 - protein, 107 aa, synthetic construct
DIQMTQSPSSLSASVGDSVTITCRASQSISNYLNWYQKKPGKAPKVLIYAASSLQSGVPSRFSGSGSGTDFTLTISSLQPEDFATYYCQQSYITSITFGQGTRLEIK

SEQ ID NO: 7 - protein, 6 aa, synthetic construct
QSISNY

SEQ ID NO: 8 - skipped

SEQ ID NO: 9 - protein, 9 aa, synthetic construct
QQSYITSIT

SEQ ID NO: 10 - DNA, 321 bp, synthetic construct
  source 1..321: mol_type = other DNA
gacatccagatgacccagtctccatcctccctgtctgcatctgtgggagacagcgtcaccatcacttgccgggcaagtcagagcattagcaactatttaaattggtatcagaagaaaccagggaaagcccctaaggtcctgatctatgctgcatccagtttgcaaagtggagtcccatcaaggttcagtggcagtggatctggaacagatttcactctcaccatcagcagtctgcaacctgaagattttgcaacttactactgtcaacagagttacataacctcgatcaccttcggccaagggacacgactggagattaaa

SEQ ID NO: 11 - DNA, 18 bp, synthetic construct
  source 1..18: mol_type = other DNA
cgtcacttgacaatgcag

SEQ ID NO: 12 - DNA, 20 bp, synthetic construct
  source 1..20: mol_type = other DNA
tgttttccagcaaagatcag

SEQ ID NO: 13 - DNA, 21 bp, synthetic construct
  source 1..21: mol_type = other DNA
gctatgatgagaaatcaaccg

SEQ ID NO: 14 - DNA, 20 bp, synthetic construct
  source 1..20: mol_type = other DNA
tcatctccattctttccagg

SEQ ID NO: 15 - DNA, 21 bp, synthetic construct
  source 1..21: mol_type = other DNA
taaaggactggcattcactga

SEQ ID NO: 16 - DNA, 21 bp, synthetic construct
  source 1..21: mol_type = other DNA
gtgcaaggcaaccacactgac

SEQ ID NO: 17 - DNA, 20 bp, synthetic construct
  source 1..20: mol_type = other DNA
gtgggatcctctagacattg

SEQ ID NO: 18 - DNA, 20 bp, synthetic construct
  source 1..20: mol_type = other DNA
gtgatctctccctcatcttc

SEQ ID NO: 19 - DNA, 20 bp, synthetic construct
  source 1..20: mol_type = other DNA
cagttgatccagtagaaagg

SEQ ID NO: 20 - DNA, 20 bp, synthetic construct
  source 1..20: mol_type = other DNA
tgtactgaatccaaaacagc

SEQ ID NO: 21 - DNA, 20 bp, synthetic construct
  source 1..20: mol_type = other DNA
agaaacagtggtctgaagag

SEQ ID NO: 22 - DNA, 20 bp, synthetic construct
  source 1..20: mol_type = other DNA
attacaggccaagtaggaag

SEQ ID NO: 23 - DNA, 21 bp, synthetic construct
  source 1..21: mol_type = other DNA
cagaagtcctgaaactaatgg

SEQ ID NO: 24 - DNA, 20 bp, synthetic construct
  source 1..20: mol_type = other DNA
caatgtagccttcatcacag

SEQ ID NO: 25 - DNA, 22 bp, synthetic construct
  source 1..22: mol_type = other DNA
gcttatatacctatgaccacac

SEQ ID NO: 26 - DNA, 20 bp, synthetic construct
  source 1..20: mol_type = other DNA
gtacctaaacatgagaagcc

SEQ ID NO: 27 - DNA, 20 bp, synthetic construct
  source 1..20: mol_type = other DNA
gttcagacagaagacaatcc

SEQ ID NO: 28 - DNA, 20 bp, synthetic construct
  source 1..20: mol_type = other DNA
gactcatgtccaccagatag

SEQ ID NO: 29 - DNA, 20 bp, synthetic construct
  source 1..20: mol_type = other DNA
aaagaaagaaaaagggaggc

SEQ ID NO: 30 - DNA, 20 bp, synthetic construct
  source 1..20: mol_type = other DNA
catttgcatgttactgcttc

SEQ ID NO: 31 - DNA, 20 bp, synthetic construct
  source 1..20: mol_type = other DNA
gcaagctgacaacatagaag

SEQ ID NO: 32 - DNA, 22 bp, synthetic construct
  source 1..22: mol_type = other DNA
gcagtaactgttatgatcttcc

SEQ ID NO: 33 - DNA, 20 bp, synthetic construct
  source 1..20: mol_type = other DNA
gttacacagtgaaaagccag

SEQ ID NO: 34 - DNA, 21 bp, synthetic construct
  source 1..21: mol_type = other DNA
ctctcctctgatatcaaacac

SEQ ID NO: 35 - DNA, 21 bp, synthetic construct
  source 1..21: mol_type = other DNA
tagaagtctctgactcaagtg

SEQ ID NO: 36 - DNA, 20 bp, synthetic construct
  source 1..20: mol_type = other DNA
cctttcttatctgttggctg

SEQ ID NO: 37 - DNA, 20 bp, synthetic construct
  source 1..20: mol_type = other DNA
actactactatggctttggc

SEQ ID NO: 38 - DNA, 20 bp, synthetic construct
  source 1..20: mol_type = other DNA
atagctctgttacattcggg

SEQ ID NO: 39 - DNA, 20 bp, synthetic construct
  source 1..20: mol_type = other DNA
aagatatgcagaacaacagg

SEQ ID NO: 40 - DNA, 20 bp, synthetic construct
  source 1..20: mol_type = other DNA
tccatatgatccgtgatgtc

SEQ ID NO: 41 - DNA, 21 bp, synthetic construct
  source 1..21: mol_type = other DNA
gatgaatgatggtactacacc

SEQ ID NO: 42 - DNA, 20 bp, synthetic construct
  source 1..20: mol_type = other DNA
cagattttccatggtcatcc

SEQ ID NO: 43 - DNA, 22 bp, synthetic construct
  source 1..22: mol_type = other DNA
ctacaatggtgataactgtgag

SEQ ID NO: 44 - DNA, 21 bp, synthetic construct
  source 1..21: mol_type = other DNA
cagtcatcctcattaatctcg

SEQ ID NO: 45 - DNA, 18 bp, synthetic construct
  source 1..18: mol_type = other DNA
gtcccccaggtttcatag

SEQ ID NO: 46 - DNA, 18 bp, synthetic construct
  source 1..18: mol_type = other DNA
actcatccacatcttcgg

SEQ ID NO: 47 - DNA, 21 bp, synthetic construct
  source 1..21: mol_type = other DNA
gcctattatggagaaaagacg

SEQ ID NO: 48 - DNA, 21 bp, synthetic construct
  source 1..21: mol_type = other DNA
ctatctttcttcagagcatcc

SEQ ID NO: 49 - DNA, 18 bp, synthetic construct
  source 1..18: mol_type = other DNA
aaaaccctcatcctggac

SEQ ID NO: 50 - DNA, 18 bp, synthetic construct
  source 1..18: mol_type = other DNA
gtgtctcacggtcatctc

SEQ ID NO: 51 - DNA, 19 bp, synthetic construct
  source 1..19: mol_type = other DNA
gagacgatgcagatctttg

SEQ ID NO: 52 - DNA, 19 bp, synthetic construct
  source 1..19: mol_type = other DNA
atgttgtagtctgacaggg

SEQ ID NO: 53 - DNA, 19 bp, synthetic construct
  source 1..19: mol_type = other DNA
gctcctcttaggggccact

SEQ ID NO: 54 - DNA, 19 bp, synthetic construct
  source 1..19: mol_type = other DNA
ccacgtctcaccattgggg

SEQ ID NO: 55 - DNA, 19 bp, synthetic construct
  source 1..19: mol_type = other DNA
agatgcagcagatccgcat

SEQ ID NO: 56 - DNA, 19 bp, synthetic construct
  source 1..19: mol_type = other DNA
gttcttgcccatcagcacc

SEQ ID NO: 57 - DNA, 20 bp, synthetic construct
  source 1..20: mol_type = other DNA
caaagagaccttcatcacag

SEQ ID NO: 58 - DNA, 20 bp, synthetic construct
  source 1..20: mol_type = other DNA
ggctctttggaacatctatc

SEQ ID NO: 59 - DNA, 20 bp, synthetic construct
  source 1..20: mol_type = other DNA
gtactcttgggtgtgaaaac

SEQ ID NO: 60 - DNA, 20 bp, synthetic construct
  source 1..20: mol_type = other DNA
caagttcgtgacattgtttc

SEQ ID NO: 61 - DNA, 18 bp, synthetic construct
  source 1..18: mol_type = other DNA
ctctgagacagtggtctg

SEQ ID NO: 62 - DNA, 18 bp, synthetic construct
  source 1..18: mol_type = other DNA
gtcccaagatgccttttc

SEQ ID NO: 63 - DNA, 20 bp, synthetic construct
  source 1..20: mol_type = other DNA
agaccaatattgcttaacgg

SEQ ID NO: 64 - DNA, 20 bp, synthetic construct
  source 1..20: mol_type = other DNA
aacaagcaacacacagttac

SEQ ID NO: 65 - DNA, 21 bp, synthetic construct
  source 1..21: mol_type = other DNA
gagctataggaaccttcagag

SEQ ID NO: 66 - DNA, 20 bp, synthetic construct
  source 1..20: mol_type = other DNA
actggaaatgaatgaagacg

SEQ ID NO: 67 - DNA, 20 bp, synthetic construct
  source 1..20: mol_type = other DNA
aattctgcagatgtgaagtg

SEQ ID NO: 68 - DNA, 20 bp, synthetic construct
  source 1..20: mol_type = other DNA
tattctttgctcaagggttg

SEQ ID NO: 69 - DNA, 20 bp, synthetic construct
  source 1..20: mol_type = other DNA
gacatgcaattgtcatcaag

SEQ ID NO: 70 - DNA, 21 bp, synthetic construct
  source 1..21: mol_type = other DNA
gacaaagatagtgacagctac

SEQ ID NO: 71 - DNA, 21 bp, synthetic construct
  source 1..21: mol_type = other DNA
atagaagaacctgtagctctg

SEQ ID NO: 72 - DNA, 21 bp, synthetic construct
  source 1..21: mol_type = other DNA
ccgtataaccagtaaagcatc

SEQ ID NO: 73 - DNA, 20 bp, synthetic construct
  source 1..20: mol_type = other DNA
tttttttttttttttttttt